(19) United States
(12) Patent Application Publication
(10) Pub. No.: US 2023/0233584 A1
(43) Pub. Date: Jul. 27, 2023

(54) EXTENDED RELEASE 5-HT RECEPTOR AGONISTS FOR NEUROLOGICAL CONDITIONS

(71) Applicant: Diamond Therapeutics Inc., Toronto (CA)

(72) Inventors: Judith BLUMSTOCK, Toronto (CA); Guy Andrew HIGGINS, Toronto (CA); Edward Moncrieff SELLERS, Toronto (CA)

(21) Appl. No.: 18/102,268

(22) Filed: Jan. 27, 2023

Related U.S. Application Data
(63) Continuation of application No. PCT/IB2021/000488, filed on Jul. 28, 2021.
(60) Provisional application No. 63/058,386, filed on Jul. 29, 2020.

(51) Int. Cl. A61K 31/661; A61K 31/404; A61P 25/22; A61P 25/24

(52) U.S. Cl. CPC A61K 31/661; A61K 31/404; A61P 25/22; A61P 25/24

(57) ABSTRACT

Disclosed herein are compositions and methods of treating neuropsychiatric and cognitive conditions with single, repeat and/or extended release 5-HT receptor agonist dosage forms.

### CROSS-REFERENCE

This application is a bypass continuation of International Application No: PCT/IB2021/000488, filed on Jul. 28, 2021, which claims the benefit of U.S. Provisional Patent Application No. 63/058,386 filed on Jul. 29, 2020, which is incorporated herein by reference in its entirety.

### BACKGROUND

The serotonin (5-HT) receptors are a group of G protein-coupled receptors (GPCRs) and ligand-gated ion channels found in the central and peripheral nervous systems. Activation of 5-HT receptors can substantially influence brain function.

### SUMMARY

Provided in certain embodiments here are (e.g., pharmaceutical) compositions, such as comprising a 5HT receptor agonists. Provided herein in some embodiments is a method for treating or managing a mental, a behavioral, or a neuropsychiatric condition, or, the symptoms thereof, in an individual in need thereof, comprising administering to the individual a (e.g., therapeutically) effective amount of one or more 5-hydroxytryptamine (5-HT) receptor agonist, or a pharmaceutically acceptable salt, solvate, metabolite, derivative, or prodrug thereof (e.g., for an extended period of time). In some embodiments, the compositions and methods are for single, repeat, or extended use.

In certain embodiments, (e.g., pharmaceutical) compositions provided herein and/or methods provided herein (e.g., are configured to) provide a prolonged and/or repeated exposure in an individual to a 5HT receptor agonist. In some embodiments, the repeated exposure (e.g., a repeat dosing regimen) of the composition provided herein is provided or maintained at approximately the same dose and/or concentration of the 5HT receptor agonist (e.g., as an initial dose or subsequent dose of the 5HT receptor agonist). In some embodiments, the individual is administered a repeat dosing regimen. In some embodiments, the repeat dosing regimen comprises at least one ascending dose of the 5HT receptor agonist. In some embodiments, the repeat dosing regimen comprises at least one descending dose of the 5HT receptor agonist. In some embodiments, the repeat dosing regimen (e.g., regardless of the concentration (e.g. ascending, descending, or same concentration) comprises a dose of the 5-HT receptor agonist that is (i) at or above a minimum therapeutically effective threshold and (ii) below a hallucinogenic threshold of the active 5-HT receptor agonist in the individual.

In some embodiments, a 5HT receptor agonist is provided to an individual at a first time point and a second time point. In some embodiments, the first time and the second time are within a 24 hour period (e.g., within a 4 hour period, within a 6 hour period, within an 8 hour period, or the like). In some embodiments, the first time and the second time are on or after a 24 hour period (e.g., the first time is more than or equal to 24 hours after the second time, the first time is more than or equal to 48 hours after the second time, or the like). In certain embodiments, a composition provided herein comprises a first dosage form comprising a 5HT receptor agonist and a second dosage form comprising a 5HT receptor agonist (e.g., wherein the 5HT receptor agonists of the first and second dosage forms are the same or different). In some embodiments, a first dosage form is administered on a first day and a second dosage form is administered on a second day that is at least 1 day after the first day (e.g., at least 2 days after the first day, at least 3 days after the first day, at least 4 days after the first day, at least 5 days after the first day, at least 6 days after the first day, or at least at least 7 days after the first day). In some embodiments, additional dosage forms are also administered, such as on intervening days and/or subsequent to administration of the second dosage form.

In certain embodiments, a composition (or dosage form) provided herein is a controlled release or extended release composition (or dosage form). In some instances, the controlled release or extended release composition (or dosage form), or component thereof, provides exposure to a 5HT receptor agonist for an extended period of time, such as for at least 30 minutes, at least 1 hour, at least 2 hours, or longer. In some instances, the exposure is at or above a minimum effective (e.g., serum) concentration of an active 5HT receptor agonist). In certain instances, the exposure is below a maximum concentration, such as wherein the maximum concentration is a concentration that results in adverse events, such as, for example, hallucination, changes in perception, and/or changes in physical function, in an individual receiving the composition or dosage form.

In some instances, the composition (or dosage form) comprises an extended or controlled release component (e.g., comprising a 5HT receptor agonist, such as whereby a 5HT receptor agonist is released in a controlled or extended release manner when exposed to a biological sample, or an analog therein, such as a buffer, e.g., described herein) and an immediate release component (e.g., comprising a 5HT receptor agonist, such as whereby a 5HT receptor agonist is released in an immediate release manner when exposed to a biological sample, or an analog there, such as a buffer, e.g., described herein). In certain embodiments, a method provided herein comprises administering a first composition comprising a 5HT receptor agonist and a second composition comprising a 5HT receptor agonist, wherein the 5HT receptor agonist of the first composition is formulated for controlled or extended release and the 5HT receptor agonist second composition is formulated for immediate release. In some embodiments, the first and second compositions are configured together in a single dosage form, or are configured separately in discrete dosage forms. In some embodiments, the composition or method comprises a 5HT receptor agonist and a second agent, wherein the second agent is a 5HT receptor antagonist.

In some instances, repeated and/or prolonged delivery of a 5HT receptor agonist, such as via a composition provided herein, provides certain behavioral results, such as distinct from certain results seen when 5HT receptor agonists are administered in an immediate release formulation. For example, in some instances, immediate release of a 5HT receptor agonist provides for rapid results, such as rapid improvement in cognitive capability and/or motivation. In certain instances, repeated and prolonged delivery of 5HT receptor agonists results in an improved ability to respond to stressors. In certain instances, repeated and prolonged delivery of 5HT receptor agonists results in improved attention. In certain instances, repeated and prolonged delivery of 5HT receptor agonists results in improved cognition.

In some embodiments, the method comprises maintaining a level of an active 5-HT receptor agonist (i) at or above a minimum therapeutically effective threshold and (ii) below a hallucinogenic threshold of the active 5-HT receptor agonist in the individual for more than or equal to two hours. In some embodiments, the method comprises administering to the individual a dose of the 5-HT receptor agonist that is a single (e.g., full) dose (e.g., and causes an adverse event, such as, for example a hallucination). In some embodiments, the method comprises administering to the individual a dose of the 5-HT receptor agonist more frequently (e.g. as a half dose bi-daily, a third of a dose three times a day, or a fourth of a dose four times a day) or as an extended release, such as, for example, to avoid an adverse event (e.g., hallucinations) associated with the entire amount of a single (e.g., full) dose (e.g., taken at once). In some embodiments, the method comprises frequently administering to the individual one or more dose of the 5-HT receptor agonist (e.g., half the dose twice daily, a third of the dose three times daily, or a fourth of the dose four times daily) (e.g., thereby avoiding an adverse event, such as, a hallucinogen). In some embodiments, the method comprises administering an extended release composition (e.g., as described herein) (e.g., thereby avoiding an adverse event, such as, a hallucinogen).

In some embodiments, the method is for treating a mental, behavioral, or neuropsychiatric condition, or the symptoms thereof. In some embodiments, the method is for managing a mental, a behavioral, or a neuropsychiatric condition, or the symptoms thereof. In some embodiments, the method is for treating and managing a mental, behavioral, or neuropsychiatric condition, or the symptoms thereof. In some embodiments, the individual is suffering from or susceptible to the mental, a behavioral, or a neuropsychiatric condition. In certain instances, the symptoms of the mental, a behavioral, or a neuropsychiatric condition are physical, behavioral, emotional, mental, or a combination thereof.

In some embodiments, the mental, the behavioral, or the neuropsychiatric condition is any disease or disorder provided herein. In some embodiments, the mental, the behavioral, or the neuropsychiatric condition is a symptom (e.g., provided herein) of any disease or disorder provided herein.

In some embodiments, the mental, the behavioral, or the neuropsychiatric condition is a chronic condition.

In some embodiments, the mental, the behavioral, or the neuropsychiatric condition is a Diagnostic and Statistical Manual of Mental Disorders (DSM-5) category disease or disorder). In some embodiments, the mental, the behavioral, or the neuropsychiatric condition is a non-DSM-5 category disease or disorder.

In some embodiments, the mental, the behavioral, or the neuropsychiatric condition is an attention (e.g., an attention deficit) or a cognitive (e.g., neurocognitive) disorder or condition.

In some embodiments, the mental, the behavioral, or the neuropsychiatric condition is a neurocognitive disorder or condition.

In some embodiments, the mental, the behavioral, or the neuropsychiatric condition is an attention deficit disorder or condition.

In some embodiments, (e.g., provided herein is a method (e.g., as described herein)) for treating a mental, behavioral, or neuropsychiatric condition comprising administering a (e.g., therapeutically effective amount of a 5-HT receptor agonist (e.g., psilocybin)) to an individual (e.g., in need thereof) and the mental, the behavioral, or the neuropsychiatric condition is a stress-related, stress-induced, or stressor-related disorder or condition (e.g., post-traumatic stress disorder (PTSD), prolonged grief disorder (PGD), social anxiety, clinical depression, post-surgical depression, or depression from chronic pain). In some embodiments, the mental, the behavioral, or the neuropsychiatric condition is a stress-related, stress-induced, or stressor-related disorder or condition (e.g. as evidenced by Chronic Social Defeat in animals).

In some embodiments, (e.g., provided herein is a method for treating a mental, behavioral, or neuropsychiatric condition comprising administering a (e.g., therapeutically effective amount of a 5-HT receptor agonist (e.g., psilocybin)) to an individual (e.g., in need thereof) and the mental, the behavioral, or the neuropsychiatric condition is selected from the group consisting of addiction (e.g., a weight management disorder), anxiety, apathy, attention (e.g., the lack thereof), and depression (e.g., moderate depression, prolonged grief disorder (PGD), social anxiety, post-surgical depression, or depression from chronic pain).

In some embodiments, the mental, the behavioral, or the neuropsychiatric condition is PTSD, constructive impulsivity, a phobia, a fear, or the like.

In some embodiments, the mental, the behavioral, or the neuropsychiatric condition is depression or a depression disorder.

In some embodiments, the mental, the behavioral, or the neuropsychiatric condition is major depressive disorder.

In some embodiments, the mental, the behavioral, or the neuropsychiatric condition is prolonged grief disorder (PGD), social anxiety, post-surgical depression, depression from chronic pain.

In some embodiments, the mental, the behavioral, or the neuropsychiatric condition is prolonged grief disorder (PGD).

In some embodiments, the mental, the behavioral, or the neuropsychiatric condition is social anxiety.

In some embodiments, the mental, the behavioral, or the neuropsychiatric condition is post-surgical depression.

In some embodiments, the mental, the behavioral, or the neuropsychiatric condition is depression from chronic pain.

In some embodiments, the mental, the behavioral, or the neuropsychiatric condition is a weight management disorder. In some embodiments, the individual maintains a desired body weight, controls weight gain, has increased weight loss, has diminished food addition, has reduced food cravings, has curbed impulsive eating, has increased metabolism, and/or decreases their caloric intake (e.g., through lower calorie foods).

In some embodiments, the method comprises maintaining the level of 5-HT receptor agonist in the individual for a period of time sufficient to treat or manage the mental, the behavioral, or the neuropsychiatric condition(s) of the individual. In some embodiments, the method comprises maintaining the level of 5-HT receptor agonist in the individual for a period of time sufficient to treat or manage the symptoms of the mental, the behavioral, or the neuropsychiatric condition(s) of the individual. In some embodiments, the method comprises maintaining the level of 5-HT receptor agonist in the individual for a period of more than or equal to two hours, more than or equal to 12 hours, more than or equal to 1 day, more than or equal to 7 days, or more than or equal to 14 days. In some embodiments, the method comprises maintaining the level of 5-HT receptor agonist in the individual for a period of more than or equal to 1 day.

Provided herein in certain instances is a method for increasing motivation in an individual in need thereof, comprising administering to the individual a therapeutically effective amount of one or more 5-hydroxytryptamine (5-HT) receptor agonist, or a pharmaceutically acceptable salt, solvate, metabolite, derivative, or prodrug thereof (e.g., for an extended period of time). In some embodiments, the method comprises maintaining a level of an active 5-HT receptor agonist (i) at or above a minimum therapeutically effective threshold and (ii) below a hallucinogenic threshold of the active 5-HT receptor agonist in the individual for more than or equal to two hours.

In some embodiments, the hallucinogenic threshold is a threshold of the 5-HT receptor agonist that produces an adverse event in the individual. In some embodiments, the adverse event is a clinically important effect in the individual. In some embodiments the adverse event is altered perception, altered cognition, impaired attention, drowsiness, confusion, or the like. In some embodiments, the altered perception is a visual alteration of perception, an auditory alteration of perception, a bodily alteration of perception, a temporal alteration of perception, or a spatial alteration of perception.

In some embodiments, the level of the active 5-HT receptor agonist in the individual is measured by obtaining a measurement of a physiological response or reaction (e.g., electroencephalogram (EEG), electrocardiogram (ECG or EKG), pulse rate, oximetry, or the like) of the individual (e.g., prior or subsequent to the administration of the one or more 5-HT receptor agonist).

In some embodiments, the level of active 5-HT receptor agonist in the individual is measured by obtaining a biological sample from the individual. In some embodiments, the biological sample is serum, plasma, whole blood, urine, or the like.

In some embodiments, the one or more 5-HT receptor agonist, or a pharmaceutically acceptable salt, solvate, metabolite, derivative, or prodrug thereof is a 5HT_{2A} and/or 5HT_{2C} receptor agonist. In some embodiments, the one or more 5-HT receptor agonist, or a pharmaceutically acceptable salt, solvate, metabolite, derivative, or prodrug thereof is a 5HT_{2A} receptor agonist. In some embodiments, the one or more 5-HT receptor agonist, or a pharmaceutically acceptable salt, solvate, metabolite, derivative, or prodrug thereof is a 5HT_{2C} receptor agonist. In some embodiments, the one or more 5-HT receptor agonist, or a pharmaceutically acceptable salt, solvate, metabolite, derivative, or prodrug thereof is a 5HT_{2A} and 5HT_{2C} receptor agonist.

In some embodiments, the one or more 5-HT receptor agonist, or a pharmaceutically acceptable salt, solvate, metabolite, derivative, or prodrug thereof is a hallucinogenic compound.

In some embodiments, the hallucinogenic compound is selected from the group consisting of lysergic acid diethylamide (LSD), dimethyltryptamine (DMT), psilocybin, and psilocin, or a pharmaceutically acceptable salt, solvate, metabolite, derivative, or prodrug thereof.

In some embodiments, the one or more 5-HT receptor agonist, or a pharmaceutically acceptable salt, solvate, metabolite, derivative, or prodrug thereof is selected from the group consisting of LSD, dimethyltryptamine (DMT), psilocybin, and psilocin, or a pharmaceutically acceptable salt, solvate, metabolite, derivative, or prodrug thereof.

In some embodiments, the one or more 5-HT receptor agonist is psilocybin or psilocin, or a pharmaceutically acceptable salt, solvate, metabolite, derivative, or prodrug thereof. In some embodiments, the active 5-HT receptor agonist is psilocin.

In some embodiments, the hallucinogenic compound produces a hallucinogenic effect in the individual. In some embodiments, the hallucinogenic compound, or a pharmaceutically acceptable salt, solvate, metabolite, derivative, or prodrug thereof, produces an adverse event or a clinically important effect in the individual in need thereof at or above the hallucinogenic threshold of the hallucinogenic compound. In some embodiments, the clinically important effect is a clinically important impairment of the individual, altered perception, altered cognition, impaired attention, drowsiness, and/or confusion. In some embodiments, the altered perception in the individual is a visual perception alteration, an auditory perception alteration, bodily perception alteration, a temporal perception alteration, or a spatial perception alteration. In some embodiments, the hallucinogenic threshold of the hallucinogenic compound is at or above a Cₘₐₓ above the hallucinogenic effective threshold of the active form of the hallucinogenic compound.

In some embodiments, the method comprises maintaining the level of 5-HT receptor agonist in the individual for a period of time sufficient to increase motivation in the individual. In some embodiments, the method comprises maintaining the level of 5-HT receptor agonist in the individual for a period of time sufficient to maintain motivation in the individual. In some embodiments, the method comprises maintaining the level of 5-HT receptor agonist in the individual for a period of more than or equal to two hours, more than or equal to 12 hours, more than or equal to 1 day, more than or equal to 7 days, or more than or equal to 14 days. In some embodiments, the method comprises maintaining the level of 5-HT receptor agonist in the individual for a period of more than or equal to 1 day.

In some instances, the method comprises administering to the individual the therapeutically effective amount of one or more 5-HT receptor agonist, or a pharmaceutically acceptable salt, solvate, metabolite, derivative, or prodrug thereof, at a dose such that the concentration of the active 5-HT receptor agonist remains below the hallucinogenic effective threshold (e.g., below the maximum plasma concentration (Cₘₐₓ)) of the active 5-HT receptor agonist in the individual. In some instances, the method comprises administering to the individual the therapeutically effective amount of one or more 5-HT receptor agonist, or a pharmaceutically acceptable salt, solvate, metabolite, derivative, or prodrug thereof, at dose such that the concentration of the active 5-HT receptor agonist remains above the therapeutically effective threshold (e.g., above the minimum plasma concentration (Cₘᵢₙ)) of the active 5-HT receptor agonist in the individual. In some instances, the method comprises administering to the individual the therapeutically effective amount of one or more 5-HT receptor agonist, or a pharmaceutically acceptable salt, solvate, metabolite, derivative, or prodrug thereof, at a dose sufficient to provide a Cₘₐₓ of the active 5-HT receptor agonist below the hallucinogenic effective threshold of the active 5-HT receptor agonist and a Cₘᵢₙ of the active 5-HT receptor agonist at least the therapeutically effective threshold of the active 5-HT receptor agonist in the individual.

In some embodiments, the level of the active 5-HT receptor agonist is maintained in the individual above the therapeutically effective threshold and below the hallucinogenic effective threshold of the active 5-HT receptor agonist. In some embodiments, the level of the active 5-HT receptor agonist in the individual is above the therapeutically effective threshold of the active 5-HT receptor agonist. In some embodiments, the level of the active 5-HT receptor agonist in the individual is below the hallucinogenic effective threshold of the active 5-HT receptor agonist. In some embodiments, the level of the active 5-HT receptor agonist in the individual is above the therapeutically effective threshold and below the hallucinogenic effective threshold of the active 5-HT receptor agonist.

In some embodiments, the level of active 5-HT receptor agonist is maintained in the individual at a concentration more than or equal to 0.01 ng/mL (e.g., more than 0.01 ng/mL, more than 0.05 ng/mL, more than 0.1 ng/mL, more than 0.5 ng/mL, more than 1 ng/mL, more than 5 ng/mL, or more than 10 ng/mL). In some embodiments, the level of active 5-HT receptor agonist is maintained in the individual at a concentration less than or equal to 10 ng/mL (e.g., less than 10 ng/mL, less than 5 ng/mL, less than 1 ng/mL, less than 0.5 ng/mL, less than 0.1 ng/mL, less than 0.05 ng/mL, or less than 0.01 ng/mL). In some embodiments, the level of the active 5-HT receptor agonist is maintained in the individual from about 0.01 ng/mL to about 10 ng/mL. In some embodiments, the level of the active 5-HT receptor agonist in the individual is from about 0.01 ng/mL to about 10 ng/mL.

In some embodiments, the level (e.g., Cₘₐₓ) of the active 5-HT receptor agonist (e.g., psilocin) in the individual is (maintained) at a level of at least about 0.001 ng/mL or more (e.g., 0.01 ng/mL or more, 0.1 ng/mL or more, 1 ng/mL or more, 10 ng/mL or more, 20 ng/mL or more, or 50 ng/mL or more). In some embodiments, the level (e.g., Cₘₐₓ) of the active 5-HT receptor agonist (e.g., psilocin) in the individual is (maintained) at a level of at least about 100 ng/mL or less (e.g., 50 ng/mL or less, 25 ng/mL or less, 15 ng/mL or less, 5 ng/mL or less, or 0.5 ng/mL or less). In some embodiments, the level (e.g., Cₘₐₓ) of the active 5-HT receptor agonist (e.g., psilocin) in the individual is (maintained) at a level of about 0.001 ng/mL to about 100 ng/mL. In some embodiments, the level (e.g., Cₘₐₓ) of the active 5-HT receptor agonist (e.g., psilocin) in the individual is (maintained) at a level of about 0.1 ng/mL to about 50 ng/mL. In some embodiments, the level (e.g., Cₘₐₓ) of the active 5-HT receptor agonist (e.g., psilocin) in the individual is (maintained) at a level of about 1 ng/mL to about 25 ng/mL. In some embodiments, the level (e.g., Cₘₐₓ) of the active 5-HT receptor agonist (e.g., psilocin) in the individual is (maintained) at a level of about 2 ng/mL to about 12 ng/mL. In some embodiments, the level (e.g., Cₘₐₓ) of the active 5-HT receptor agonist (e.g., psilocin) in the individual is (maintained) at a level of about 5 ng/mL to about 24 ng/mL. In some embodiments, the level (e.g., Cₘₐₓ) is measured after a dose of psilocybin (e.g., a dose provided herein) is administered to the individual.

In some embodiments, the level (e.g., Cₘₐₓ) of the active 5-HT receptor agonist (e.g., psilocin) is a sub-hallucinogenic level of the active 5-HT receptor agonist (e.g., psilocin).

In some embodiments, the level (e.g., Cₘₐₓ) of the active 5-HT receptor agonist (e.g., psilocin) is measured after a dose of at least 1 mg or more (e.g., 5 mg or more, 10 mg or more, 15 mg or more, or 20 mg or more) of the 5-HT receptor agonist (e.g., psilocybin) is administered to the individual.

In some embodiments, the dose of the 5-HT receptor agonist (e.g., psilocybin) is a sub-hallucinogenic dose of the 5-HT receptor agonist (e.g., psilocybin) or the active 5-HT receptor agonist (e.g., psilocin)

In some embodiments, psilocybin is administered orally. In some embodiments, is administered intravenously.

In some instances, a dose, pharmacokinetic parameter, or the like may change, such as, when using different formulations (e.g., as provided herein). In some instances, a dose, pharmacokinetic parameter, or the like may change, such as, when using different formulations (e.g., as provided herein), but the dose, pharmacokinetic parameter, or the like is generically sub-hallucinogenic. In some instances, a dose, pharmacokinetic parameter, or the like may change, such as, when using different formulations (e.g., as provided herein), but the dose, pharmacokinetic parameter, or the like is generically therapeutically effective and sub-hallucinogenic.

In some embodiments, the therapeutically effective amount of the one or more 5-HT receptor agonist, or a pharmaceutically acceptable salt, solvate, metabolite, derivative, or prodrug thereof, is administered to the individual in need thereof in an amount and/or formulation insufficient to provide a Cₘₐₓ of the active 5-HT receptor agonist of 10 ng/mL or more in the individual. In some embodiments, the therapeutically effective amount of the one or more 5-HT receptor agonist, or a pharmaceutically acceptable salt, solvate, metabolite, derivative, or prodrug thereof, is administered to the individual in need thereof in an amount and/or formulation insufficient to provide a Cₘₐₓ of the active 5-HT receptor agonist of 6 ng/mL or more in the individual. In some instances, the therapeutically effective amount of the one or more 5-HT receptor agonist, or a pharmaceutically acceptable salt, solvate, metabolite, derivative, or prodrug thereof, is administered to the individual in need thereof in an amount and/or formulation to provide a Cₘₐₓ of the active 5-HT receptor agonist of about 0.1 ng/mL to about 6 ng/mL in the individual. In some instances, the therapeutically effective amount of the one or more 5-HT receptor agonist, or a pharmaceutically acceptable salt, solvate, metabolite, derivative, or prodrug thereof, is administered to the individual in need thereof in an amount and/or formulation to provide a Cₘₐₓ of the active 5-HT receptor agonist of about 0.1 ng/mL to about 6 ng/mL, about 0.5 ng/mL to about 6 ng/mL, about 1 ng/mL to about 5.5 ng/mL, about 2 ng/mL to about 5 ng/mL, or the like in the individual.

In some embodiments, the therapeutically effective amount of the one or more 5-HT receptor agonist, or a pharmaceutically acceptable salt, solvate, metabolite, derivative, or prodrug thereof, is administered to the individual in need thereof in an amount and/or formulation to provide a plasma concentration of the active 5-HT receptor agonist of at least 0.1 ng/mL in the individual for at least 2 hours. In some embodiments, the therapeutically effective amount of the one or more 5-HT receptor agonist, or a pharmaceutically acceptable salt, solvate, metabolite, derivative, or prodrug thereof, is administered to the individual in need thereof in an amount and/or formulation to provide a plasma concentration of the active 5-HT receptor agonist of at least 0.1 ng/mL in the individual for at least 3 hours. In some embodiments, the therapeutically effective amount of the one or more 5-HT receptor agonist, or a pharmaceutically acceptable salt, solvate, metabolite, derivative, or prodrug thereof, is administered to the individual in need thereof in an amount and/or formulation to provide a plasma concentration of the active 5-HT receptor agonist that never exceeds 10 ng/mL for any period of time in the individual. In some embodiments, the therapeutically effective amount of the one or more 5-HT receptor agonist, or a pharmaceutically acceptable salt, solvate, metabolite, derivative, or prodrug thereof, is administered to the individual in need thereof in an amount and/or formulation to provide a plasma concentration of the active 5-HT receptor agonist of at most 10 ng/mL in the individual for at least 2 hours. In some embodiments, the therapeutically effective amount of the one or more 5-HT receptor agonist, or a pharmaceutically acceptable salt, solvate, metabolite, derivative, or prodrug thereof, is administered to the individual in need thereof in an amount and/or formulation to provide a plasma concentration of the active 5-HT receptor agonist of at least 0.1 ng/mL and at most 10 ng/mL in the individual for at least 3 hours at least 12 hours, at least 24 hours, at least 36 hours, at least 48 hours, at least 72 hours, at least 96 hours, at least 120 hours, at least 144 hours, or the like.

In some embodiments, the therapeutically effective amount of the one or more 5-HT receptor agonist, or a pharmaceutically acceptable salt, solvate, metabolite, derivative, or prodrug thereof, is administered to the individual in need thereof over an extended period of time. In some embodiments, the therapeutically effective amount of the one or more 5-HT receptor agonist, or a pharmaceutically acceptable salt, solvate, metabolite, derivative, or prodrug thereof, is administered to the individual over an extended period of time such that the level of active 5-HT receptor agonist does not exceed 10 ng/mL in the individual (e.g., for the entirety of the extended period of time). In some embodiments, the therapeutically effective amount of the one or more 5-HT receptor agonist, or a pharmaceutically acceptable salt, solvate, metabolite, derivative, or prodrug thereof, is administered to the individual in need thereof over an extended period of time such that the level of active 5-HT receptor agonist does not fall below 0.01 ng/mL in the individual (e.g., for the entirety of the extended period of time). In some embodiments, the therapeutically effective amount of the one or more 5-HT receptor agonist, or a pharmaceutically acceptable salt, solvate, metabolite, derivative, or prodrug thereof, is administered to the individual in need thereof over an extended period of time such that the level of active 5-HT receptor agonist does not exceed 10 ng/mL and does not fall below 0.01 ng/mL in the individual (e.g., for the entirety of the extended period of time). In some embodiments, the therapeutically effective amount of the one or more 5-HT receptor agonist, or a pharmaceutically acceptable salt, solvate, metabolite, derivative, or prodrug thereof, is administered to the individual in need thereof over an extended period of time, for example, such that the level of active 5-HT receptor agonist is (e.g., maintained at a level of) at least 0.01 ng/mL in the individual (e.g., for the entirety of the extended period of time). In some embodiments, the therapeutically effective amount of the one or more 5-HT receptor agonist, or a pharmaceutically acceptable salt, solvate, metabolite, derivative, or prodrug thereof, is administered to the individual in need thereof over an extended period of time, for example, such that the level of active 5-HT receptor agonist is (e.g., maintained at a level of) at least 0.01 ng/mL in the individual (e.g., for the entirety of the extended period of time) but not higher than a concentration (e.g., Cₘₐₓ) of the 5-HT receptor agonist that produces an adverse event (e.g., a hallucinogenic threshold) in the individual in need thereof.

In some embodiments, the extended period of time is for an entire treatment plan tailored for the individual in need thereof. In some embodiments, the extended period of time is one day, one week, two weeks, one month, six months, one year, or more. In some embodiments, the individual in need thereof is administered the one or more 5-HT receptor agonist, or the pharmaceutically acceptable salt, solvate, metabolite, derivative, or prodrug thereof, for at least one week. In some embodiments, the individual in need thereof is administered the one or more 5-HT receptor agonist, or the pharmaceutically acceptable salt, solvate, metabolite, derivative, or prodrug thereof, daily for a week, every other day for a week, two times a week, once a week, bi-weekly for a month, or the like.

In some embodiments, the therapeutically effective amount of the one or more 5-HT receptor agonist, or a pharmaceutically acceptable salt, solvate, metabolite, derivative, or prodrug thereof, is administered to the individual in need thereof as a controlled release formulation. In some embodiments, the therapeutically effective amount of the one or more 5-HT receptor agonist, or a pharmaceutically acceptable salt, solvate, metabolite, derivative, or prodrug thereof, is administered to the individual in need thereof as an extended release formulation. In some embodiments, the extended release formulation releases the active 5-HT receptor agonist in the individual. In some embodiments, the extended release formulation releases the active 5-HT receptor agonist in the individual such that the active 5-HT receptor agonist reaches a Cₘₐₓ below the hallucinogenic effective threshold in the individual. In some embodiments, the extended release formulation releases the active 5-HT receptor agonist in the individual such that the active 5-HT receptor agonist reaches a Cₘᵢₙ of at least the therapeutically effective threshold in the individual. In some embodiments, the extended release formulation releases the active 5-HT receptor agonist in the individual such that the active 5-HT receptor agonist reaches a Cₘₐₓ below the hallucinogenic effective threshold and a Cₘᵢₙ of at least the therapeutically effective threshold in the individual.

In some instances, the controlled release formulation comprises an extended release component (e.g., that releases the active 5-HT receptor agonist in the individual (e.g., reaching a Cₘₐₓ below the hallucinogenic effective threshold and a Cₘᵢₙ of at least the therapeutically effective threshold in the individual)). In some instances, the controlled release formulation comprises an immediate release component (e.g., that releases the active 5-HT receptor agonist in the individual (e.g., reaching a Cₘₐₓ below the hallucinogenic effective threshold and a Cₘᵢₙ, of at least the therapeutically effective threshold in the individual)). In some embodiments, the controlled release formulation comprises an extended release component (e.g., that releases the active 5-HT receptor agonist in the individual (e.g., reaching a Cₘₐₓ below the hallucinogenic effective threshold and a Cₘᵢₙ of at least the therapeutically effective threshold in the individual)) and an immediate release component (e.g., that releases the active 5-HT receptor agonist in the individual (e.g., reaching a Cₘₐₓ below the hallucinogenic effective threshold and a Cₘᵢₙ of at least the therapeutically effective threshold in the individual)).

In some embodiments, the immediate release component provides a dose of the active 5-HT receptor agonist such that the level of the active 5-HT receptor agonist in the individual is below the hallucinogenic effective threshold and above the therapeutically effective threshold in the individual. In some embodiments, the immediate release component provides an initial dose of the active 5-HT receptor agonist in the individual (e.g., below the hallucinogenic effective threshold and above the therapeutically effective threshold) that is maintained by the extended release component in the individual. In some embodiments, the immediate release component produces at least one effect in the individual. In some embodiments, an effect of the at least one effect is maintained in the individual by the extended release component.

In some embodiments, provided herein is a method for treating apathy or low motivation (e.g., or a condition (e.g., or the symptoms thereof) associated with apathy or low motivation, such as, for example, depression, anxiety, or the like) by administering an controlled release formulation of a 5-HT receptor agonist to an individual in need thereof. In some embodiments, treating apathy or low motivation in an individual in need thereof is improved when the individual is administered a composition or pharmaceutical formulation comprising an immediate release component and an extended release component.

In some embodiments, the immediate release component is an immediate-release coating. In some embodiments, the immediate-release coating provides immediate release of the active 5-HT receptor agonist. In some embodiments, the immediate-release coating surrounds the controlled release component. In some embodiments, the immediate-release coating and the controlled release component each release the active 5-HT receptor agonist.

In some instances, the controlled release formulation is any formulation provided herein. In some instances, the controlled release formulation is an oral formulation, a dermal formulation, a buccal formulation, a nasal formulation, an intraparietal (IP) formulation, or an inhalation formulation. In some embodiments, the oral formulation is in a solid form or a liquid form.

In some embodiments, the controlled release formulation releases the active 5-HT receptor agonist in the individual in need thereof for a period of at least two hours. In some embodiments, the controlled release formulation releases the active 5-HT receptor agonist in the individual in need thereof for a period of at most two hours. In some embodiments, the controlled release formulation releases the active 5-HT receptor agonist in the individual in need thereof for a period of at least one day. In some embodiments, the controlled release formulation releases the active 5-HT receptor agonist in the individual in need thereof for a period of at most one day. In some embodiments, the controlled release formulation releases the active 5-HT receptor agonist in the individual in need thereof for a period of two hours to one week.

In some embodiments, the extended release component releases the active 5-HT receptor agonist in the individual in need thereof for a period of at least two hours. In some embodiments, the extended release component releases the active 5-HT receptor agonist in the individual in need thereof for a period of at most two hours. In some embodiments, the extended release component releases the active 5-HT receptor agonist in the individual in need thereof for a period of at least one day. In some embodiments, the extended release component releases the active 5-HT receptor agonist in the individual in need thereof for a period of at most one day. In some embodiments, the extended release component releases the active 5-HT receptor agonist in the individual in need thereof for a period of two hours to one week.

In more specific embodiments, the method, (e.g., pharmaceutical) composition, formulation, or dosage form comprises a pharmaceutically acceptable excipient. In some embodiments, the method, (e.g., pharmaceutical) composition, formulation, or dosage form comprises one or more agents selected from the group consisting of surfactants, preservatives, flavoring agents, sweetening agents, and antifoaming agents.

In various embodiments provided herein, the (e.g., pharmaceutical) composition, formulation, or dosage of the (e.g., one or more) 5-HT receptor agonist is any suitable 5-HT receptor agonist, such as a 5-HT2 receptor agonist, including free bases thereof, salts thereof, prodrugs thereof, metabolites thereof, or the like. In specific embodiments, the one or more 5-HT receptor agonist is psilocybin or psilocin, or a pharmaceutically acceptable salt, solvate, metabolite, derivative, or prodrug thereof.

In certain embodiments, the (e.g., pharmaceutical) composition, formulation, or dosage form is a pharmaceutical composition. In specific embodiments the pharmaceutical composition is a dosage form, such as a discrete dosage form. In more specific embodiments, the pharmaceutical composition is a discrete oral dosage form.

In some embodiments, the therapeutically effective amount of 5-HT receptor agonist is an amount insufficient to provide a hallucinogenic experience (or other adverse effect) (e.g., in an average adult).

In some embodiments, the pharmaceutical composition is a low-dose pharmaceutical composition. In specific embodiments, the low-dose pharmaceutical composition is a pharmaceutical composition (e.g., dosage form) such that following administration to an individual in need thereof, the low-dose pharmaceutical composition provides a maximum plasma concentration (Cₘₐₓ) of the 5-HT receptor agonist (e.g., or active metabolite(s) thereof) of less than 6 ng/mL in the individual in need thereof. In further embodiments, the low-dose pharmaceutical composition is a pharmaceutical composition (e.g., dosage form) such that following administration to an individual in need thereof, the low-dose pharmaceutical composition provides a plasma concentration (Cₘₐₓ) of the 5-HT receptor agonist (e.g., or active metabolite(s) thereof) of at least a therapeutic level (e.g. 0.01 ng/mL), but not higher than a concentration (e.g., Cₘₐₓ) of the 5-HT receptor agonist that produces an adverse event (e.g., a hallucinogenic threshold) in the individual in need thereof. In more specific embodiments, following administration to an individual in need thereof, the low-dose pharmaceutical composition provides a maximum plasma concentration (Cₘₐₓ) of the 5-HT receptor agonist (e.g., or active metabolite(s) thereof) of at least 0.5 ng/mL and less than 6 ng/mL in the individual in need thereof (e.g., about 1 ng/mL to about 5.5 ng/mL, about 2 ng/mL to about 5 ng/mL, or the like).

In various embodiments, the pharmaceutical composition is formulated in any suitable manner. For example, in some embodiments, the pharmaceutical composition comprises a controlled release component (e.g., extended release component and/or an immediate release component). In specific embodiments, following administration to an individual in need thereof, the pharmaceutical composition provides a minimum plasma concentration (Cₘᵢₙ) of the 5-HT receptor agonist (e.g., or active metabolite(s) thereof) of about 0.1 ng/mL or more in the individual, wherein the minimum plasma concentration (Cₘᵢₙ) is determined at a time between 2 hours and 12 hours (or between 2 hours and 24 hours, or between 2 hours and 48 hours, or between 2 hours and 72 hours, or the like) after administration to the individual. In some embodiments, following administration to an individual in need thereof, the pharmaceutical composition provides a minimum plasma concentration (Cₘᵢₙ) of the 5-HT receptor agonist (e.g., or active metabolite(s) thereof) of about 0.1 ng/mL to about 0.5 ng/mL in the individual, wherein the minimum plasma concentration (Cₘᵢₙ) is determined at a time between 2 hours and 12 hours (or between 2 hours and 24 hours, or between 2 hours and 48 hours, or between 2 hours and 72 hours, or the like) after administration to the individual. In some embodiments, the method or composition provided herein provides a maximum plasma concentration of 5-HT receptor agonist (or active metabolite(s) thereof) is between about 2 ng/ml and 6 ng/ml. In more specific embodiments, the maximum plasma concentration of 5-HT receptor agonist (or active metabolite(s) thereof) is between about 2 ng/ml and about 5 ng/ml.

In specific embodiments, the formulations provided herein provide controlled release such that the minimum plasma concentration (Cₘᵢₙ) is determined at a time between 24 and 48 hours after administration. For example, if plasma concentration of the agent(s) continues to decline over time, then after 48 hours after administration, plasma levels of the agent(s) are at least the value indicated after 48 hours. In some embodiments, the minimum plasma concentration (Cₘᵢₙ) is determined at a time between 48 and 72 hours after administration. In certain embodiments, the minimum plasma concentration (Cₘᵢₙ) is determined at a time between 72 and 96 hours after administration. In some embodiments, the minimum plasma concentration (Cₘᵢₙ) is determined at a time between 96 to 120 hours after administration.

In more specific exemplary embodiments of pharmaceutical compositions (e.g., dosage forms) comprising a controlled release component, is a pharmaceutical composition comprising an extended release component and an immediate release component (e.g., for the 5-HT receptor agonist).

In certain preferred embodiments, the pharmaceutical composition or dosage form is formulated for oral administration. Other formulations are also contemplated herein, however, including intravenous formulations (e.g., osmotic pump), buccal formulations, nasal formulations, inhalation formulations, or any other suitable formulation.

In various embodiments herein, a composition (e.g., pharmaceutical composition, dosage form, combination or formulation) provided herein is administered at any frequency. For example, in some embodiments, a single dose is provided. In other embodiments, the composition is or is formulated to be administered to a subject in need thereof about once a week. In other embodiments, the composition is or is formulated to be administered to a subject in need thereof about once every two weeks. In various other embodiments, the composition is or is formulated for twice daily, once daily, twice weekly, thrice weekly, or the like administration.

In specific embodiments, the composition comprises an oral dosage form, the oral dosage form comprising a first (e.g., immediate release) component (e.g., layer, coating or core) and a second (e.g., extended release) component (e.g., layer, coating or core).

In some embodiments, the pharmaceutical composition comprises the 5HT receptor agonist or a pharmaceutically acceptable salt, solvate, metabolite, derivative, or prodrug thereof in an amount of about 0.0001 mg to about 600 mg (e.g. about 0.001 mg to about 100 mg, about 0.2 mg to about 25 mg, about 10 mg to about 50 mg, or the like). In some embodiments, the pharmaceutical composition comprises the 5HT receptor agonist or a pharmaceutically acceptable salt, solvate, metabolite, derivative, or prodrug thereof in an amount of about 0.1 mg to about 50 mg (e.g. about 0.1 mg to about 10 mg, about 0.2 mg to about 5 mg, about 10 mg to about 50 mg, or the like). In some embodiments, the pharmaceutical composition comprises the 5HT receptor agonist or a pharmaceutically acceptable salt, solvate, metabolite, derivative, or prodrug thereof in an amount of about 0.5 mg to about 5 mg. In some embodiments, the pharmaceutical composition comprises the 5HT receptor agonist or a pharmaceutically acceptable salt, solvate, metabolite, derivative, or prodrug thereof in an amount of about 0.5, 1, 1.5, 2, 2.5, 3, 3.5, 4, 4.5, or 5 mg. In some embodiments, the pharmaceutical composition comprises the 5HT receptor agonist or a pharmaceutically acceptable salt, solvate, metabolite, derivative, or prodrug thereof in an amount of about 1, 2, 3, 4, 5, 6, 7, 8, 9, 10, 11, 12, 13, 14, 15 mg. In some embodiments, the pharmaceutical composition comprises the 5HT receptor agonist or a pharmaceutically acceptable salt, solvate, metabolite, derivative, or prodrug thereof in an amount of about 1 mg to about 25, 2 mg to about 35 mg, about 5 mg to 30 mg, about 10 to 40 mg, about 25 mg to 35 mg. In some embodiments, the pharmaceutical composition comprises the 5HT receptor agonist or a pharmaceutically acceptable salt, solvate, metabolite, derivative, or prodrug thereof in an amount of about 16, 17, 18, 19, 20, 21, 22, 23, 24, 25, 26, 27, 28, 29, 30, 31, 32, 33, 34, 35, 36, 37, 38, 39, 40 mg. In some embodiments, the pharmaceutical composition is a low-dose pharmaceutical composition. In some embodiments, the pharmaceutical composition is an extended release composition.

In various embodiments, such a composition is formulated to have any of the components and/or features as described for a composition provided herein, such as described above. In some embodiments, such a composition comprises one or more pharmaceutically acceptable excipient (e.g., a filler, binder, suspending agent, disintegrant, lubricant, surfactant, preservative, flavoring agent, sweetener, or a combination of two or more thereof).

In certain embodiments, any composition provided herein is formulated and/or packaged to be repeatedly administered to a subject in need thereof about once a week (or more frequently, such as two or three times a week, daily, twice daily, or the like). In some embodiments, any composition provided herein is formulated and/or packaged to be repeatedly administered to a subject in need thereof about once every two weeks (or less frequently). In certain embodiments, any composition provided herein is formulated and/or packaged to be repeatedly administered to a subject in need thereof about once every month.

Provided in certain embodiments herein is a method comprising administering to the subject any composition (e.g., pharmaceutical combination, composition, dosage form, or formulation) provided herein. In specific embodiments, such a method comprises administering a therapeutically effective amount of one or more 5-HT receptor agonist as an extended release dosage form, such as providing a composition and/or effect described herein.

In specific embodiments, such a method is provided for of treating or managing a neurological condition or the symptoms thereof in a subject in need thereof (e.g., in a subject suffering from or susceptible to the neurological condition).

In various embodiments provided herein, the (e.g., one or more) 5-HT receptor agonist is any suitable 5-HT receptor agonist, such as a 5-HT2 receptor agonist, including free bases thereof, salts thereof, prodrugs thereof, metabolites thereof, and the like. In various embodiments provided herein, the (e.g., one or more) 5-HT receptor agonist is any suitable 5-HT receptor agonist, such as a 5-HT_{2A} receptor agonist, including free bases thereof, salts thereof, prodrugs thereof, metabolites thereof, and the like. In specific embodiments, the (e.g., one or more) 5-HT receptor agonist is psilocybin or psilocin, or a pharmaceutically acceptable salt, solvate, metabolite, derivative, or prodrug thereof.

In some embodiments, the therapeutically effective amount of 5-HT receptor agonist is an amount insufficient to provide a hallucinogenic experience (or other adverse effect) (e.g., in an average adult).

In some embodiments, the method provides a low-dose therapy. In specific embodiments, the low-dose therapy comprises administration of a such that following administration to an individual in need thereof, the low-dose therapy provides a maximum plasma concentration (Cₘₐₓ) of the 5-HT receptor agonist (e.g., or active metabolite(s) thereof) of less than 6 ng/mL in the individual in need thereof. In some embodiments, the low-dose therapy provides a plasma concentration (Cₘₐₓ) of the 5-HT receptor agonist (e.g., or active metabolite(s) thereof) of at least a therapeutic level (e.g. 0.01 ng/mL), but not higher than a concentration (e.g., Cₘₐₓ) of the 5-HT receptor agonist that produces an adverse event (e.g., a hallucinogenic threshold) in the individual in need thereof. In more specific embodiments, following administration to an individual in need thereof, the method provides a maximum plasma concentration (Cₘₐₓ) of the 5-HT receptor agonist (e.g., or active metabolite(s) thereof) of at least 0.5 ng/mL and less than 6 ng/mL in the individual in need thereof (e.g., about 1 ng/mL to about 5.5 ng/mL, about 2 ng/mL to about 5 ng/mL, or the like).

In some embodiments, the therapeutically effective amount of 5-HT receptor agonist or a pharmaceutically acceptable salt, solvate, metabolite, derivative, or prodrug thereof is provided to a subject in need thereof in an amount and/or formulation to provide a plasma concentration of (e.g., active form of the) 5-HT receptor agonist or a pharmaceutically acceptable salt, solvate, metabolite, derivative, or prodrug thereof of at least 0.1 ng/mL (e.g., at least 0.2 ng/mL, at least 0.3 ng/mL, at least 0.5 ng/mL, or the like) after at least 3 hours (e.g., at least 12 hours, at least 24 hours, at least 36 hours, at least 48 hours, at least 72 hours, at least 96 hours, at least 120 hours, at least 144 hours, or the like).

In various embodiments, the method comprises administration of a controlled release formulation, such that one or more of the agents is at least partially released in a controlled release manner. In specific embodiments, following administration to an individual in need thereof, the method provides a minimum plasma concentration (Cₘᵢₙ) of the 5-HT receptor agonist (e.g., or active metabolite(s) thereof) of about 0.1 ng/mL or more in the individual, wherein the minimum plasma concentration (Cₘᵢₙ) is determined at a time between 2 hours and 12 hours (or between 2 hours and 24 hours, or between 2 hours and 48 hours, or between 2 hours and 72 hours, or the like) after administration to the individual. In some embodiments, following administration to an individual in need thereof, the method provides a minimum plasma concentration (Cₘᵢₙ) of the 5-HT receptor agonist (e.g., or active metabolite(s) thereof) of about 0.1 ng/mL to about 0.5 ng/mL in the individual, wherein the minimum plasma concentration (Cₘᵢₙ) is determined at a time between 2 hours and 12 hours (or between 2 hours and 24 hours, or between 2 hours and 48 hours, or between 2 hours and 72 hours, or the like) after administration to the individual. In some embodiments, the method or composition provided herein provides a maximum plasma concentration of 5-HT receptor agonist (or active metabolite(s) thereof) is between about 2 ng/ml and 6 ng/ml. In more specific embodiments, the maximum plasma concentration of 5-HT receptor agonist (or active metabolite(s) thereof) is between about 2 ng/ml and about 5 ng/ml.

In specific embodiments, the formulations provided herein provide controlled release such that the minimum plasma concentration (Cₘᵢₙ) is determined at a time between 24 and 48 hours after administration. For example, if plasma concentration of the agent(s) continues to decline over time, then after 48 hours after administration, plasma levels of the agent(s) are at least the value indicated after 48 hours. In some embodiments, the minimum plasma concentration (Cₘᵢₙ) is determined at a time between 48 and 72 hours after administration. In certain embodiments, the minimum plasma concentration (Cₘᵢₙ) is determined at a time between 72 and 96 hours after administration. In some embodiments, the minimum plasma concentration (Cₘᵢₙ) is determined at a time between 96 to 120 hours after administration.

In certain preferred embodiments, the method comprises oral administration. Other routes of administration are also contemplated herein, however, including buccal administration, nasal administration, inhalation administration, or any other suitable administration routes. Administration provided herein is achieved in any suitable manner, such as via use of a spray, aerosol, mist, nebulae, ointment, cream, gel, paste, salve, solution, suspension, tincture, patch, or atomized vapor.

In various embodiments, methods provided herein are suitable for treating any suitable disorder, such as a neurological condition, such as a neurological disorder, or symptoms thereof. In specific embodiments, the neurological condition is a neurocognitive disorder. In some embodiments, symptoms of the neurological condition are physical, behavioral, emotional, mental or a combination thereof. In certain embodiments, the neurological condition is an addictive disorder (e.g., alcohol abuse, substance abuse, smoking, or obesity). In some embodiments, the neurological condition is an eating disorder or an auditory disorder. In certain embodiments, the neurological condition is pain (e.g., chronic pain). In some embodiments, the neurological condition is depression, bipolar disorder, post-traumatic stress disorder (PTSD), panic disorder, phobia, schizophrenia, psychopathy, or antisocial personality disorder. In certain embodiments, the neurological condition is an impulsive disorder. In some embodiments, the impulsive disorder is attention deficit hyperactivity disorder (ADHD), Tourette's syndrome or autism. In certain embodiments, the neurological condition is a compulsive disorder (e.g., obsessive compulsive disorder (OCD), gambling, or aberrant sexual behavior). In some embodiments, the neurological condition is a personality disorder (e.g., conduct disorder, antisocial personality, or aggressive behavior).

In various embodiments, the combination of agents is administered in any suitable formulation or form, such as in combination with one or more agents selected from the group consisting of surfactants, preservatives, flavoring agents, sweetening agents, and antifoaming agents. In certain embodiments, administration is via use of an oral formulation, a buccal formulation, a nasal formulation, or an inhalation formulation. In some embodiments, a spray, aerosol, mist, nebulae, ointment, cream, gel, paste, salve, solution, suspension, tincture, patch, or atomized vapor comprising one or both agent(s) is administered.

In certain embodiments, administration to a subject in need thereof occurs no more frequently than once a day (e.g., no more frequently than once every other day, no more frequently than once every third day, no more frequently than twice a week, no more frequently than once a week, no more frequently than once every two weeks, or the like). In some embodiments, administration to a subject in need thereof occurs once a day, every alternate day, three times a week, twice a week, once a week, every other week, two weeks per month, three weeks per month, once a month, twice a month or three times per month. In specific embodiments, administration is about once a day. In other specific embodiments, administration is about every alternate day. In still other specific embodiments, administration is about once a week. In yet other specific embodiments, administration is about once every two weeks or more.

In various embodiments, administration continues for any suitable length of time, such as at least 1 month, 2 months, 3 months, 4 months, 5 months, 6 months, 7 months, 8 months, 9 months, 10 months, 11 months, 12 months, 18 months, 2 years, or 3 years.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows the effects of a 5-HT receptor agonist provided herein (e.g., psilocybin) on locomotor activity and behavior signs in treated rats. A) Graph shows distance traveled (measured in cm) against doses of 5-HT receptor agonist ranging from 0.03-10 mg/kg. B) Graph shows effects of drug dosing on 5-HT_{2A}-related behaviors. The total number (N) of wet dog shakes (WDS) and back muscle contractions (BMC) is plotted against dose (mg/kg). Hatched shading indicated 5-HT receptor agonist doses which evoked statistically identical WDS and BMC behaviors as Vehicle.

FIG. 2 shows the effects of a 5-HT receptor agonist provided herein (e.g., psilocybin) on progressive ratio (PR) test in treated rats. FIG. 2A shows the number of lever presses by high and low responder rat subgroups of Study A across 5-HT receptor agonist doses of 0.05 mg/kg, 0.1 mg/kg and 0.2 mg/kg. FIG. 2B shows the number of break points by high and low responder rat subgroups of Study A across 5-HT receptor agonist doses of 0.05 mg/kg, 0.1 mg/kg and 0.2 mg/kg. FIG. 2C shows the number of lever presses by high and low responder rat subgroups of Study B across 5-HT receptor agonist doses of 0.025 mg/kg, 0.05 mg/kg, and 0.1 mg/kg. FIG. 2D shows the number of break points by high and low responder rat subgroups of Study B across 5-HT receptor agonist doses of 0.025 mg/kg, 0.05 mg/kg, and 0.1 mg/kg. High responders are defined as rats completing the highest tertile of lever presses in the baseline test; low responders are rats completing the bottom tertile of lever presses in the baseline test. Asterisk (*) indicates statistical significance between high and low responders.

FIG. 3 shows the effects of a 5-HT receptor agonist provided herein (e.g., psilocybin) on cognition in rats using the 5-choice serial reaction time task (5-CSRTT) with 5 second inter-trial interval. A) Graph shows pro-cognitive effects (measured as % Hit regarding nose-poke to stimulation location to collect a food reward) of 5-HT receptor agonist 0.05 mg/kg dose versus vehicle and 0.1 mg/kg dose across all rats. Asterisk (*) indicates statistical difference vs. vehicle. B) Graph shows pro-cognitive effects (measured as % Correct regarding accuracy in nose-poke) of 5-HT receptor agonist 0.05 mg/kg dose versus vehicle and 0.1 mg/kg dose across all rats. C) Graph shows pro-cognitive effect of two different doses of 5-HT receptor agonist on low performer subgroup (% Hit). Asterisk (*) indicates statistical difference vs. vehicle D) Graph shows effect of two different doses of 5-HT receptor agonist on low performer subgroup (% Correct).

FIG. 4 shows the effects of 5-HT receptor agonist on cognition in rats using 5-choice serial reaction time task (5-CSRTT) and evaluating premature responses (PREM) and perseverative responses (PSV). A) Graphs show increase in PREM and PSV responses under a 5 second inter-trial interval (ITI) which establishes a baseline (Base) and a 10 second ITI across 24 animals across two doses of a 5-HT receptor agonist provided herein (e.g., psilocybin) (0.05 mg/kg and 0.1 mg/kg). Standard deviation is indicated by error bars; asterisks (*) indicate significance vs. vehicle (P=0.05) using T-test. B) Graphs show effects of 5-HT receptor agonist on PREM and PSV in low performer and high performer subgroups. Standard error of the mean is indicated by error bars; asterisks (*) indicate significance vs. vehicle (P<0.01) using T-test.

FIG. 5 shows the blood plasma levels over time of psilocin in rats dosed with a 5-HT receptor agonist provided herein (e.g., psilocybin) at several dose levels: 0.05 mg/kg (e.g., Cₘₐₓ psilocin ˜6±2 ng/ml (after 30 mins)) or 0.1 mg/kg (e.g., Cₘₐₓ psilocin ˜12±3 ng/ml (after 30 mins)), 1 mg/kg (e.g., Cₘₐₓ psilocin ˜83±5 ng/ml (after 30 mins)), 10 mg/kg (e.g., Cₘₐₓ psilocin ˜1106±164 ng/ml (after 30 mins)).

FIG. 6 shows the effects of a 5-HT receptor agonist provided herein (e.g., psilocybin) on cognition in rats. A) Graph shows the lowest performing tertile (N=8) low attentive and potentially representative of a low attentive endophenotype of depression. B) Graph shows % hit score for low attentive rats. D) Graph shows a slower response speed for low attentive rats. Similar to the PR test, the effect of 0.05 and 0.1 mg/kg 5-HT receptor agonist on accuracy (% correct and % hit) in the 5CSRTT was observed to be strongly evident in the low attentive subgroup compared with vehicle C) graph and E) graph. Asterisk (*) indicates statistical significance vs. vehicle. 5-HT receptor agonist 0.05 mg/kg also increased response speed in the low attentive cohort compared with vehicle D) graph.

FIG. 7 shows psilocybin and psilocin exposure levels in rats after intraperitoneal (IP) injection (minipump infusion) of e.g., psilocybin (FIG. 7A). FIG. 7B shows the change in body weight of untreated and treated rats over a period of time (e.g., 12 days). FIG. 7C shows the change in body weight of placebo versus psilocybin treated rats over 12 days.

FIG. 8 shows the improvement in performance (e.g., motivation) of treated (e.g., with psilocybin) rats vs. untreated rats (FIG. 8A) over all test days (e.g., D1=test day 5, D2=test day 9, and D3=test day 12), with a significant improvement on D2, during challenging test conditions. FIG. 8B shows that the performance (e.g., motivation) of treated (e.g., with psilocybin) rats was improved over all test days (e.g., even the average of D1-D3).

FIG. 9 shows that 5-HT receptor agonist (e.g., psilocybin) does not increase the number of correct attempts during extended exposure of drug over a period of time under standard conditions.

FIG. 10 shows that rats with extended exposure to 5-HT receptor agonist (e.g., psilocybin) do not have an increase in premature behaviors over a period of time in standard conditions (e.g., FIG. 10A) nor challenge conditions (FIG. 10B).

### DETAILED DESCRIPTION

Provided in certain embodiments herein are methods, compositions, formulations and dosage forms comprising 5-HT (e.g., 5-HT_{2A}) receptor agonists, or the administration thereof.

### S-HT (or Serotonin) Receptors

The 5-HT (or serotonin) receptors area group of G protein-coupled receptors (GPCR) and ligand-gated ion channels. 5-HT is short for 5-hydroxy-tryptamine, the chemical name for serotonin.

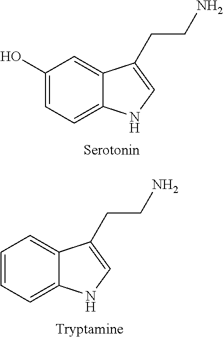 [SMILES: NCCc1c[nH]c2ccc(O)cc12 ; NCCc1c[nH]c2ccccc12]

The serotonin receptors are activated by serotonin, their natural ligand, and mediate both excitatory and inhibitory neurotransmission. They modulate the release of many neurotransmitters, including glutamate, GABA, dopamine, epinephrine/norepinephrine and acetylcholine, as well as many hormones, including oxytocin, prolactin, vasopressin, cortisol, corticotropin and substance P. The serotonin receptors influence various biological and neurological processes such as aggression, anxiety, appetite, cognition, learning, memory, mood, nausea, sleep, and thermoregulation.

The 5-HT receptors are divided into 7 families of G protein-coupled receptors. 5-HT₁, 5-HT₂, 5-HT₃ are the major families; the others, 5-HT₄, 5-HT₅, 5-HT₆ and 5-HT₇, for the most part, work in a similar fashion to either 5-HT₁ or 5-HT₂ receptors. The 5-HT receptors work with a G protein to modify an ion channel or membrane enzyme.

In certain embodiments, the 5-HT receptor agonist of a formulation, composition, method, or the like described herein is a 5-HT₁ agonist. 5-HT₁ receptors have strong binding affinity for serotonin. Typically, when serotonin binds to a 5-HT₁ receptor, a G-protein is activated, opening an ion channel and allowing potassium ions to exit the neuron. This generally causes the neuron to become more negatively charged, making it more difficult to trigger an action potential, i.e. serotonin binding to 5-HTₜ receptors is an inhibitory effect.

In some preferred embodiments, the 5-HT receptor agonist of a formulation, composition, method, or the like described herein is a 5-HT₂ agonist. In certain embodiments, the 5-HT₂ agonist has a relatively high affinity for 5-HT₂ receptors (e.g., relative to 5-HT₁ receptors and/or other 5-HT receptors, such as 5-HT₃, 5-HT₄, 5-HT₅, 5-HT₆, 5-HT₇, or all or some combination thereof, such as 2×, 3×, 5×, 10×, 20×, 50×, or the like greater affinity). 5-HT₂ receptors have weaker affinity for serotonin. As such, serotonin prefers to bind 5-HT₁ receptors, typically only binding 5-HT₂ receptors once the 5-HT₁ receptors are at least partially (or wholly) saturated. Serotonin binding of 5-HT₂ receptors typically activates a G-protein closing a potassium channel resulting in potassium ion build up. This generally causes depolarization, making it easier to reach the neuron's excitation threshold. Thus, when serotonin binds to 5-HT₂ receptors, it typically has an excitatory effect.

| Family | Type | Mechanism | Potential |
|---|---|---|---|
| 5-HT₁ | Protein coupled | Decreasing cellular levels of cAMP | Inhibitory |
| 5-HT₂ | Protein coupled | Increasing cellular levels of IP₃ and | Excitatory |
| | | DAG | |
| 5-HT₃ | Ligand-gated | Depolarizing plasma membrane | Excitatory |
| | Na⁺ and | | |
| | K⁺ cation | | |
| | channel | | |
| 5-HT₄ | Protein coupled | Increasing cellular levels of cAMP | Excitatory |
| 5-HT₅ | Protein coupled | Decreasing cellular levels of cAMP | Inhibitory |
| 5-HT₆ | Protein coupled | Increasing cellular levels of cAMP | Excitatory |
| 5-HT₇ | Protein coupled | Increasing cellular levels of cAMP | Excitatory |

The seven serotonin receptor families include fourteen receptor subtypes, distributed throughout the body as shown in the table below:

| | | Central | | | | |
|---|---|---|---|---|---|---|
| 5HT | Blood | nervous | Peripheral | | | Smooth |
| Receptor | vessels | system | nervous system | GI Tract | Platelets | Muscle |
| 1A | X | X | | | | |
| 1B | X | X | | | | |
| 1D | X | X | | | | |
| 1E | X | X | | | | |
| 1F | | X | | | | |
| 2A | X | X | X | X | X | X |
| 2B | X | X | X | X | X | X |
| 2C | X | X | X | X | X | X |
| 3 | | X | X | X | | |
| 4 | | X | X | X | | |
| 5A | | X | | | | |
| 5B | | | | | | |
| 6 | | X | | | | |
| 7 | X | X | | X | | |

### 5-HT₂ Receptors

In general, 5-HT₂ receptors are characterized by having lower affinity for serotonin (and other indolealkylamines), and are linked to the G_{q}/phospholipase C pathway of signal transduction. In various instances, such receptors mediate a variety of physiological and behavioral functions via three distinct subtypes: 5-HT_{2A}, 5-HT_{2B} and 5-HT_{2C}.

| Receptor | Physiological/behavioral function |
|---|---|
| 5-HT_{2A} | Addiction, Anxiety, Apathy, Appetite, |
| | Cognition, Depression, Imagination, |
| | Learning, Memory, Mood, Perception, Sexual Behavior, |
| | Sleep, Thermoregulation, Vasoconstriction |
| 5-HT_{2B} | Anxiety, Appetite, Cardiovascular Function, GI Motility, |
| | Sleep, Vasoconstriction |
| 5-HT_{2C} | Addiction, Anxiety, Appetite, GI Motility, |
| | Locomotion, Mood, Penile |
| | Erection, Sexual Behavior, Sleep, Thermoregulation, |
| | Vasoconstriction |

| Receptor | Uses of drugs that act on this receptor |
|---|---|
| 5-HT_{2A} | Antipsychotics , Psychedelics, Noradrenergic and Specific |
| | Serotonergic Antidepressants (NaSSAs), Sleeping aids |
| 5-HT_{2B} | Migraines |
| 5-HT_{2C} | Antidepressant, Orexigenic, Anorectic, Antipsychotic |

| Receptor | Agonists acting on receptor |
|---|---|
| 5-HT_{2A} | Bufotenin, Ergonovine, Lisuride, |
| | LSD, Mescaline, Myristicin, |
| | Psilocin, Psilocybin, DMT, DOM, PNU-22394, TFMPP, |
| | 25I-NBOMe, 2C-B, 5-MeO-DMT, BZP |
| 5-HT_{2B} | Fenfluramine, MDMA, Norfenfluramine, Methylphenidate |
| | 6-APB, BW-723C86, PNU-22394, Ro60-0175 |
| 5-HT_{2C} | Aripiprazole, Ergonovine, Lorcaserin, Trazodone |
| | PNU-22394, Ro60-0175, TFMPP, YM-348, A-372,159, |
| | AL-38022A |

5-HT_{2A} is an important excitatory serotonin receptor subtype. In some instances, physiological processes mediated by the receptor include, by way of non-limiting example:
central nervous system—neuronal excitation, behavioral effects, learning, anxiety, and pro-nociception
smooth muscle contraction (in bronchi and gastrointestinal tract)
vasoconstriction/vasodilation
platelet aggregation
role in memory and learning
anti-inflammatory activity
hormone (oxytocin, prolactin, ACTH, corticosterone, renin) regulation
mood regulation (depressed patients have more 5-HT_{2A} receptors than otherwise normal individuals implying S-HT_{2A} is involved in the pathogenesis of depression)

In some instances, agonism of 5-HT_{2A} agonism facilitates treatment or management of disorders involving cognitive function and social interaction, or the symptoms thereof, as evidenced by the extensive localization of the 5-HT_{2A} receptor in brain areas that mediate cognitive functions and social interaction. In some instances, disorders in which the 5-HT_{2A} receptor are involved include, but are not limited to schizophrenia, apathy, depression/suicide (e.g., low motivation), anxiety, obsessive compulsive disorders (OCD), bipolar disorders, attention deficit hyperactivity disorder (ADHD), eating disorders such as anorexia nervosa, autism and autism spectrum disorders, Asperger's, neuropsychiatric diseases and disorders, sexual disorders such as erectile dysfunction, neurodegenerative diseases, inflammatory diseases, autoimmune diseases, metabolic diseases such as obesity and diabetes, central nervous system disorders, peripheral nervous system disorders, Alzheimer's disease, snoring, sleep apnea (obstructive sleep apnea, central sleep apnea), insomnia, sleep deprivation, restless legs syndrome, parasomnia, nightmares, night terrors, sleepwalking, hypersomnia (daytime sleepiness), narcolepsy and pain.

Any suitable 5-HT (e.g., 5-HT₂, such as 5-HT_{2A}) agonist is utilized in any composition, formulation, method, therapy, or the like described herein. In some preferred embodiments, the 5-HT receptor agonist of a formulation, composition, method, or the like described herein is a 5-HT_{2A} agonist. In certain embodiments, the 5-HT_{2A} agonist has a relatively high affinity for 5-HT_{2A} receptors (e.g., relative to 5-HT1, 5-HT₃, 5-HT₄, 5-HT₅, 5-HT₆, 5-HT₇, 5-HT_{2B}, 5-HT_{2C}, or all or some combination thereof, such as 2×, 3×, 5×, 10×, 20×, 50×, or the like greater affinity). In some instances, 5-HT_{2A} agonists increase dopamine levels in the prefrontal cortex. In certain embodiments, the 5-HT_{2A} agonist provided herein is one of the following classes of 5-HT_{2A} agonists: the ergolines, tryptamines and phenethylamines.

### Ergolines

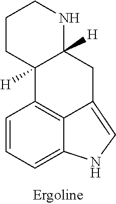 [SMILES: c1cc2c3c(c[nH]c3c1)C[C@H]1NCCC[C@H]21]

In specific embodiments, a 5-HT (e.g., 5-HT_{2A}) receptor agonist utilized herein is an ergoline. In some instances, ergonovine and ergotamine, synthetic derivatives include the oxytocic methergine, the anti-migraine drugs dihydroergotamine and methysergide, hydergine (a mixture of dihydroergotoxine mesylates, INN: ergoline mesylates), and bromocriptine. In certain instances, synthetic ergolines include pergolide and lisuride.

In certain instances, the ergoline is an ergoline derivative, such as a lysergic acid amide or a peptide alkaloid, such as described below. In some instances, the ergoline is a clavine (examples include festuclavine, fumigaclavine A, fumigaclavine B and fumigaclavine C) and other derivatives that do not fall into these categories, such as cabergoline, pergolide, lisuride.

### Lysergic Acid Amides

Exemplary lysergic acid amides include Ergine (LSA, D-lysergic acid amide), Ergonovine (ergobasine), Methergine (ME-277), Methysergide (UML-491), LSD (D-lysergic acid diethylamide), LSH (D-lysergic acid α-hydroxyethylamide). The table below summarizes their structural formula and relationships.

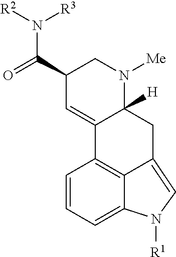

| Name | R¹ | R² | R³ |
|---|---|---|---|
| Ergine | H | H | H |
| Ergonovine | H | CH(CH₃)CH₂OH | H |
| Methergine | H | CH(CH₂CH₃)CH₂OH | H |
| Methysergide | CH₃ | CH(CH₂CH₃)CH₂OH | H |
| LSD | H | CH₂CH₃ | CH₂CH₃ |
| LSH | H | CH(OH)CH₃ | H |

### Peptide Alkaloids

Exemplary peptide alkaloids include, peptide ergot alkaloids (ergopeptines or ergopeptides), which are ergoline derivatives containing a tripeptide structure (attached at the same position as the amide group of the lysergic acid derivatives) comprising proline and two other α-amino acids. Examples include:
Ergotoxines (valine at R²)—Ergocristine, Ergocornine, α-Ergocryptine, β-Ergocryptine
Ergotamines (alanine at R²)—Ergotamine, Ergovaline, α-Ergosine, β-Ergosine.

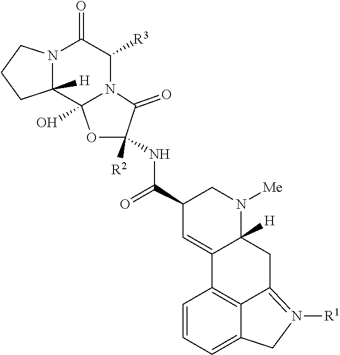

| | | | | R³ Amino |
|---|---|---|---|---|
| Name | R¹ | R² | R³ | acid |
| Ergocristine | | CH(CH₃)₂ | benzyl | Phenylalanine |
| Ergocornine | | CH(CH₃)₂ | CH(CH₃)₂ | Valine |
| α-Ergocryptine | | CH(CH₃)₂ | CH₂CH(CH₃)₂ | Leucine |
| β-Ergocryptine | | CH(CH₃)₂ | (S)-CH(CH₃)CH₂CH₃ | Isoleucine |
| Ergotamine | | CH₃ | benzyl | Phenylalanine |
| Ergovaline | | CH₃ | CH(CH₃)₂ | Valine |
| α-Ergosine | | CH₃ | CH₂CH(CH₃)₂ | Leucine |
| β-Ergosine | | CH₃ | (S)-CH(CH₃)CH₂CH₃ | Isoleucine |
| Bromocriptine | Br | CH(CH₃)₂ | CH₂CH(CH₃)₂ | Leucine |

### Tryptamines

Tryptamine (2-(1H-Indol-3-yl)ethanamine) comprises an indole ring, attached to an aminoethylene group; substituted tryptamines are substituted with any suitable group, such as being modified on the indole ring (R¹, R²), the ethylene chain (R³) and/or on the amino group (R⁴, R⁵), as illustrated below, and are collectively referred to herein as tryptamines. Examples of tryptamines include serotonin, melatonin, psilocybin and N,N-Dimethyltryptamine. Additionally, the tryptamine structure may comprise part of a more complex compound, for example: LSD, ibogaine, mitragynine, yohimbine, etc.

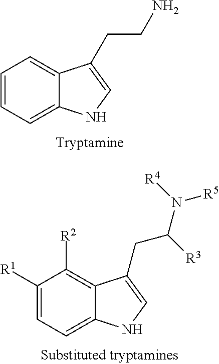

Examples of naturally occurring substituted tryptamines include, by way of non-limiting example:

| Short/Common | | | | | | |
|---|---|---|---|---|---|---|
| Name | Full Name | R¹ | R² | R³ | R⁴ | R⁵ |
| Tryptamine | 3-(2-aminoethyl)indole | H | H | H | H | H |
| | 2-(1H-indol-3- | | | | | |
| | yl)ethanamine | | | | | |
| Bufotenin | 5-hydroxy-N,N- | OH | H | H | CH₃ | CH₃ |
| | dimethyltryptamine | | | | | |
| Nω- | 5-hydroxy-N- | OH | H | H | CH₃ | H |
| Methylserotonin | methyltryptamine | | | | | |
| (norbufotenin) | | | | | | |
| Serotonin | 5-hydroxytryptamine | OH | H | H | H | H |
| NMT | N-methyltryptamine | H | H | H | H | CH₃ |
| 5-MeO-NMT | 5-methoxy-N- | OCH₃ | H | H | CH₃ | H |
| | methyltryptamine | | | | | |
| DMT | N,N-dimethyltryptamine | H | H | H | CH₃ | CH₃ |
| 5-Bromo-DMT | 5-bromo-N,N- | Br | H | H | CH₃ | CH₃ |
| | dimethyltryptamine | | | | | |
| 5-MeO-DMT | 5-methoxy-N,N- | OCH₃ | H | H | CH₃ | CH₃ |
| | dimethyltryptamine | | | | | |
| Melatonin | 5-methoxy-N- | OCH₃ | H | H | C(O)CH₃ | H |
| | acetyltryptamine | | | | | |
| N- | 5-hydroxy-N- | OH | H | H | C(O)CH₃ | H |
| Acetylserotonin | acetyltryptamine | | | | | |
| Norbaeocystin | 4-phosphoryloxy- | H | OPO₃H₂ | H | H | H |
| | tryptamine | | | | | |
| Baeocystin | 4-phosphoryloxy-N-methyl- | H | OPO₃H₂ | H | CH₃ | H |
| | tryptamine | | | | | |
| Psilocybin | 4-phosphoryloxy-N,N- | H | PO₄ | H | CH₃ | CH₃ |
| | dimethyltryptamine | | | | | |
| Psilocin | 4-hydroxy-N,N- | H | OH | H | CH₃ | CH₃ |
| | dimethyltryptamine | | | | | |
| Tryptophan | α-carboxyltryptamine | H | H | COOH | H | H |

Examples of synthetic substituted tryptamines include, by way of non-limiting example:

| Short Name | Name | R¹ | R² | R³ | R⁴ | R⁵ |
|---|---|---|---|---|---|---|
| ET | α-ethyltryptamine | H | H | CH₂CH₃ | H | H |
| αMT | α-methyltryptamine | H | H | CH₃ | H | H |
| DALT | N,N-diallyltryptamine | H | H | H | H₂C═CH—CH₂ | H₂C═CH—CH₂ |
| DET | N,N-diethyltryptamine | H | H | H | CH₂CH₃ | CH₂CH₃ |
| DiPT | N,N-dilsopropyltryptamine | H | H | H | CH(CH₃)₂ | CH(CH₃)₂ |
| DPT | N,N-dipropyltryptamine | H | H | H | CH₂CH₂CH₃ | CH₂CH₂CH₃ |
| 5-MeO-αMT | 5-methoxy-α- | OCH₃ | H | CH₃ | H | H |
| | methyltryptamine | | | | | |
| 5-MeO- | 5-methoxy-N,N- | OCH₃ | H | H | H₂C═CH—CH₂ | H₂C═CH—CH₂ |
| DALT | diallyltryptamine | | | | | |
| 5-MeO- | 5-methoxy-N-Methyl-N- | OCH₃ | H | H | H₂C═CH—CH₂ | CH₃ |
| MALT | allyltryptamine | | | | | |
| 4-HO-DET | 4-hydroxy-N,N- | H | OH | H | CH₂CH₃ | CH₂CH₃ |
| | diethyltryptamine | | | | | |
| 4-AcO-DMT | 4-acetoxy-N,N- | H | OCOCH₃ | H | CH₃ | CH₃ |
| | dimethyltryptamine | | | | | |
| 4-HO-MET | 4-hydroxy-N-methyl-N- | H | OH | H | CH₃ | CH₂CH₃ |
| | ethyltryptamine | | | | | |
| 4-HO-DIPT | 4-hydroxy-N,N- | H | OH | H | CH(CH₃)₂ | CH(CH₃)₂ |
| | diisopropyltryptamine | | | | | |
| 5-MeO-DIPT | 5-methoxy-N,N- | OCH₃ | H | H | CH(CH₃)₂ | CH(CH₃)₂ |
| | diisopropyltryptamine | | | | | |
| 5-MeO- | 5-methoxy-N,N- | OCH₃ | H | H | CH₃ | CH(CH₃)₂ |
| MiPT | methylisopropyltryptamine | | | | | |
| 4-HO-MIPT | 4-hydroxy-N-isopropyl-N- | H | OH | H | CH(CH₃)₂ | CH₃ |
| | methyltryptamine | | | | | |
| Sumatriptan | 5-(methylamino | | | | | |
| | sulfonylmethylene)-N,N- | CH₂SO₃NHCH₃ | H | H | CH₃ | CH₃ |
| | dimethyltryptamine | | | | | |
| Zolmitriptan | 5-(4-(S)-1,3-oxazolidin-2- | CHNHC(O)OCH₂ | H | H | CH₃ | CH₃ |
| | one)-N,N-dimethyl- | | | | | |
| | tryptamine | | | | | |

### Phenethylamines

Phenethylamine comprises a phenyl ring attached to an aminoethylene group; substituted phenethylamines are optionally substituted in any suitable manner, such as they are optionally modified by substitution on the phenyl ring (R¹, R², R³, R⁴ and/or R⁵), the ethylene chain (R⁶ and/or R⁷) and/or on the amino group (R⁸, and/or R⁹), such as illustrated below.

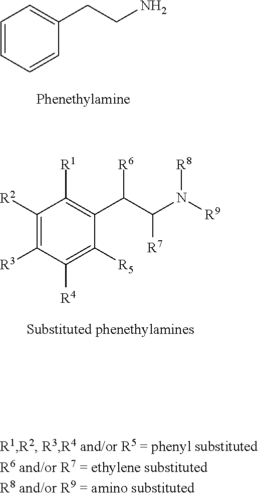

Examples of phenethylamines include, but are not limited those presented in the table below:

| | | Substitution | | | |
|---|---|---|---|---|---|
| Short Name | Full Name | Amino | Ethylene | Phenyl | Biological activity |
| Amphetamine | α-methylphenethylamine | | X | | Stimulant |
| β-Methyl | β-methylphenethylamme | | X | | Stimulant |
| phenethylamine | | | | | |
| Mephedrone | 4-methylmethcathinone | X | X | X | Stimulant |
| Ethcathinone | N-ethylcathinone | X | X | | Stimulant |
| Ephedrine/ | N-methyl-β- | X | X | | Stimulant; |
| Pseudoephedrine | hydroxyamphetamine | | | | decongestant |
| Methamphetamine | N-methylamphetamine | X | X | | Stimulant; neurotoxin |
| Phentermine | α-methylamphetamine | | | X | Stimulant, anorectic |
| Ortetamine | 2-methylamphetamine | | X | X | Stimulant, anorectic |
| Amfepramone | N-diethyl-β- | X | X | | Anorectic |
| (diethylpropion) | ketoamphetamine | | | | |
| Phenylephrine | β,3-dihydroxy-N- | X | X | X | Decongestant |
| | methylphenethylamine | | | | |
| Methylphenidate | N,α-butylene-β-methoxy | X | X | | Stimulant; NDRI |
| | carbonylphenethylamine | | | | |
| Dopamine | 3,4- | | | X | Catecholamine |
| | dihydroxyphenethylamine | | | | neurotransmitter |
| 6- | 2,4,5-trihydroxy | | | X | Neurotoxic agent |
| Hydroxydopamine | phenethylamine | | | | |
| Epinephrine | β-3,4-trihydroxy-N- | X | X | X | Catecholamine |
| (Adrenaline) | methylphenethylamine | | | | neurotransmitter |
| Norepinephrine | β-3,4-trihydroxy | | X | X | Catecholamine |
| (Noradrenaline) | phenethylamine | | | | neurotransmitter |
| para-Octopamine | β-4-dihydroxy | | X | X | Trace aminergic α- |
| | phenethylamine | | | | adrenoceptor agonist |
| Salbutamol | β-4-dihydroxy-3- | X | X | X | Short-action β2- |
| | hydroxymethyl-N-tert- | | | | adrenergic agonist |
| | butylphenethylamine | | | | |
| N-Methyl | N-methyphenethylamine | X | | | Amphetamine isomer |
| phenethylamine | | | | | |
| Cathine | d-β- | | X | | Releasing agent |
| | hydroxyamphetamine | | | | |
| Cathinone | β-ketoamphetamine | | X | | Releasing agent |
| Methcathinone | N-methylcathinone | X | X | | Releasing agent |
| Bupropion | 3-chloro-N-tert-butyl-β- | X | X | X | NDRI |
| | ketoamphetamine | | | | |
| Norfenturamine | 3-trifluoromethyl- | | X | X | SSRA |
| | amphetamine | | | | |
| Fenfluramine | 3-trifluoromethyl-N- | X | X | X | SSRA |
| | ethylamphetamine | | | | |
| Mescaline | 3,4,5-trimethoxy | | | X | Psychedelic |
| | phenethylamine | | | | |
| Proscaline | 2-(3,5-dimethoxy-4- | | | X | Psychedelic |
| | propoxyphenyl)ethanamine | | | | |
| Metaescaline | 2-(3-ethoxy-4,5- | | | X | Psychedelic |
| | dimethoxyphenyl)ethan- | | | | |
| | amine | | | | |
| Allylescaline | 4-Allyloxy-3,5- | | | X | Psychedelic |
| | dimethyloxy | | | | |
| | phenylethylamine | | | | |
| Methallylescaline | 4-Methallyloxy-3,5- | | | X | Psychedelic |
| | dimethoxyphenethylamine | | | | |
| Asymbescaline | 3,4-Diethoxy-5- | | | X | Psychedelic |
| | methoxyphenethylamine | | | | |

In certain embodiments, described herein are pharmaceutical compositions, comprising 5-HT receptor agonist, or pharmaceutically acceptable salt, solvate, metabolite, derivative, or prodrug thereof, such agents being collectively referred to herein as 5-HT receptor agonist agents. 5-HT receptor agonist agents are also disclosed in PCT/IB2020/000052, filed Jan. 29, 2020, which is incorporated herein by reference in its entirety. In some instances, the pharmaceutical formulations of 5-HT receptor agonist, or pharmaceutically acceptable salt, solvate, metabolite, derivative, or prodrug thereof, have enhanced bioavailability and efficacy, have a lower administration dose, a lower cytotoxicity, and/or have decreased side effects.

In various embodiments provided herein, any suitable route of administration is contemplated. In specific embodiments, the composition is formulated for oral, buccal, nasal or inhalation administration. In certain embodiments, the composition is an oral, buccal, nasal or inhalation composition.

In specific embodiments, the composition further comprises any suitable (e.g., pharmaceutically acceptable) excipients and/or additives, such as surfactants, preservatives, flavoring agents, sweetening agents, or anti-foaming agents.

Any suitable composition, formulation, or dosage form is contemplated herein. In certain embodiments, the composition, formulation, or dosage form is an oral composition, formulation or dosage form. In some specific embodiments, the pharmaceutically acceptable excipient is selected from the group consisting of fillers, binders, suspending agents, disintegrants, lubricants, and combinations thereof.

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a”, “an”, and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

The term “comprising” (and related terms such as “comprise” or “comprises” or “having” or “including”), as used herein, generally means that at least all of the listed elements must exist but other elements not recited may also be present. The term “consists of,” as used herein generally means that only the listed elements may be present. The term “consists essentially of,” as used herein generally means that the listed elements and other elements may be present. The term “comprising” is not intended to exclude that in other embodiments, for example, an embodiment of any composition of matter, composition, method, or process, or the like, described herein, may “consist of” or “consist essentially of” the described features.

The section headings used herein are for organizational purposes only and are not to be construed as limiting the subject matter disclosed.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error. In some embodiments, about means±5% of the amount disclosed.

The term “controlled release dosage form” refers to a drug's release characteristic of time course and/or location chosen to accomplish therapeutic or convenience objectives not offered by conventional immediate-release dosage forms. The rate of release of the active drug from a controlled release dosage form is controlled by features of the dosage form and/or in combination with physiologic or environmental conditions rather than by physiologic or environmental conditions alone. The controlled release dosage forms are used to maintain drug plasma levels within the therapeutic window (e.g., for an extended period of time). The controlled release dosage forms of certain embodiments attempt to deliver therapeutically effective amounts of active drug as a once-daily dose so that the ratio Cₘₐₓ/Cₘᵢₙ in the plasma at steady state is less than the therapeutic index, and to maintain drug levels at constant effective levels to provide a therapeutic benefit over a period of time (e.g. 24-hour period). In certain embodiments controlled release dosage forms provide a substantially constant or gradually decreasing rate of drug release so as to provide plasma levels which remain substantially invariant with time. In some embodiments, the drug releases is invariant over an extended period of time (e.g., 12-hour period, 24-hour period, 48-hour period, or 72-hour period) In certain embodiments, controlled release dosage forms are designed to provide a quick increase in the plasma concentration of the drug which remains substantially constant within the therapeutic range of the drug for a period of time (e.g. 24-hour period). Alternatively, in some other embodiments controlled release dosage forms are designed to provide a quick increase in the plasma concentration of the drug, which although might not remain constant, declines at a rate such that the plasma concentration remains within the therapeutic range for a period of time (e.g. 24-hour period).

The term “immediate-release” dosage form refers to the release of an active agent substantially immediately upon administration. For example, immediate-release includes but not limited to contact with gastric juices and results in substantially complete dissolution within about 1 hour. Immediate-release components might also be referred to as instant release. When used in association with the dissolution profiles discussed herein, the term “immediate-release” refers to that portion of a dosage form disclosed herein which delivers active agent over a period of time less than 1 hour.

The terms “coating composition”, “coat composition”, “coating solution”, “coat solution”, “coating suspension”, and “coat suspension” as used herein are used interchangeably and are defined to mean a mixture of excipients that is used to create a controlled release coating. The coating composition is applied onto a 5-HT receptor agonist or a pharmaceutically acceptable salt, solvate, metabolite, derivative, or prodrug thereof core to form an intermediate coating, and the intermediate coating is cured to form the controlled release coating.

The terms “effective amount” or “pharmaceutically effective amount” or “therapeutically effective amount” refer to a nontoxic but sufficient amount of the agent to provide the desired biological, therapeutic, and/or prophylactic result. That result might be reduction and/or alleviation of the signs, symptoms, or causes of a disease, or any other desired alteration of a biological system. For example, an “effective amount” for therapeutic uses is the amount of a 5-HT receptor agonist or a pharmaceutically acceptable salt, solvate, metabolite, derivative, or prodrug thereof as disclosed herein per se or a composition comprising a 5-HT receptor agonist or a pharmaceutically acceptable salt, solvate, metabolite, derivative, or prodrug thereof as disclosed herein required to provide a clinically significant decrease in a disease. An appropriate effective amount in any individual case might be determined by one of ordinary skill in the art using routine experimentation.

In some instances, the term “low dose,” as used herein, refers to an amount of a therapeutic agent (e.g. a 5-HT receptor agonist or a pharmaceutically acceptable salt, solvate, metabolite, derivative, or prodrug thereof), which is sufficient to provide the desired biological, therapeutic, and/or prophylactic result, while insufficient to induce an undesired effect (e.g. such as a hallucinogenic experience, a perturbation in the user's sense of reality or perceptions).

The term “5-HT receptor agonist agent” refers to a 5-HT receptor agonist as a free base or a derivate or analog thereof. Included in the term are salts, solvates, metabolites, prodrugs, isomers, tautomers, isotopic derivatives, and the like, of a 5-HT receptor agonist. In some embodiments, the derivates, analogs, salts, solvates, metabolites, prodrugs, isomers, tautomers, isotopic derivatives, etc are pharmaceutically acceptable derivates, analogs, salts, solvates, metabolites, prodrugs, isomers, tautomers, isotopic derivatives of a 5-HT receptor agonist.

The term “pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich:Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound described herein with an acid to provide a “pharmaceutically acceptable acid addition salt.” In some embodiments, the compound described herein (i.e. free base form) is basic and is reacted with an organic acid or an inorganic acid. Inorganic acids include, but are not limited to, hydrochloric acid, hydrobromic acid, metaphosphoric acid, nitric acid, phosphoric acid, and sulfuric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; monomethyl fumarate, naphthalene-1,5-disulfonic acid; naphthalene-2-sulfonic acid; nicotinic acid; oleic acid; oxalic acid; palmitic acid; pamoic acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, a compound described herein is prepared as a chloride salt, sulfate salt, bromide salt, mesylate salt, maleate salt, citrate salt or phosphate salt.

In some embodiments, pharmaceutically acceptable salts are obtained by reacting a compound described herein with a base to provide a “pharmaceutically acceptable base addition salt”. In some embodiments, the compound described herein is acidic and is reacted with a base. In such situations, an acidic proton of the compound described herein is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

It should be understood that a reference to a pharmaceutically acceptable salt includes the solvent addition forms, i.e. solvates. In some embodiments, solvates contain either stoichiometric or non-stoichiometric amounts of a solvent, and are formed during the process of isolating or purifying the compound with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

The methods and formulations described herein include the use of N-oxides (if appropriate), crystalline forms (also known as polymorphs), or pharmaceutically acceptable salts of compounds described herein, as well as active metabolites of these compounds having the same type of activity.

In some embodiments, sites on the organic radicals (e.g. alkyl groups, aromatic rings) of compounds described herein are susceptible to various metabolic reactions. Incorporation of appropriate substituents on the organic radicals will reduce, minimize or eliminate this metabolic pathway. In specific embodiments, the appropriate substituent to decrease or eliminate the susceptibility of the aromatic ring to metabolic reactions is, by way of example only, a halogen, deuterium, an alkyl group, a haloalkyl group, or a deuteroalkyl group.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as, for example, ²H, ³H, ¹³C, ¹⁴C, ¹⁵N, ¹⁸O, ¹⁷O, ³⁵S, ¹⁸F, ³⁶Cl. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements. In some embodiments, one or more hydrogen atoms of the compounds described herein is replaced with deuterium.

In some embodiments, the compounds described herein possess one or more stereocenters and each stereocenter exists independently in either the R or S configuration. The compounds presented herein include all diastereomeric, enantiomeric, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. The prodrug might be a substrate fora transporter. Further or alternatively, the prodrug also has improved solubility in pharmaceutical compositions over the parent drug. In some embodiments, the design of a prodrug increases the effective water solubility. An example, without limitation, of a prodrug is a compound described herein, which is administered as an ester (the “prodrug”) but then is metabolically hydrolyzed to provide the active entity. A further example of a prodrug is a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

Prodrugs of the compounds described herein include, but are not limited to, esters, ethers, carbonates, thiocarbonates, N-acyl derivatives, N-acyloxyalkyl derivatives, quaternary derivatives of tertiary amines, N-Mannich bases, Schiff bases, amino acid conjugates, phosphate esters, and sulfonate esters. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. In some embodiments, a hydroxyl group in the compounds disclosed herein is used to form a prodrug, wherein the hydroxyl group is incorporated into an acyloxyalkyl ester, alkoxycarbonyloxyalkyl ester, alkyl ester, aryl ester, phosphate ester, sugar ester, ether, and the like. In some embodiments, a hydroxyl group in the compounds disclosed herein is a prodrug wherein the hydroxyl is then metabolized in vivo to provide a carboxylic acid group. In some embodiments, a carboxyl group is used to provide an ester or amide (i.e. the prodrug), which is then metabolized in vivo to provide a carboxylic acid group. In some embodiments, compounds described herein are prepared as alkyl ester prodrugs.

Prodrug forms of the herein described compounds, wherein the prodrug is metabolized in vivo to produce a compound described herein as set forth herein are included within the scope of the claims. In some cases, some of the herein-described compounds is a prodrug for another derivative or active compound.

In additional or further embodiments, the compounds described herein are metabolized upon administration to an organism in need to produce a metabolite that is then used to produce a desired effect, including a desired therapeutic effect.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes might produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Metabolites of the compounds disclosed herein are optionally identified either by administration of compounds to a host and analysis of tissue samples from the host, or by incubation of compounds with hepatic cells in vitro and analysis of the resulting compounds.

A compound is “dissolved” when it is “in solution”, and does not spontaneously come out of solution to from a separate phase. In order to be dissolved, the compound need not dissociate completely on a molecular level, but must remain in solution so as to be effective in treatment of a disease or condition. A dissolved compound might be present in a micellar, emulsified, or liposomal form.

“Solubility” generally means the amount of a compound dissolved in a solvent. Suitable solvents include aqueous and non-aqueous solvents.

“Poor solubility” means a small amount of compound dissolved in a solvent. Poor solubility is not an absolute term, but depends on the amount of the compound that is needed for effective treatment of a disease or condition. A compound will be poorly soluble if its solubility is lower than is desired in order for an effective treatment of a disease or condition.

“Enhanced solubility” means higher solubility than for a 5-HT receptor agonist or a pharmaceutically acceptable salt, solvate, metabolite, derivative, or prodrug thereof alone. Enhanced solubility in water can be useful because many bodily fluids such as blood are water based (aqueous) and therefore, a more water soluble drug might have higher bioavailability. While the exact solubility of a compound in pure water is not the same as in an aqueous solution such as blood, a composition's solubility in pure water is often a good indication of solubility in other aqueous solutions.

A “tautomer” refers to a proton shift from one atom of a molecule to another atom of the same molecule. The compounds presented herein might exist as tautomers. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH. Some examples of tautomeric interconversions include:

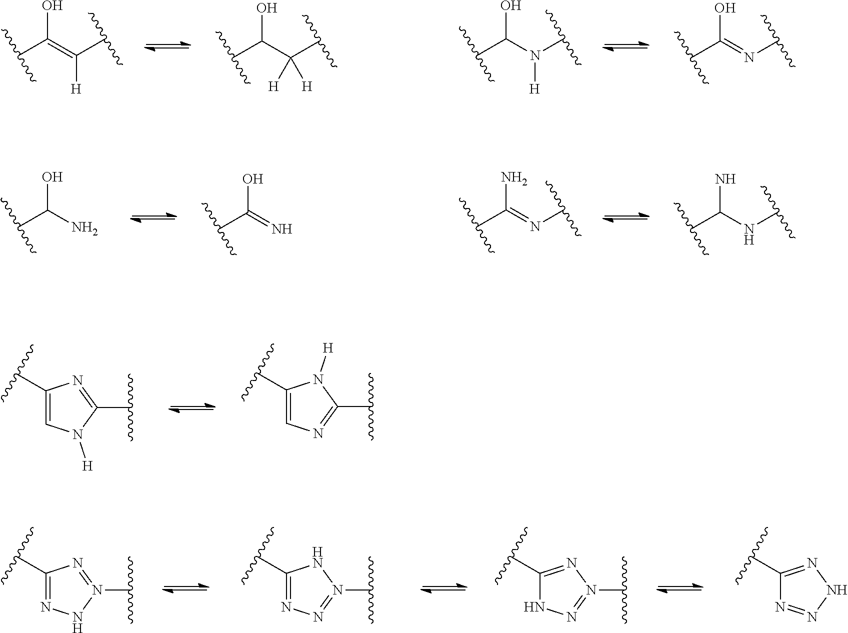

The term “taste masking agents” when used herein refers to taste receptor blockers, compounds which mask the chalkiness, grittiness, dryness and/or astringent taste properties of an active compound, compounds which reduce throat catch as well as compounds which add a flavour.

The term “enhancers” when used herein refers to agents which work to increase membrane permeability and/or work to increase the solubility of a particular active. Both issues can be pivotal to the properties of the formulation. The following are examples. Chelators: EDTA, citric acid, sodium salicylate, methoxysalicylates. (See Senel & Hincal: JCR 72 2001133-144; Malhalingam et al: AAPS Pharmascitech 2007 (8) vol 3 Article 55). Surfactants: sodium lauryl sulphate, polyoxyethylene, POE-9-laurylether, POE-20-cetylether, benzalkonium chloride, 23-lauryl ether, cetylpyridinium chloride, cetyltrimethyl ammonium bromide, amphoteric and cationic surfactants. Membrane disrupting compounds such as powdered alcohols (eg menthol and ethanol), and compounds such as lipophilic enhancers which are safe to be used orally. (Nicolazzo, Reid and Finnin J Pharmaceutical Sciences Vol 93, No 8 Aug. 2004 2054-2063). Fatty and other acids: oleic acid, capric acid, lauric acid, lauric acid/propylene glycol, methyloleate, ysophosphatidylcholine, phosphatidylcholine (Sudhakar et al JCR 114 (2006) 15-40), oleic acid co-delivered with PEG 200, (Lee and Kellaway Int J Pharmaceutics 204 (2000) 137-144). Lysalbinic acid (Starokadomdkyy & Dubey Int J Pharmaceutics 308 (2006) 149-154). Non-surfactants such as unsaturated cyclic ureas. Others: glucosaminoglycans (GAGs), aprotinin, azone, cyclodextrin, dextran sulfate, curcumin, menthol, polysorbate 80, sulfoxides and various alkyl glycosides. Chitosan-4-thiobutylamide, chitosan-4-thiobutylamide/GSH, chitosan-cysteine, chitosan-(85% degree N-deacetylation), poly(acrylic acid)-homocysteine, polycarbophil-cysteine, polycarbophil-cysteine/GSH, chitosan-4-thioethylamide/GSH, chitosan-4-thioglycholic acid. Hyaluronic acid in 3 MW's (Sandri et al: J Pharmacy and Pharmacology 2004, 56: 1083-1090.) Bile Salts (Dihydroxy and Trihydroxy), sodium glycocholate, sodium deoxycholate, sodium taurocholate, sodium glycodeoxycholate, sodium taurodeoxycholate (Artusi et al: Int J Pharmaceutics 250 (2003) 203-213). Propanolol hydrochloride (Akbari et al: Il Farmaco 59 (2004)155-161).

The term “treating” and its grammatical equivalents as used herein include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding the fact that the patient might still be afflicted with the underlying disorder. For prophylactic benefit, a method might be performed on, or a composition might be administered to a patient at risk of developing a disease, or to a patient reporting one or more of the physiological symptoms of such conditions, even though a diagnosis of the condition might not have been made.

While generally high drug solubility is desired, is would be appreciated by a person of ordinary skill in the art that there are other considerations in creating a pharmaceutical composition such as viscosity, stability, potential toxicity, etc. that might result a composition with lower solubility being more desirable for a particular therapy or delivery method as long as the amount of available drug is enough for the application. Pharmaceutical compositions disclosed herein provide the ability to optimize these factors.

### Formulations

In some embodiments, described herein are compositions and formulations comprising a 5-HT receptor agonist active ingredient. In some embodiments, described herein are compositions and formulations comprising a 5-HT receptor agonist or pharmaceutically acceptable salt, solvate, metabolite, derivative, or prodrug thereof. In some embodiments, the formulation comprising 5-HT receptor agonist, or pharmaceutically acceptable salt, solvate, metabolite, derivative, or prodrug thereof, has: an enhanced bioavailability and efficacy, a lower administration dose, a lower cytotoxicity, decreased side effects, or the like.

In some embodiments, the formulation comprises one or more 5-HT receptor agonist, or a pharmaceutically acceptable salt, solvate, metabolite, derivative, or prodrug thereof. In some embodiments, the one or more 5-HT receptor agonist, or a pharmaceutically acceptable salt, solvate, metabolite, derivative, or prodrug thereof is a 5HT_{2A} and/or a 5HT_{2C} receptor agonist. In some embodiments, the one or more 5-HT receptor agonist, or a pharmaceutically acceptable salt, solvate, metabolite, derivative, or prodrug thereof is a 5HT_{2A} receptor agonist. In some embodiments, the one or more 5-HT receptor agonist, or a pharmaceutically acceptable salt, solvate, metabolite, derivative, or prodrug thereof is a 5HT_{2C} receptor agonist. In some embodiments, the one or more 5-HT receptor agonist, or a pharmaceutically acceptable salt, solvate, metabolite, derivative, or prodrug thereof is a 5HT_{2A} and a 5HT_{2C} receptor agonist. In some embodiments, the one or more 5-HT receptor agonist, or a pharmaceutically acceptable salt, solvate, metabolite, derivative, or prodrug thereof weakly or negligibly binds the 5HT_{2B} receptor.

In some embodiments, the one or more 5-HT receptor agonist, or the pharmaceutically acceptable salt, solvate, metabolite, derivative, or prodrug thereof, is a hallucinogenic compound (e.g., wherein the hallucinogenic compound produces a hallucinogenic effect (e.g., an adverse event, a clinically important effect (e.g., clinically important impairment of the individual, altered (e.g., visual, auditory, body, time and space) perception, altered cognition, impaired attention, drowsiness, and/or confusion)) in the individual in need thereof at or above the hallucinogenic threshold (e.g., at or above a Cₘₐₓ above the hallucinogenic effective threshold)).

In some embodiments, the one or more 5-HT receptor agonist is selected from the group consisting of LSD, dimethyltryptamine (DMT), psilocybin, and psilocin, or a pharmaceutically acceptable salt, solvate, metabolite, derivative, or prodrug thereof. In some embodiments, the one or more 5-HT receptor agonist is psilocybin or psilocin, or a pharmaceutically acceptable salt, solvate, metabolite, derivative, or prodrug thereof. In some embodiments, the one or more 5-HT receptor agonist is psilocin.

In some embodiments, provided herein is a formulation configured to maintain a level of an active 5-HT receptor agonist at or above a minimum therapeutically effective threshold of the active 5-HT receptor agonist in an individual (e.g., in a biological sample (e.g., serum, plasma, or whole blood) of the individual) for more than or equal to two hours (e.g., more than 2 hours, more than 12 hours, more than 1 day, more than 7 days, or more than 14 days). In some embodiments, provided herein is a formulation configured to maintain a level of an active 5-HT receptor agonist below a hallucinogenic threshold (e.g., below a threshold that produces an adverse event (e.g., a clinically important effect (e.g., clinically important impairment of the individual), altered (e.g., visual, auditory, body, time and space) perception, altered cognition, impaired attention, drowsiness, confusion, or the like) of the active 5-HT receptor agonist in an individual (e.g., in a biological sample (e.g., serum, plasma, or whole blood) of the individual) for more than or equal to two hours (e.g., more than 2 hours, more than 12 hours, more than 1 day, more than 7 days, or more than 14 days).

In some embodiments, provided herein is a formulation configured to maintain a level of an active 5-HT receptor agonist (i) at or above a minimum therapeutically effective threshold and (ii) below a hallucinogenic threshold (e.g., below a threshold that produces an adverse event (e.g., a clinically important effect (e.g., clinically important impairment of the individual), altered (e.g., visual, auditory, body, time and space) perception, altered cognition, impaired attention, drowsiness, confusion, or the like) of the active 5-HT receptor agonist in the individual (e.g., in a biological sample (e.g., serum, plasma, or whole blood) of the individual) for more than or equal to two hours (e.g., more than 2 hours, more than 12 hours, more than 1 day, more than 7 days, or more than 14 days).

In some embodiments, the repeated exposure (e.g., a repeat dosing regimen provided herein) of the composition provided herein is provided or maintained at approximately the same dose and/or concentration of the 5HT receptor agonist (e.g., as an initial dose or subsequent dose of the 5HT receptor agonist). In some embodiments, the individual is administered a repeat dosing regimen. In some embodiments, the repeat dosing regimen comprises at least one ascending dose of the 5HT receptor agonist. In some embodiments, the repeat dosing regimen comprises at least one descending dose of the 5HT receptor agonist. In some embodiments, the repeat dosing regimen (e.g., regardless of the concentration (e.g. ascending, descending, or same concentration)) comprises a dose of the 5-HT receptor agonist that is (i) at or above a minimum therapeutically effective threshold and (ii) below a hallucinogenic threshold of the active 5-HT receptor agonist in the individual. In some embodiments, the single (e.g., full) dose is 15 mg (e.g., administered in a 24-hr period (e.g., as 2 doses of, for example, 7.5 mg, 3 doses of 5 mg or 4 dose of 3.75 mg)). In some embodiments, the single (e.g., full) dose amount is 10 mg (e.g., administered in a 24-hr period (e.g., as 2 doses of 5 mg, 3 doses of 3.33 mg or 4 dose of 2.5 mg)). In one embodiment, the single (e.g., full) dose amount is 7.5 mg (e.g., administered in a 24-hr period (e.g., as 2 doses of 3.75 mg, 3 doses of 2.5 mg or 4 dose of 1.88 mg)). In some instances, a single (e.g., full) dose (e.g. 100, 90, 80, 75, 70, 65, 60, 55, 50, 45, 40, 35, 30, 25, 24, 23, 22, 21, 20, 19, 18, 17, 16, 15, 14, 13, 12, 11, 10, 9, 8, 7, 6, 5, 4, 3, 2, 1, 0.05 mg) provided herein is administered more frequently (e.g. as a half dose bi-daily, a third of a dose three times a day, or a fourth of a dose four times a day) or as an extended release, such as, for example, to avoid an adverse event (e.g., hallucinations), such as, associated with the entire amount of the single (e.g., full) dose (e.g., taken at once).

In some embodiments, provided herein is a formulation configured to release the active 5-HT receptor agonist at a dose sufficient to provide a Cₘₐₓ below the hallucinogenic effective threshold of the active 5-HT receptor agonist and a Cₘᵢₙ of at least the therapeutically effective threshold of the active 5-HT receptor agonist in the individual.

In some embodiments, provided herein is a formulation configured to provide a maximum plasma concentration (Cₘₐₓ) of the active 5-HT receptor agonist of about 0.1 ng/mL to about 6 ng/mL (e.g., about 0.5 ng/mL to about 6 ng/mL, about 1 ng/mL to about 5.5 ng/mL, about 2 ng/mL to about 5 ng/mL, or the like) in the individual. In further embodiments, the low-dose pharmaceutical composition is a pharmaceutical composition (e.g., dosage form) such that following administration to an individual in need thereof, the low-dose pharmaceutical composition provides a plasma concentration (Cₘₐₓ) of the 5-HT receptor agonist (e.g., or active metabolite(s) thereof) of at least a therapeutic level (e.g. 0.01 ng/mL), but not higher than a concentration (e.g., Cₘₐₓ) of the 5-HT receptor agonist that produces an adverse event (e.g., a hallucinogenic threshold) in the individual in need thereof.

In some embodiments, provided herein is a formulation configured to provide a plasma concentration of the active 5-HT receptor agonist of at least 0.1 ng/mL (e.g., at least 0.2 ng/mL, at least 0.3 ng/mL, at least 0.5 ng/mL, or the like) in the individual for at least 3 hours (e.g., at least 12 hours, at least 24 hours, at least 36 hours, at least 48 hours, at least 72 hours, at least 96 hours, at least 120 hours, at least 144 hours, or the like).

In some embodiments, the composition or formulation is an oral formulation. In some instances, the oral formulation comprising 5-HT receptor agonist, or pharmaceutically acceptable salt, solvate, metabolite, derivative, or prodrug thereof, has: an enhanced bioavailability and efficacy, a lower administration dose, a lower cytotoxicity, and decreased side effects.

In some embodiments, the pharmaceutically acceptable excipient is selected from the group consisting of fillers, binders, suspending agents, disintegrants, lubricants, and combinations thereof.

In some embodiments, the composition or formulation (e.g. oral composition or formulation) comprises a filler. In some embodiments, the amount of the filler is from about 10% to about 20% by weight. In some embodiments, the amount of the filler is from about 10% to about 40% by weight. In some embodiments, the amount of the filler is from about 20% to about 40% by weight. In some embodiments, the amount the filler is about 10% w/w, about 11% w/w, about 12% w/w, about 13% w/w, about 14% w/w, about 15% w/w, about 16% w/w, about 17% w/w, a bout 18% w/w, about 19% w/w, a bout 20% w/w, about 21% w/w, about 22% w/w, about 23% w/w, about 24% w/w, about 25% w/w, about 26% w/w, about 27% w/w, about 28% w/w, about 29% w/w, about 30% w/w, about 31% w/w, about 32% w/w, about 33% w/w, about 34% w/w, about 35% w/w, about 36% w/w, about 37% w/w, about 38% w/w, about 39% w/w, or about 40% w/w.

In some embodiments, the composition or formulation (e.g. oral composition or formulation) comprises a binder. In some embodiments, the amount of the binder is from about 5% to about 15% by weight. In some embodiments, the amount of the binder is from about 5% to about 25% by weight. In some embodiments, the amount of the binder is from about 15% to about 25% by weight. In some embodiments, the amount of the binder is about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, about 19% w/w, about 20% w/w, about 21% w/w, about 22% w/w, about 23% w/w, about 24% w/w, or about 25% w/w.

In some embodiments, the composition or formulation (e.g. oral composition or formulation) comprises a suspending agent. In some embodiments, the amount of the suspending agent is from about 2% to about 3% by weight. In some embodiments, the amount of the suspending agent is from about 2% to about 4% by weight. In some embodiments, the amount of the suspending agent is from about 1% to about 5% by weight. In some embodiments, the amount of the suspending agent is about 1% w/w, about 1.1% w/w, about 1.2% w/w, about 1.3% w/w, about 1.4% w/w, about 1.5% w/w, about 1.6% w/w, about 1.7% w/w, about 1.8% w/w, about 1.9% w/w, about 2% w/w, about 2.1% w/w, about 2.2% w/w, about 2.3% w/w, about 2.4% w/w, about 2.5% w/w, about 2.6% w/w, about 2.7% w/w, about 2.8% w/w, about 2.9% w/w, about 3% w/w, about 3.1% w/w, about 3.2% w/w, about 3.3% w/w, about 3.4% w/w, about 3.5% w/w, about 3.6% w/w, about 3.7% w/w, about 3.8% w/w, about 3.9% w/w, about 4% w/w, about 4.1% w/w, about 4.2% w/w, about 4.3% w/w, about 4.4% w/w, about 4.5% w/w, about 4.6% w/w, about 4.7% w/w, about 4.8% w/w, about 4.9% w/w, or about 5% w/w.

In some embodiments, the composition or formulation (e.g. oral composition or formulation) comprises a disintegrant. In some embodiments, the amount of the disintegrant is from about 2% to about 3% by weight. In some embodiments, the amount of the disintegrant is from about 2% to about 4% by weight. In some embodiments, the amount of the disintegrant is from about 1% to about 5% by weight. In some embodiments, the amount of the disintegrant is about 1% w/w, about 1.1% w/w, about 1.2% w/w, about 1.3% w/w, about 1.4% w/w, about 1.5% w/w, about 1.6% w/w, about 1.7% w/w, about 1.8% w/w, about 1.9% w/w, about 2% w/w, about 2.1% w/w, about 2.2% w/w, about 2.3% w/w, about 2.4% w/w, about 2.5% w/w, about 2.6% w/w, about 2.7% w/w, about 2.8% w/w, about 2.9% w/w, about 3% w/w, about 3.1% w/w, about 3.2% w/w, about 3.3% w/w, about 3.4% w/w, about 3.5% w/w, about 3.6% w/w, about 3.7% w/w, about 3.8% w/w, about 3.9% w/w, about 4% w/w, about 4.1% w/w, about 4.2% w/w, about 4.3% w/w, about 4.4% w/w, about 4.5% w/w, about 4.6% w/w, about 4.7% w/w, about 4.8% w/w, about 4.9% w/w, or about 5% w/w.

In some embodiments, the composition or formulation (e.g. oral composition or formulation) comprises a lubricant. In some embodiments, the amount of the lubricant is from about 2% to about 3% by weight. In some embodiments, the amount of the lubricant is from about 2% to about 4% by weight. In some embodiments, the amount of the lubricant t is from about 1% to about 5% by weight. In some embodiments, the amount of the lubricant is about 1% w/w, about 1.1% w/w, about 1.2% w/w, about 1.3% w/w, about 1.4% w/w, about 1.5% w/w, about 1.6% w/w, about 1.7% w/w, about 1.8% w/w, about 1.9% w/w, about 2% w/w, about 2.1% w/w, about 2.2% w/w, about 2.3% w/w, about 2.4% w/w, about 2.5% w/w, about 2.6% w/w, about 2.7% w/w, about 2.8% w/w, about 2.9% w/w, about 3% w/w, about 3.1% w/w, about 3.2% w/w, about 3.3% w/w, about 3.4% w/w, about 3.5% w/w, about 3.6% w/w, about 3.7% w/w, about 3.8% w/w, about 3.9% w/w, about 4% w/w, about 4.1% w/w, about 4.2% w/w, about 4.3% w/w, about 4.4% w/w, about 4.5% w/w, about 4.6% w/w, about 4.7% w/w, about 4.8% w/w, about 4.9% w/w, or about 5% w/w.

In some embodiments, the composition or formulation (e.g. oral composition or formulation) comprises a surfactant. In some embodiments, the amount of the surfactant is from about 0.1% to about 2% by weight. In some embodiments, the amount of the surfactant is from about 0.1% to about 5% by weight. In some embodiments, the amount of the surfactant is from about 1% to about 15% by weight. In some embodiments, the amount of the surfactant is about 0.5% w/w, about 0.6% w/w, about 0.7% w/w, about 0.8% w/w, about 0.9% w/w, about 1% w/w, about 1.1% w/w, about 1.2% w/w, about 1.3% w/w, about 1.4% w/w, about 1.5% w/w, about 1.6% w/w, about 1.7% w/w, about 1.8% w/w, about 1.9% w/w, about 2% w/w, about 2.1% w/w, about 2.2% w/w, about 2.3% w/w, about 2.4% w/w, about 2.5% w/w, about 2.6% w/w, about 2.7% w/w, about 2.8% w/w, about 2.9% w/w, about 3% w/w, about 3.1% w/w, about 3.2% w/w, about 3.3% w/w, about 3.4% w/w, about 3.5% w/w, about 3.6% w/w, about 3.7% w/w, about 3.8% w/w, about 3.9% w/w, about 4% w/w, about 4.1% w/w, about 4.2% w/w, about 4.3% w/w, about 4.4% w/w, about 4.5% w/w, about 4.6% w/w, about 4.7% w/w, about 4.8% w/w, about 4.9% w/w, about 5%, about 6%, about 7%, about 8%, about 9%, about 10%, about 11%, about 12%, about 13%, about 14%, or about 15%.

In some embodiments, the amount of the surfactant is from about 0.5% to about 5% by weight. In some embodiments, the amount of the surfactant is about 0.5% by weight. In some embodiments, the amount of the surfactant is about 1% by weight. In some embodiments, the amount of the surfactant is about 2% by weight. In some embodiments, the amount the surfactant is about 3% by weight. In some embodiments, the amount of the surfactant is about 4% by weight. In some embodiments, the amount of the surfactant is from about 7% to about 15% by weight. In some embodiments, the amount of the surfactant is from about 0.5% to about 2% by weight.

In some embodiments, provided herein is a controlled release formulation that releases an active 5-HT receptor agonist. In some embodiments, the controlled release formulation releases the active 5-HT receptor agonist at a Cₘₐₓ below the hallucinogenic effective threshold and a Cₘᵢₙ of at least the therapeutically effective threshold.

In some embodiments, the controlled release formulation comprises an extended release component (e.g., that releases the active 5-HT receptor agonist (e.g., at a Cₘₐₓ below the hallucinogenic effective threshold and a Cₘᵢₙ of at least the therapeutically effective threshold)). In some embodiments, the controlled release formulation releases the active 5-HT receptor agonist in the individual in need thereof for a period of at least two hours. In some embodiments, the controlled release formulation releases the active 5-HT receptor agonist in the individual in need thereof for a period of at least one day. In some embodiments, the controlled release formulation is released in the individual in need thereof for a period of two hours to one week.

In some embodiments, the controlled release formulation is an oral formulation, a dermal formulation, a buccal formulation, a nasal formulation, or an inhalation formulation. In some embodiments, the oral formulation is in a solid form or a liquid form.

In some embodiments, at least one (e.g. controlled-release) coating (e.g. at least partially, or wholly) surrounds the core of the oral dosage form. In certain embodiments the controlled release coating is a stable controlled release monolithic coating that is formed by a process that comprises coating the core with a coating composition to form a coated core with an intermediate coating, and curing the coated core to form the stable controlled release coating. In at least one embodiment the coating composition comprises an aqueous dispersion of a neutral ester copolymer without any functional groups, a poly-glycol having a melting point of at least 55° C., and one or more second pharmaceutically acceptable excipients. In some instances, the curing is conducted at a temperature at least equal to or greater than the melting point of the poly-glycol. In at least one embodiment the stable controlled release coating comprises a neutral ester copolymer without any functional groups, a poly-glycol having a melting point of at least 55° C., and one or more second pharmaceutically acceptable excipients.

In some embodiments, a composition, formulation, core or coating described herein (e.g. a stable controlled release coating) hydrates when placed into water. In some embodiments, the dosage form (e.g. that is coated with the controlled release coating) floats in water. In some embodiments, the (e.g. controlled release) dosage form, upon oral administration to an individual, provides controlled release of an effective amount of the active drug to at least one region of the patient's upper gastrointestinal tract (e.g. the stomach).

In some embodiments, any composition, formulation, core or coating described herein (e.g. controlled release coating) is formed by a process that does not involve the use of an organic solvent. In such embodiments the controlled release coating composition is aqueous-based and not solvent-based (termed “AQ” in certain examples of dosage forms coated with the aqueous-based controlled release coating). In some embodiments, the composition, formulation, core or coating described herein (e.g. controlled release coating) is formed by a process that are solvent based (e.g. “PharmaPASS™” composition).

In some embodiments, the coating formulation is used to coat a variety of 5-HT receptor agonist cores and might be adjusted to obtain a desired drug release profile. The length and time for the delay is controlled by rate of hydration and the thickness of the coat. The drug release rate subsequent to the delay is determined by the thickness and permeability of the hydrated coat. Thus, it is possible to regulate the rate of hydration and permeability of the coat so that the desired controlled release drug profile might be achieved. There is no preferred coat thickness, as this will depend on the drug being used in the core and also the controlled release profile desired. Other parameters in combination with the thickness of the coat include varying the concentrations of some of the ingredients of the stable coat composition and/or varying the curing temperature and length of curing the coated tablet cores. The skilled artisan will know which parameters or combination of parameters to change for a desired controlled release profile.

In some embodiments, a composition or formulation (e.g. oral dosage form) provided herein comprises an immediate-release coating (e.g. providing immediate release of a 5-HT receptor agonist). In some embodiments, the immediate-release coating comprises a 5-HT receptor agonist. In some embodiments, the immediate-release coating comprises more than one 5-HT receptor agonist. In further embodiments, the immediate release coating comprises a pharmaceutically acceptable excipient.

In some embodiments, the immediate-release formulation provided herein releases the therapeutically effective amount of one or more 5-hydroxytryptamine (5-HT) receptor agonist) provides an active 5HT receptor agonist within 2 hours or less (1.5 hours or less, 1 hour or less, 30 minutes or less, or the like).

In some embodiments, an (e.g. therapeutically) effective amount of the immediate release active agent in immediate release form is coated onto the formulations provided herein. For example, in some instances where the extended release of a 5HT receptor agonist or a pharmaceutically acceptable salt, solvate, metabolite, derivative, or prodrug thereof from the formulation is due to a controlled release coating, the immediate release layer of the additional agent would be overcoated on top of the controlled release coating. In some embodiments, the immediate release layer of the additional agent is coated onto the surface of substrates, wherein the 5-HT receptor agonist or a pharmaceutically acceptable salt, solvate, metabolite, derivative, or prodrug thereof is incorporated in a controlled release matrix. Where a plurality of the sustained release substrates comprising an effective unit dose of the 5-HT receptor agonist or a pharmaceutically acceptable salt, solvate, metabolite, derivative, or prodrug thereof (e.g. multiparticulate systems including pellets, spheres, beads and the like) are incorporated into a hard gelatin capsule, the side effect-reducing compound might be incorporated into the gelatin capsule via inclusion of the sufficient amount of immediate release antihistamine or antiemetic as a powder or granulate within the capsule. Alternatively, the gelatin capsule itself is optionally coated with an immediate release layer of the additional agent.

In some embodiments, a composition or formulation provided herein (e.g. a formulation comprising an immediate release component and a controlled release component) is in the form of a bi-layered tablet, such as comprising a first layer and a second layer. In some embodiments, the first layer is an immediate release layer and/or the second layer is a controlled release layer. The first (or top) or immediate release layer comprises a first active agent, such as a 5-HT receptor agonist and/or another agent, such as an agent selected from analgesics, antitussives, antihistamines, antiemetics, and stimulants. In some instances, the second or controlled release layer comprises a second drug, such as a 5-HT receptor agonist. In some embodiments, the second drug is a 5-HT receptor agonist or a pharmaceutically acceptable salt, solvate, metabolite, derivative, or prodrug thereof. In some embodiments, the second drug is a formulation of a 5-HT receptor agonist or a pharmaceutically acceptable salt, solvate, metabolite, derivative, or prodrug thereof described herein. In some embodiments, the bi-layered tablet provides a plasma concentration within the therapeutic range of the second drug over a period which is coextensive with at least about 70% of the period (e.g., 12 hours) within which the bi-layered tablet provides a plasma concentration within the therapeutic range of the first drug.

In some embodiments, provided herein is a formulation comprising a controlled release component (e.g., that releases the active 5-HT receptor agonist (e.g., at a Cₘₐₓ below the hallucinogenic effective threshold and a Cₘᵢₙ of at least the therapeutically effective threshold)) and an immediate release component (e.g., that releases the active 5-HT receptor agonist (e.g., at a Cₘₐₓ below the hallucinogenic effective threshold and a Cₘᵢₙ of at least the therapeutically effective threshold)). In some embodiments, the controlled release component is an extended release component (e.g., that releases the active 5-HT receptor agonist (e.g., at a Cₘₐₓ below the hallucinogenic effective threshold and a Cₘᵢₙ of at least the therapeutically effective threshold)). In some embodiments, provided herein is a controlled release formulation that comprises an extended release component (e.g., that releases the active 5-HT receptor agonist (e.g., at a Cₘₐₓ below the hallucinogenic effective threshold and a Cₘᵢₙ of at least the therapeutically effective threshold)) and an immediate release component (e.g., that releases the active 5-HT receptor agonist (e.g., at a Cₘₐₓ below the hallucinogenic effective threshold and a Cₘᵢₙ of at least the therapeutically effective threshold)). In some embodiments, the immediate release component is an immediate-release coating (e.g., providing immediate release of the active 5-HT receptor agonist). In some embodiments, the immediate-release coating surrounds the controlled release component (e.g., wherein the immediate-release coating and the controlled release component each release the active 5-HT receptor agonist).

In some embodiments, the formulation comprises more than or equal to 1% weight by weight (w/w) (e.g., more than 1% w/w, more than 5% w/w, more than 10% w/w, more than 25% w/w, more than 50% w/w, more than 75% w/w, or more than 90% w/w) of the immediate release component. In some embodiments, the formulation comprises less than or equal to 90% w/w (e.g., less than 90% w/w, less than 75% w/w, less than 50% w/w, less than 25% w/w, less than 10% w/w, less than 5% w/w, or less than 1% w/w) of the immediate release component. In some embodiments, the formulation comprises from about 1% w/w to about 90% w/w (e.g., from about 5% w/w to about 85% w/w, from about 10% w/w to about 50% w/w, or from about 20% w/w to about 30% w/w) of the immediate release component.

In some embodiments, the formulation comprises more than or equal to 1% weight by weight (w/w) (e.g., more than 1% w/w, more than 5% w/w, more than 10% w/w, more than 25% w/w, more than 50% w/w, more than 75% w/w, or more than 90% w/w) of the controlled release component. In some embodiments, the formulation comprises less than or equal to 95% w/w (e.g., less than 90% w/w, less than 75% w/w, less than 50% w/w, less than 25% w/w, less than 10% w/w, less than 5% w/w, or less than 1% w/w) of the controlled release component. In some embodiments, the formulation comprises from about 1% w/w to about 95% w/w (e.g., from about 15% w/w to about 95% w/w, from about 50% w/w to about 90% w/w, or from about 70% w/w to about 80% w/w) of the controlled release component.

In some embodiments, the formulation comprises less of the immediate release component than the controlled release component.

In some embodiments, the formulation comprises more of the immediate release component than the controlled release component.

In some embodiments, the composition or formulation is or comprises a bi-layer formulation (e.g. oral dosage form). In some instances, the bi-layer formulation comprising 5-HT receptor agonist, or pharmaceutically acceptable salt, solvate, metabolite, derivative, or prodrug thereof, has: an enhanced bioavailability and efficacy, a lower administration dose, a lower cytotoxicity, and/or decreased side effects.

In some embodiments, the bi-layer formulation is an oral dosage form comprising an (e.g. immediate release or controlled release) top layer or coating and a controlled release core, such as wherein at least one of (i) the top layer or coating, or (ii) the controlled release core comprise a 5-HT (e.g. 5-HT_{2A}) receptor agonist. In specific instances, the (e.g. immediate release or controlled release) top layer or coating and the controlled release core both comprise a 5-HT (e.g. 5-HT_{2A}) receptor agonist, the core and coating 5HT receptor agonist being the same or different. Additional agents are contemplated in either or both the (i) top layer or coating and (ii) the core, such as any additional agent described herein (e.g. stimulant, an antihistamine, an antiemetic, an antidepressant, an anti-inflammatory, a growth factor, a lithium compound, resveratrol, phosphatidylcholine, curcumin, magnesium, melatonin, pregnenolone, ginseng, tryptophan, lysergic acid diethylamide, a 5HT receptor antagonist, or any combination thereof).

In some embodiments, provided herein is a controlled release formulation or composition (e.g. an oral dosage form, or a core of an oral dosage form, or a layer of an oral dosage form, such as a tablet). In certain embodiments, the controlled release formulation or composition is coated or layered with another composition or formulation. In some instances, the controlled release formulation or composition is coated or layered with an immediate and/or controlled release coating or layer, such as described herein. In specific embodiments, a controlled release composition or formulation provided herein comprises a 5-HT receptor agonist formulation comprising a controlled release matrix and (e.g., from about 0.1 to about 50 mg, about 10 mg to about 50 mg, about 0.1 mg to about 10 mg, about 0.2 mg to about 5 mg, or about 0.1 mg to about 2 mg, or about 1 mg to about 15 mg) of a 5-HT receptor agonist or a pharmaceutically acceptable salt, solvate, metabolite, derivative, or prodrug thereof. In some embodiments, the controlled and/or immediate release coating or layer comprises a first active agent, such as a 5-HT receptor agonist. In some embodiments, the pharmaceutical composition is a controlled release matrix or an immediate release coating and comprises the 5HT receptor agonist or a pharmaceutically acceptable salt, solvate, metabolite, derivative, or prodrug thereof in an amount of about 0.5 mg to about 5 mg. In some embodiments, the pharmaceutical composition is a controlled release matrix or an immediate release coating and comprises the 5HT receptor agonist or a pharmaceutically acceptable salt, solvate, metabolite, derivative, or prodrug thereof in an amount of about 0.5, 1, 1.5, 2, 2.5, 3, 3.5, 4, 4.5, or 5 mg.

In some embodiments, the pharmaceutical composition is a controlled release matrix or an immediate release coating and comprises the 5HT receptor agonist or a pharmaceutically acceptable salt, solvate, metabolite, derivative, or prodrug thereof in an amount of about 0.001 mg to about 600 mg (e.g. about 0.01 mg to about 100 mg, about 0.2 mg to about 25 mg, about 10 mg to about 50 mg, or the like). In some embodiments, the pharmaceutical composition is a controlled release matrix or an immediate release coating and comprises the 5HT receptor agonist or a pharmaceutically acceptable salt, solvate, metabolite, derivative, or prodrug thereof in an amount of about 0.1 mg to about 50 mg (e.g. about 0.1 mg to about 10 mg, about 0.2 mg to about 5 mg, about 10 mg to about 50 mg, or the like). In some embodiments, the pharmaceutical composition is a controlled release matrix or an immediate release coating and comprises the 5HT receptor agonist or a pharmaceutically acceptable salt, solvate, metabolite, derivative, or prodrug thereof in an amount of about 0.5 mg to about 5 mg. In some embodiments, the pharmaceutical composition is a controlled release matrix or an immediate release coating and comprises the 5HT receptor agonist or a pharmaceutically acceptable salt, solvate, metabolite, derivative, or prodrug thereof in an amount of about 0.5, 1, 1.5, 2, 2.5, 3, 3.5, 4, 4.5, or 5 mg. In some embodiments, the pharmaceutical composition is a controlled release matrix or an immediate release coating and comprises the 5HT receptor agonist or a pharmaceutically acceptable salt, solvate, metabolite, derivative, or prodrug thereof in an amount of about 1, 2, 3, 4, 5, 6, 7, 8, 9, 10, 11, 12, 13, 14, 15 mg. In some embodiments, the pharmaceutical composition is a controlled release matrix or an immediate release coating and comprises the 5HT receptor agonist or a pharmaceutically acceptable salt, solvate, metabolite, derivative, or prodrug thereof in an amount of about 1 mg to about 25, 2 mg to about 35 mg, about 5 mg to 30 mg, about 10 to 40 mg, about 25 mg to 35 mg. In some embodiments, the pharmaceutical composition is a controlled release matrix or an immediate release coating and comprises the 5HT receptor agonist or a pharmaceutically acceptable salt, solvate, metabolite, derivative, or prodrug thereof in an amount of about 16, 17, 18, 19, 20, 21, 22, 23, 24, 25, 26, 27, 28, 29, 30, 31, 32, 33, 34, 35, 36, 37, 38, 39, 40 mg. In some embodiments, the pharmaceutical composition is a low-dose pharmaceutical composition. In some embodiments, the pharmaceutical composition is an extended release composition.

Active agents are released from a controlled release matrix in any suitable manner. Two exemplary mechanisms of release of the active agent(s) from a controlled release matrix include diffusion and/or degradation. Generally, diffusion occurs when the bioactive agent is released either through pores in the polymer matrix or by passing between polymer chains of the matrix. Typically, in a diffusion system, the bioactive agent might be dispersed throughout the matrix, or localized within a reservoir adjacent to or within the matrix. In some embodiments, the controlled release formulation utilizes a reservoir system, which typically comprises a reservoir of bioactive agent, for example, solid drug, dilute solution, or highly concentrated drug solution within a polymer matrix is surrounded by a controlled release material through which the bioactive agent is able to diffuse. Generally, in a degradable system, the bioactive agent is released as the matrix is degraded in vivo. In some instances, bioactive agent is released by a combination of such mechanisms. In some embodiment of the controlled release matrix described herein, the release of the bioactive agent is driven by a combination of both diffusion and degradation. In certain instances, the release rate is controlled by varying the drug to polymer ratio (e.g. a higher drug concentration tends to result in a faster rate of release) and/or by varying the chemistry of polymeric matrix (e.g. inclusion of polymers having a glass transition temperature (Tg) of less than about 40° C. or less than about 0° C. would tend to result in a faster elution rate than polymers with Tgs greater than 40° C., polymers that absorb water tend to elute drug more quickly than more hydrophobic polymers that do not absorb water). In some instances, these variables are controlled by the selection of materials used in the manufacturing process.

In some embodiments, the controlled release matrix is configured to release at least about 40% and up to about 60%, or at least 50% of the bioactive agent within 24 hours of administration. In another embodiment, the controlled release matrix is configured to release at least about 80% or up to about 100%, or at least 90% of the bioactive agent within 7 days after administration.

In some embodiments, the controlled release matrix is biodegradable. In some embodiments, the controlled release matrix includes a biodegradable polyester. Examples of biodegradable polyesters include, but are not limited to: polycaprolactone (PCL), polylactic acid (PLA), polyglycolide (PGA), and copolymers thereof, such as poly(lactic-co-glycolic acid) polymers (PLGA) and poly(glycolide-co-caprolactone) (PGC). PCL refers to a biodegradable polyester prepared by ring opening polymerization of ε-caprolactone using a catalyst such as stannous octanoate. PCL has a melting point of about 60° C. and is degraded by hydrolysis of its ester linkages under physiological conditions. PLA is a biodegradable, thermoplastic polyester that can be produced by bacterial fermentation of renewable resources such as corn, starch or sugarcane and has a melting temperature between about 173° C. and about 178° C. PGA is a biodegradable, thermoplastic polyester prepared from glycolic acid by polycondensation or ring-opening polymerization. It has a melting point of between about 225° C. to about 230° C. A PLGA polymer refers to a biodegradable copolymer of lactic and glycolic acid formed by random ring-opening co-polymerization of monomers of glycolic acid and lactic acid. During polymerization, the monomeric units are linked together by ester linkages, thus yielding an aliphatic polyester. PLGAs are amorphous and have a glass transition temperature between about 40° C. and 60° C. In general, the PLGA copolymer has a weight average molecular weight between about 1000 Da to about 50,000 Da, or between about 5000 Da and 25,000 Da. The ratio of lactic acid to glycolic acid might vary. In general, and increase in the amount of lactic acid results in a polymer that degrades more slowly. An increase in glycolic acid results in a polymer that degrades more quickly. Additionally, an increase in glycolic acid tends to decrease the Tg and water penetration into the polymer, which can result in a faster release of compounds. In general, the ratio of lactic acid to glycolic acid is between about 100:0 to about 25:75, or between about 60:40 and 40:60, or about 50:50. Other suitable biodegradable polymers include, but are not limited to, poly(trimethylene carbonate) (PTMC), polydioxanone (PDO), poly(4-hydroxy butyrate) (PHB), and poly(butylene succinate) (PBS), poly(trimethylene carbonate) (PTMC), polydioxanone (PDO), poly(4-hydroxy butyrate) (PHB), and poly(butylene succinate) (PBS). In some embodiments, the polymeric material or polymer is biostable. Examples of biostable polymers include, but are not limited to polyurethanes, silicone rubbers, styrene-isobutylene-styrene block copolymers, ether-ester block copolymers (e.g. RTP 1500-40D from RTP Co.) and vinyl materials, including but not limited to poly(ethylene-co-vinyl acetate) (PEVA). In some embodiments, the controlled release matrix includes an elastomeric polymeric material that includes a copolymer with an elastomeric (or “soft”) component and a non-elastomeric (or “hard”) component. In another embodiment, the elastomeric polymeric material includes a polymeric blend having an elastomeric component and a non-elastomeric component. In some embodiments, the compliant polymer or polymeric material is thermoplastic. As used herein, the term “thermoplastic” refers to a polymer or polymeric material that can be softened by heat, hardened by cooling and then softened by heat over and over again. In general, thermoplastic materials are not cross-linked. However, in another embodiment, the compliant polymer or polymeric material might be cross-linked.

The bioactive agent is incorporated into the controlled release matrix, if used, using any suitable technique, such as any of various techniques known to the skilled artisan. In one embodiment, the bioactive agent is dispersed throughout the controlled release matrix. Techniques for preparing the controlled release matrix include, but are not limited to, melt extrusion processes, injection molding, or spray casting. In a typical melt extrusion process, a mixture that includes the polymeric material and bioactive agent is combined in an extruder, heated to a temperature at which the polymeric material melts and then discharged through an orifice of the desired cross-sectional shape. The extruded material is collected under controlled conditions (e.g. speed, temperature and humidity) to obtain a product with the desired dimensions. In one embodiment, the mass flow rate of the extrudate and the collection speed of the final extruded form might be controlled to achieve the desired physical dimensions. For example, if the final extruded form is a film, then the collection speed of the film might be increased relative to the mass flow rate of the extrudate to decrease the film thickness, and conversely to increase the film thickness. The extrudate is discharged through an orifice in the molten state, allowing elongation of the extrudate to its final dimension. The extrudate is subsequently cooled by exposure to ambient conditions, a chilled liquid or gas bath, or exposure to a temperature controlled surface such as a cooled roller in order to solidify the extrudate. In one embodiment, the melt extrusion process is used to form a film. In an alternate embodiment, the melt extrusion process is used to form pellets or beads that might be subsequently molded into the desired film or collar configuration. Some of the advantages of melt extrusion processes include: the absence of organic solvents and high throughput, continuous manufacturing. In general, the processing temperature is sufficient to melt the polymeric material without adversely affecting the biological activity of the bioactive agent. In general, the processing temperature is at least about 80° C. or about 100° C., and less than about 180° C., less than 160° C., or between about 110° C. and about 150° C. In some embodiments, the specific temperature is dependent on the melting and degradation temperatures of the polymeric materials and bioactive agent. Furthermore, melt-processing provides the ability for continuous operation, the ability to control operating parameters, and the ability to scale up manufacturing. In an alternate embodiment, an injection molding process is used. In a typical injection molding process, a mixture that includes the polymeric material and bioactive agent is fed into a vessel where it heated to a temperature sufficient to melt the polymeric material and then forced into a mold cavity where it cools and hardens to the configuration of the mold cavity. The conditions (e.g. temperature and pressure) will depend upon the material being molded. In one embodiment, the injection molding process is used to form a film or a collar. In yet another embodiment, a solvent casting technique is used. In a typical solvent casting process, the polymeric material and bioactive agent are combined with a suitable solvent to form a polymeric solution which is then cast on a substrate. The solvent is then removed to form a film, for example, by evaporation. In one embodiment, the solvent is removed under a vacuum (e.g. between about 15 in Hg and about 28 in Hg, depending upon the volatility of the solvent). In another embodiment, the solvent is removed at an elevated temperature (e.g. between about 30° C. and about 80° C.). In an alternate embodiment, the polymeric solution is applied to the substrate by a spray coating process. In a spray coating process, the polymeric solution is fed to the spray nozzle, for example and ultrasonic spray nozzle, at a controlled rate by a positive displacement pump. The spray nozzle and substrate are moved in relative motion to each other at controlled speed to achieve the desired coating thickness. The spray nozzle is mounted on a three-axis motion control system (x-y-z) which is capable of controlling the speed and position of the spray head relative to the substrate. In addition, if the substrate is a rolled film, it is traversed below the spray head by a roll to roll unwinding and winding apparatus. The coating width is controlled by moving the spray nozzle in a specified path across the width of the substrate. In addition, the height (z) of the spray nozzle above the substrate might be increased to achieve a wider coating width. In some instances, solvent forms a true solution with the components therein. In certain instances, the bioactive agent is soluble in the solvent or form a dispersion within the solvent. Any suitable solvents is optionally used, such as, by way of non-limiting example, alcohols (e.g. methanol, butanol, propanol and isopropanol), alkanes (e.g. halogenated or unhalogenated alkanes such as hexane, cyclohexane, methylene chloride and chloroform), amides (e.g. dimethylformamide), ethers (e.g. tetrahydrofuran (THF), dioxolane, and dioxane), ketones (e.g. methyl ethyl ketone, acetone), aromatic compounds (e.g. toluene and xylene), nitriles (e.g. acetonitrile) and esters (e.g. ethyl acetate). THF and chloroform have been found to be suitable solvents due to their excellent solvency for a variety of polymers and bioactive agents.

In certain embodiments, one or more compositions or formulations of a 5-HT receptor agonist or a pharmaceutically acceptable salt, solvate, metabolite, derivative, or prodrug thereof described herein further comprise an excipient. In some embodiments, aqueous suspensions of the pharmaceutical composition disclosed herein contain pharmaceutically acceptable excipients, such as a suspending agent (e.g. methyl cellulose), a wetting agent (e.g. lecithin, lysolecithin and/or a long-chain fatty alcohol), as well as coloring agents, preservatives, flavoring agents, and the like.

Pharmaceutical preparations for oral use are obtained using any suitable process, such as by combining active with a solid excipient, optionally grinding a resulting mixture, and processing the mixture of granules, after adding suitable auxiliaries, if desired, to obtain tablets or dragee cores. Suitable excipients are, in some instances, fillers such as sugars, including lactose, sucrose, mannitol, or sorbitol; flavoring elements, cellulose preparations such as, for example, maize starch, wheat starch, rice starch, potato starch, gelatin, gum tragacanth, methyl cellulose, hydroxypropylmethyl-cellulose, sodium carboxymethylcellulose, and/or PVP. If desired, disintegrating agents might be added, such as the cross-linked PVP, agar, or alginic acid or a salt thereof such as sodium alginate. The active compounds might also be formulated as a sustained release preparation.

Pharmaceutical preparations that are optionally used orally include push-fit capsules made of gelatin, as well as soft, sealed capsules made of gelatin and a plasticizer, such as glycerol or sorbitol. The push-fit capsules might contain the active ingredients in admixture with filler such as lactose, binders such as starches, and/or lubricants such as talc or magnesium stearate and, optionally, stabilizers. In soft capsules, the active compounds might be dissolved or suspended in suitable liquids, such as fatty oils, liquid paraffin, or liquid polyethylene glycols. In addition, stabilizers might be added. All formulations for oral administration should be in dosages suitable for administration.

For injection, the pharmaceutical compositions disclosed herein are optionally formulated in aqueous solutions, preferably in physiologically compatible buffers such as Hank's solution, Ringer's solution, or physiological saline buffer. Such compositions might also include one or more excipients, for example, preservatives, solubilizers, fillers, lubricants, stabilizers, albumin, and the like. Methods of formulation are known in the art, for example, as disclosed in Remington's Pharmaceutical Sciences, latest edition, Mack Publishing Co., Easton, Pa. These pharmaceutical compositions might also be formulated for transmucosal administration, buccal administration, for administration by inhalation, for parental administration, for transdermal administration, and rectal administration.

In addition to the disclosed formulations, the pharmaceutical compositions are optionally formulated as a depot preparation. Such long acting formulations might be administered by implantation or transcutaneous delivery (for example subcutaneously or intramuscularly), intramuscular injection or use of a transdermal patch. Thus, for example, the pharmaceutical compositions are optionally formulated with suitable polymeric or hydrophobic materials (for example as an emulsion in an acceptable oil) or ion exchange resins, or as sparingly soluble derivatives, for example, as a sparingly soluble salt.

In some embodiments, the pharmaceutical formulations include, but are not limited to, aqueous liquid dispersions, self-emulsifying dispersions, solid solutions, liposomal dispersions, aerosols, solid dosage forms, powders, immediate-release formulations, controlled release formulations, fast melt formulations, tablets, capsules, pills, delayed release formulations, extended release formulations, pulsatile release formulations, multiparticulate formulations (e.g. nanoparticle formulations), and mixed immediate and controlled release formulations.

In some instances, the pharmaceutical formulation includes multiparticulate formulations. In some instances, the pharmaceutical formulation includes nanoparticle formulations. In some instances, nanoparticles comprise cyclodextrins or lipids. In some cases, nanoparticles comprise solid lipid nanoparticles, polymeric nanoparticles, self-emulsifying nanoparticles, liposomes, microemulsions, or micellar solutions.

In some instances, a nanoparticle includes a core or a core and a shell, as in a core-shell nanoparticle.

In some instances, a nanoparticle is further coated with molecules for attachment of functional elements. In some instances, a coating comprises chondroitin sulfate, dextran sulfate, carboxymethyl dextran, alginic acid, pectin, carragheenan, fucoidan, agaropectin, porphyran, karaya gum, gellan gum, xanthan gum, hyaluronic acids, glucosamine, galactosamine, chitin (or chitosan), polyglutamic acid, polyaspartic acid, lysozyme, cytochrome C, ribonuclease, trypsinogen, chymotrypsinogen, α-chymotrypsin, polylysine, polyarginine, histone, protamine, ovalbumin or dextrin or cyclodextrin.

In some cases, a nanoparticle has at least one dimension of less than about 500 nm, 400 nm, 300 nm, 200 nm, or 100 nm.

In some embodiments, the pharmaceutical formulations include a carrier or carrier materials selected on the basis of compatibility with the composition disclosed herein, and the release profile properties of the desired dosage form. Pharmaceutically compatible carrier materials include, but are not limited to, acacia, gelatin, colloidal silicon dioxide, calcium glycerophosphate, calcium lactate, maltodextrin, glycerin, magnesium silicate, PVP, cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and any combination thereof. See, e.g. Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

In some instances, the pharmaceutical formulations further include pH adjusting agents or buffering agents which include acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some instances, the pharmaceutical formulations include binder which are used to hold a 5HT receptor agonist or a pharmaceutically acceptable salt, solvate, metabolite, derivative, or prodrug thereof and inactive ingredients together in a cohesive mix. Suitable binders include, but are not limited to, carboxymethylcellulose, methylcellulose (e.g. Methocel®), hydroxypropylmethylcellulose (e.g. Hypromellose USP Pharmacoat-603, hydroxypropylmethylcellulose acetate stearate (Aqoate HS-LF and HS), hydroxyethylcellulose, hydroxypropylcellulose (e.g. Klucel®), ethylcellulose (e.g. Ethocel®), and microcrystalline cellulose (e.g. Avicel®), microcrystalline dextrose, amylose, magnesium aluminum silicate, polysaccharide acids, bentonites, gelatin, PVP/vinyl acetate copolymer, crospovidone, povidone, starch, pregelatinized starch, tragacanth, dextrin, a sugar, such as sucrose (e.g. Dipac®), glucose, dextrose, molasses, mannitol, sorbitol, xylitol (e.g. Xylitab®), lactose, a natural or synthetic gum such as acacia, tragacanth, ghatti gum, mucilage of isapol husks, starch, PVP (e.g. Povidone® CL, Kollidon® CL, Polyplasdone® XL-10, and Povidone® K-12), larch arabogalactan, Veegum®, polyethylene glycol, waxes, sodium alginate, and any combination thereof.

In some instances, the pharmaceutical formulations further include diluent which are used to stabilize a 5HT receptor agonist or a pharmaceutically acceptable salt, solvate, metabolite, derivative, or prodrug thereof because they provide a more stable environment. Salts dissolved in buffered solutions (which also provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds include e.g. lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and any combination thereof.

In some cases, the pharmaceutical formulations include disintegration agents or disintegrants to facilitate the breakup or disintegration of a substance. The term “disintegrate” includes both the dissolution and dispersion of the dosage form when contacted with gastrointestinal fluid. Examples of disintegration agents include a starch, e.g. a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g. Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked PVP, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and any combination thereof.

In some instances, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and any combination thereof.

Lubricants and glidants are also optionally included in the pharmaceutical formulations disclosed herein for preventing, reducing or inhibiting adhesion or friction of materials. Exemplary lubricants include, e.g. stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g. PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and any combination thereof.

Plasticizers include compounds used to soften the microencapsulation material or film coatings to make them less brittle. Suitable plasticizers include, e.g. PEGs such as PEG 300, PEG 400, PEG 600, PEG 1450, PEG 3350, and PEG 800, stearic acid, propylene glycol, oleic acid, triethyl cellulose and triacetin. Plasticizers also function as dispersing agents or wetting agents.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, PVP, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and any combination thereof.

Stabilizers include compounds such as any antioxidation agents, buffers, acids, preservatives and any combination thereof.

Suspending agents include compounds such as PVP, e.g. PVP K12, PVP K17, PVP K25, or PVP K30, vinyl pyrrolidone/vinyl acetate copolymer (S630), PEG, e.g. the PEG has a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, Polysorbate 80, hydroxyethylcellulose, sodium alginate, gums, such as, e.g. gum tragacanth and gum acacia, guar gum, xanthans, including xanthan gum, sugars, cellulosics, such as, e.g. sodium carboxymethylcellulose, methylcellulose, sodium carboxymethylcellulose, hydroxypropylmethylcellulose, hydroxyethylcellulose, Polysorbate 80, sodium alginate, polyethoxylated sorbitan monolaurate, polyethoxylated sorbitan monolaurate, povidone, and any combination thereof.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g. Pluronic® (BASF), and any combination thereof. Additional surfactants include polyoxyethylene fatty acid glycerides and vegetable oils, e.g. polyoxyethylene (60) hydrogenated castor oil; and polyoxyethylene alkylethers and alkylphenyl ethers, e.g. octoxynol 10, octoxynol 40. Sometimes, surfactants are included to enhance physical stability or for other purposes.

Viscosity enhancing agents include, e.g. methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts, and any combination thereof.

Antifoaming agents are chemical additive that reduces and hinders the formation of foam in the preparation of an oral liquid formulation. The terms antifoaming agent and defoamer are often used interchangeably. Commonly used agents are insoluble oils, polydimethylsiloxanes (e.g. simethicone) and other silicones, certain alcohols, stearates and glycols. The additive is used to prevent formation of foam or is added to break foam already formed. Antifoaming agents reduce foaming in the preparation of an oral liquid formulation which might result in coagulation of aqueous dispersions. In some embodiments, the 5HT receptor agonist compositions described herein comprise an antifoaming agent. In some embodiments, the antifoaming agent is simethicone.

In some embodiments, there is a considerable overlap between excipients used in the pharmaceutical compositions, formulations, and dosage forms of a 5HT receptor agonist or a pharmaceutically acceptable salt, solvate, metabolite, derivative, or prodrug thereof described herein. Thus, the above-listed additives should be taken as merely exemplary, and not limiting, of the types of additives that might be included in solid dosage forms of the pharmaceutical compositions described herein.

### Methods of Pharmaceutical Formulations and Routes of Administration

In some embodiments, 5HT receptor agonists or pharmaceutical compositions or formulations described herein are administered to a subject by multiple administration routes, including but not limited to, oral, parenteral (e.g. intravenous, subcutaneous, intramuscular), intranasal, inhalation, buccal, topical, rectal, or transdermal administration routes. In some embodiments, pharmaceutical compositions described herein, which include 5HT receptor agonist, or pharmaceutically acceptable salt, solvate, metabolite, derivative, or prodrug thereof, are formulated into any suitable dosage form, including but not limited to, emulsions suitable for injection, nanosuspensions suitable for injection, aqueous oral dispersions, liquids, gels, syrups, elixirs, slurries, suspensions, aerosols, controlled release formulations, fast melt formulations, effervescent formulations, lyophilized formulations, tablets, powders, pills, dragees, capsules, delayed release formulations, extended release formulations, pulsatile release formulations, multiparticulates formulations, and mixed immediate-release and controlled release formulations.

In some embodiments, the pharmaceutical composition for oral use is a tablet, (including a suspension tablet, a fast melt tablet, a bite-disintegration tablet, a rapid-disintegration tablet, an effervescent tablet, or a caplet), a pill, a powder (including a sterile packaged powder, a dispensable powder, or an effervescent powder), a capsule (including both soft or hard capsules, e.g. capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In some embodiments, the pharmaceutical composition for oral use is a solid dosage form, e.g. tablets, effervescent tablets, and capsules. In some embodiments, the solid dosage forms are prepared by mixing particles of 5HT receptor agonist, or pharmaceutically acceptable salt, solvate, metabolite, derivative, or prodrug thereof, with one or more pharmaceutical excipients to form a bulk blend composition. When referring to these bulk blend compositions as homogeneous, it is meant that the particles of 5HT receptor agonist, or pharmaceutically acceptable salt, solvate, metabolite, derivative, or prodrug thereof, are dispersed evenly throughout the composition so that the composition might be subdivided into equally effective unit dosage forms, such as tablets, pills, and capsules. The individual unit dosages might also include film coatings, which disintegrate upon oral ingestion or upon contact with diluent.

For oral administration, the pharmaceutical compositions disclosed herein are, in some instances, formulated readily by combining the active compound(s) with pharmaceutically acceptable carriers well known in the art. Such carriers enable the compositions disclosed herein to be formulated as tablets, including chewable tablets, pills, dragees, capsules, lozenges, hard candy, liquids, gels, syrups, slurries, powders, suspensions, elixirs, wafers, and the like, for oral ingestion by a patient to be treated. Such formulations might comprise pharmaceutically acceptable carriers including solid diluents or fillers, sterile aqueous media and various non-toxic organic solvents. Generally, the compositions disclosed herein will be included at concentration levels ranging from about 0.5%, about 5%, about 10%, about 20%, or about 30% to about 50%, about 60%, about 70%, about 80% or about 90% by weight of the total composition of oral dosage forms, in an amount sufficient to provide a desired unit of dosage.

### Dosage

In some embodiments, the amount of 5HT receptor agonist, or pharmaceutically acceptable salt, solvate, metabolite, derivative, or prodrug thereof (e.g., used in a pharmaceutical composition described herein) is below a threshold that produces an adverse event (e.g., a clinically important effect (e.g., clinically important impairment of the individual), altered (e.g., visual, auditory, body, time and space) perception, altered cognition, impaired attention, drowsiness, confusion, or the like in the individual).

In some embodiments, the amount of 5HT receptor agonist, or pharmaceutically acceptable salt, solvate, metabolite, derivative, or prodrug thereof used in a pharmaceutical composition is about 1 mg/ml to about 30 mg/ml. In some embodiments, the amount of 5HT receptor agonist, or pharmaceutically acceptable salt, solvate, metabolite, derivative, or prodrug thereof used in a pharmaceutical composition is about 0.1 mg/ml to about 10 mg/ml. In some embodiments, the amount of 5HT receptor agonist, or pharmaceutically acceptable salt, solvate, metabolite, derivative, or prodrug thereof used in a pharmaceutical composition is about 0.1 mg/ml, about 0.2 mg/ml, about 0.3 mg/ml, about 0.4 mg/ml, about 0.5 mg/ml, about 0.6 mg/ml, about 0.7 mg/ml, about 0.8 mg/ml, about 0.9 mg/ml, about 1 mg/ml, about 1.1 mg/ml, about 1.2 mg/ml, about 1.3 mg/ml, about 1.4 mg/ml, about 1.5 mg/ml, about 1.6 mg/ml, about 1.7 mg/ml, about 1.8 mg/ml, about 1.9 mg/ml, about 2 mg/ml, about 2.1 mg/ml, about 2.2 mg/ml, about 2.3 mg/ml, about 2.4 mg/ml, about 2.5 mg/ml, about 2.6 mg/ml, about 2.7 mg/ml, about 2.8 mg/ml, about 2.9 mg/ml, about 3 mg/ml, about 3.1 mg/ml, about 3.2 mg/ml, about 3.3 mg/ml, about 3.4 mg/ml, about 3.5 mg/ml, about 3.6 mg/ml, about 3.7 mg/ml, about 3.8 mg/ml, about 3.9 mg/ml, about 4 mg/ml, about 4.1 mg/ml, about 4.2 mg/ml, about 4.3 mg/ml, about 4.4 mg/ml, about 4.5 mg/ml, about 4.6 mg/ml, about 4.7 mg/ml, about 4.8 mg/ml, about 4.9 mg/ml, about 5 mg/ml, about 5.1 mg/ml, about 5.2 mg/ml, about 5.3 mg/ml, about 5.4 mg/ml, about 5.5 mg/ml, about 5.6 mg/ml, about 5.7 mg/ml, about 5.8 mg/ml, about 5.9 mg/ml, about 6 mg/ml, about 6.1 mg/ml, about 6.2 mg/ml, about 6.3 mg/ml, about 6.4 mg/ml, about 6.5 mg/ml, about 6.6 mg/ml, about 6.7 mg/ml, about 6.8 mg/ml, about 6.9 mg/ml, about 7 mg/ml, about 7.1 mg/ml, about 7.2 mg/ml, about 7.3 mg/ml, about 7.4 mg/ml, about 7.5 mg/ml, about 7.6 mg/ml, about 7.7 mg/ml, about 7.8 mg/ml, about 7.9 mg/ml, about 8 mg/ml, about 8.1 mg/ml, about 8.2 mg/ml, about 8.3 mg/ml, about 8.4 mg/ml, about 8.5 mg/ml, about 8.6 mg/ml, about 8.7 mg/ml, about 8.8 mg/ml, about 8.9 mg/ml, about 9 mg/ml, about 9.1 mg/ml, about 9.2 mg/ml, about 9.3 mg/ml, about 9.4 mg/ml, about 9.5 mg/ml, about 9.6 mg/ml, about 9.7 mg/ml, about 9.8 mg/ml, about 9.9 mg/ml, about 10 mg/ml, about 10.1 mg/ml, about 10.2 mg/ml, about 10.3 mg/ml, about 10.4 mg/ml, about 10.5 mg/ml, about 10.6 mg/ml, about 10.7 mg/ml, about 10.8 mg/ml, about 10.9 mg/ml, about 11 mg/ml, about 11.1 mg/ml, about 11.2 mg/ml, about 11.3 mg/ml, about 11.4 mg/ml, about 11.5 mg/ml, about 11.6 mg/ml, about 11.7 mg/ml, about 11.8 mg/ml, about 11.9 mg/ml, about 12 mg/ml, about 12.1 mg/ml, about 12.2 mg/ml, about 12.3 mg/ml, about 12.4 mg/ml, about 12.5 mg/ml, about 12.6 mg/ml, about 12.7 mg/ml, about 12.8 mg/ml, about 12.9 mg/ml, about 13 mg/ml, about 13.1 mg/ml, about 13.2 mg/ml, about 13.3 mg/ml, about 13.4 mg/ml, about 13.5 mg/ml, about 13.6 mg/ml, about 13.7 mg/ml, about 13.8 mg/ml, about 13.9 mg/ml, about 14 mg/ml, about 14.1 mg/ml, about 14.2 mg/ml, about 14.3 mg/ml, about 14.4 mg/ml, about 14.5 mg/ml, about 14.6 mg/ml, about 14.7 mg/ml, about 14.8 mg/ml, about 14.9 mg/ml, about 15 mg/ml, about 15.5 mg/ml, about 16 mg/ml, about 16.5 mg/ml, about 17 mg/ml, about 17.5 mg/ml, about 18 mg/ml, about 18.5 mg/ml, about 19 mg/ml, about 19.5 mg/ml, about 20 mg/ml, about 21 mg/ml, about 22 mg/ml, about 23 mg/ml, about 24 mg/ml, about 25 mg/ml, about 27.5 mg/ml, about 30 mg/ml.

In some embodiments, the amount of a 5HT receptor agonist or a pharmaceutically acceptable salt, solvate, metabolite, derivative, or prodrug thereof in the pharmaceutical composition corresponds to about 0.8 mg/ml to about 24 mg/mi of the 5HT receptor agonist or a pharmaceutically acceptable salt, solvate, metabolite, derivative, or prodrug thereof. In other embodiments, the amount of 5HT receptor agonist, or pharmaceutically acceptable salt, solvate, metabolite, derivative, or prodrug thereof in the pharmaceutical composition corresponds to about 0.8 mg/ml, about 0.9 mg/ml, about 1 mg/ml, about 1.1 mg/ml, about 1.2 mg/ml, about 1.3 mg/ml, about 1.4 mg/ml, about 1.5 mg/ml, about 1.6 mg/ml, about 1.7 mg/ml, about 1.8 mg/ml, about 1.9 mg/ml, about 2 mg/ml, about 2.1 mg/ml, about 2.2 mg/ml, about 2.3 mg/ml, about 2.4 mg/ml, about 2.5 mg/ml, about 2.6 mg/ml, about 2.7 mg/ml, about 2.8 mg/ml, about 2.9 mg/ml, about 3 mg/ml, about 3.1 mg/ml, about 3.2 mg/ml, about 3.3 mg/ml, about 3.4 mg/ml, about 3.5 mg/ml, about 3.6 mg/ml, about 3.7 mg/ml, about 3.8 mg/ml, about 3.9 mg/ml, about 4 mg/ml, about 4.1 mg/ml, about 4.2 mg/ml, about 4.3 mg/ml, about 4.4 mg/ml, about 4.5 mg/ml, about 4.6 mg/ml, about 4.7 mg/ml, about 4.8 mg/ml, about 4.9 mg/ml, about 5 mg/ml, about 5.1 mg/ml, about 5.2 mg/ml, about 5.3 mg/ml, about 5.4 mg/ml, about 5.5 mg/ml, about 5.6 mg/ml, about 5.7 mg/ml, about 5.8 mg/ml, about 5.9 mg/ml, about 6 mg/ml, about 6.1 mg/ml, about 6.2 mg/ml, about 6.3 mg/ml, about 6.4 mg/ml, about 6.5 mg/ml, about 6.6 mg/ml, about 6.7 mg/ml, about 6.8 mg/ml, about 6.9 mg/ml, about 7 mg/ml, about 7.1 mg/ml, about 7.2 mg/ml, about 7.3 mg/ml, about 7.4 mg/ml, about 7.5 mg/ml, about 7.6 mg/ml, about 7.7 mg/ml, about 7.8 mg/ml, about 7.9 mg/ml, about 8 mg/ml, about 8.1 mg/ml, about 8.2 mg/ml, about 8.3 mg/ml, about 8.4 mg/ml, about 8.5 mg/ml, about 8.6 mg/ml, about 8.7 mg/ml, about 8.8 mg/ml, about 8.9 mg/ml, about 9 mg/ml, about 9.1 mg/ml, about 9.2 mg/ml, about 9.3 mg/ml, about 9.4 mg/ml, about 9.5 mg/ml, about 9.6 mg/ml, about 9.7 mg/ml, about 9.8 mg/ml, about 9.9 mg/ml, about 10 mg/ml, about 10.1 mg/ml, about 10.2 mg/ml, about 10.3 mg/ml, about 10.4 mg/ml, about 10.5 mg/ml, about 10.6 mg/ml, about 10.7 mg/ml, about 10.8 mg/ml, about 10.9 mg/ml, about 11 mg/ml, about 11.1 mg/ml, about 11.2 mg/ml, about 11.3 mg/ml, about 11.4 mg/ml, about 11.5 mg/ml, about 11.6 mg/ml, about 11.7 mg/ml, about 11.8 mg/ml, about 11.9 mg/ml, about 12 mg/ml, about 12.1 mg/ml, about 12.2 mg/ml, about 12.3 mg/ml, about 12.4 mg/ml, about 12.5 mg/ml, about 12.6 mg/ml, about 12.7 mg/ml, about 12.8 mg/ml, about 12.9 mg/ml, about 13 mg/ml, about 13.1 mg/ml, about 13.2 mg/ml, about 13.3 mg/ml, about 13.4 mg/ml, about 13.5 mg/ml, about 13.6 mg/ml, about 13.7 mg/ml, about 13.8 mg/ml, about 13.9 mg/ml, about 14 mg/ml, about 14.1 mg/ml, about 14.2 mg/ml, about 14.3 mg/ml, about 14.4 mg/ml, about 14.5 mg/ml, about 14.6 mg/ml, about 14.7 mg/ml, about 14.8 mg/ml, about 14.9 mg/ml, about 15 mg/ml, about 15.5 mg/ml, about 16 mg/ml, about 16.5 mg/ml, about 17 mg/ml, about 17.5 mg/ml, about 18 mg/ml, about 18.5 mg/ml, about 19 mg/ml, about 19.5 mg/ml, about 20 mg/ml, about 21 mg/ml, about 22 mg/ml, about 23 mg/ml, or about 24 mg/ml of a 5HT receptor agonist or a pharmaceutically acceptable salt, solvate, metabolite, derivative, or prodrug thereof.

In some embodiments, the amount of 5HT receptor agonist, or pharmaceutically acceptable salt, solvate, metabolite, derivative, or prodrug thereof used in a pharmaceutical composition is at least 0.001 mg/kg or more.

In some embodiments, the amount of 5HT receptor agonist, or pharmaceutically acceptable salt, solvate, metabolite, derivative, or prodrug thereof used in a pharmaceutical composition is at most 60 mg/kg or less.

In some embodiments, the amount of 5HT receptor agonist, or pharmaceutically acceptable salt, solvate, metabolite, derivative, or prodrug thereof used in a pharmaceutical composition is about 0.001 mg/kg to about 60 mg/kg (e.g., about 0.01 mg/kg to about 60 mg/kg, about 0.1 mg/kg to about 60 mg/kg, or about 1 mg/kg to about 60 mg/kg). In some embodiments, the amount of 5HT receptor agonist, or pharmaceutically acceptable salt, solvate, metabolite, derivative, or prodrug thereof used in a pharmaceutical composition is about 1 mg/kg to about 60 mg/kg (e.g., about 1 mg/kg to about 50 mg/kg, about 1 mg/kg to about 40 mg/kg, about 1 mg/kg to about 30 mg/kg, about 1 mg/kg to about 20 mg/kg, or about 1 mg/kg to about 10 mg/kg). In some embodiments, the amount of 5HT receptor agonist, or pharmaceutically acceptable salt, solvate, metabolite, derivative, or prodrug thereof used in a pharmaceutical composition is about 1 mg/kg to about 10 mg/kg.

In some embodiments, the amount of 5HT receptor agonist, or pharmaceutically acceptable salt, solvate, metabolite, derivative, or prodrug thereof used in a pharmaceutical composition is at least 0.001 mg or more.

In some embodiments, the amount of 5HT receptor agonist, or pharmaceutically acceptable salt, solvate, metabolite, derivative, or prodrug thereof used in a pharmaceutical composition is at most 60 mg or less.

In some embodiments, the pharmaceutical composition comprises the 5HT receptor agonist or a pharmaceutically acceptable salt, solvate, metabolite, derivative, or prodrug thereof in an amount of about 0.0001 mg to about 600 mg (e.g. about 0.001 mg to about 100 mg, about 0.2 mg to about 25 mg, about 10 mg to about 50 mg, or the like). In some embodiments, the amount of 5HT receptor agonist, or pharmaceutically acceptable salt, solvate, metabolite, derivative, or prodrug thereof used in a pharmaceutical composition is about 0.001 mg to about 60 mg (e.g., about 0.01 mg to about 60 mg, about 0.1 mg to about 60 mg, or about 1 mg to about 60 mg). In some embodiments, the amount of 5HT receptor agonist, or pharmaceutically acceptable salt, solvate, metabolite, derivative, or prodrug thereof used in a pharmaceutical composition is about 1 mg to about 60 mg (e.g., about 1 mg to about 50 mg, about 1 mg to about 40 mg, about 1 mg to about 30 mg, about 1 mg to about 20 mg, or about 1 mg to about 10 mg). In some embodiments, the amount of 5HT receptor agonist, or pharmaceutically acceptable salt, solvate, metabolite, derivative, or prodrug thereof used in a pharmaceutical composition is about 1 mg to about 10 mg. In some embodiments, the pharmaceutical composition comprises the 5HT receptor agonist or a pharmaceutically acceptable salt, solvate, metabolite, derivative, or prodrug thereof in an amount of about 0.1 mg to about 50 mg (e.g. about 0.1 mg to about 10 mg, about 0.2 mg to about 5 mg, about 10 mg to about 50 mg, or the like). In some embodiments, the pharmaceutical composition comprises the 5HT receptor agonist or a pharmaceutically acceptable salt, solvate, metabolite, derivative, or prodrug thereof in an amount of about 0.5 mg to about 5 mg. In some embodiments, the pharmaceutical composition comprises the 5HT receptor agonist or a pharmaceutically acceptable salt, solvate, metabolite, derivative, or prodrug thereof in an amount of about 0.5, 1, 1.5, 2, 2.5, 3, 3.5, 4, 4.5, or 5 mg. In some embodiments, the pharmaceutical composition comprises the 5HT receptor agonist or a pharmaceutically acceptable salt, solvate, metabolite, derivative, or prodrug thereof in an amount of about 1, 2, 3, 4, 5, 6, 7, 8, 9, 10, 11, 12, 13, 14, 15 mg. In some embodiments, the pharmaceutical composition comprises the 5HT receptor agonist or a pharmaceutically acceptable salt, solvate, metabolite, derivative, or prodrug thereof in an amount of about 1 mg to about 25, 2 mg to about 35 mg, about 5 mg to 30 mg, about 10 to 40 mg, about 25 mg to 35 mg. In some embodiments, the pharmaceutical composition comprises the 5HT receptor agonist or a pharmaceutically acceptable salt, solvate, metabolite, derivative, or prodrug thereof in an amount of about 16, 17, 18, 19, 20, 21, 22, 23, 24, 25, 26, 27, 28, 29, 30, 31, 32, 33, 34, 35, 36, 37, 38, 39, 40 mg. In some embodiments, the pharmaceutical composition is a low-dose pharmaceutical composition. In some embodiments, the pharmaceutical composition is an extended release composition.

In some embodiments, the amount of 5HT receptor agonist, or pharmaceutically acceptable salt, solvate, metabolite, derivative, or prodrug thereof used in a pharmaceutical composition is about 0.001 mg to about 20 mg. In some embodiments, the amount of 5HT receptor agonist, or pharmaceutically acceptable salt, solvate, metabolite, derivative, or prodrug thereof used in a pharmaceutical composition is about 0.005 mg to about 10 mg. In some embodiments, the amount of 5HT receptor agonist, or pharmaceutically acceptable salt, solvate, metabolite, derivative, or prodrug thereof used in a pharmaceutical composition is about 0.01 mg to about 5 mg. In some embodiments, the amount of 5HT receptor agonist, or pharmaceutically acceptable salt, solvate, metabolite, derivative, or prodrug thereof used in a pharmaceutical composition is about 0.05 mg to about 2.5 mg. In other embodiments, the amount of 5HT receptor agonist, or pharmaceutically acceptable salt, solvate, metabolite, derivative, or prodrug thereof in the pharmaceutical composition corresponds to about 0.001 mg, about 0.005 mg, about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.11 mg, about 0.12 mg, about 0.15 mg, about 0.17 mg, about 0.2 mg, about 0.23 mg, about 0.25 mg, about 0.28 mg, about 0.3 mg, about 0.33 mg, about 0.35 mg, about 0.37 mg, about 0.4 mg, about 0.43 mg, about 0.45 mg, about 0.47 mg, about 0.5 mg, about 0.53 mg, about 0.55 mg, about 0.57 mg, about 0.6 mg, about 0.63 mg, about 0.65 mg, about 0.67 mg, about 0.7 mg, about 0.73 mg, about 0.75 mg, about 0.78 mg, about 0.8 mg, about 0.83 mg, about 0.85 mg, about 0.87 mg, about 0.9 mg, about 0.95 mg, about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2 mg, about 2.1 mg, about 2.2 mg, about 2.3 mg, about 2.4 mg, about 2.5 mg, about 2.6 mg, about 2.7 mg, about 2.8 mg, about 2.9 mg, about 3 mg, about 3.1 mg, about 3.2 mg, about 3.3 mg, about 3.4 mg, about 3.5 mg, about 3.6 mg, about 3.7 mg, about 3.8 mg, about 3.9 mg, about 4 mg, about 4.1 mg, about 4.2 mg, about 4.3 mg, about 4.4 mg, about 4.5 mg, about 4.6 mg, about 4.7 mg, about 4.8 mg, about 4.9 mg, about 5 mg, about 6 mg, about 7 mg, about 8 mg, about 9 mg, about 10 mg, or about 11 mg.

In other embodiments, the amount of 5HT receptor agonist, or pharmaceutically acceptable salt, solvate, metabolite, derivative, or prodrug thereof in the pharmaceutical composition corresponds to no less than 0.001 mg, no less than 0.005 mg, no less than 0.01 mg, no less than 0.02 mg, no less than 0.03 mg, no less than 0.04 mg, no less than 0.05 mg, no less than 0.06 mg, no less than 0.07 mg, no less than 0.08 mg, no less than 0.09 mg, no less than 0.1 mg, no less than 0.11 mg, no less than 0.12 mg, no less than 0.15 mg, no less than 0.17 mg, no less than 0.2 mg, no less than 0.23 mg, no less than 0.25 mg, no less than 0.28 mg, no less than 0.3 mg, no less than 0.33 mg, no less than 0.35 mg, no less than 0.37 mg, no less than 0.4 mg, no less than 0.43 mg, no less than 0.45 mg, no less than 0.47 mg, no less than 0.5 mg, no less than 0.53 mg, no less than 0.55 mg, no less than 0.57 mg, no less than 0.6 mg, no less than 0.63 mg, no less than 0.65 mg, no less than 0.67 mg, no less than 0.7 mg, no less than 0.73 mg, no less than 0.75 mg, no less than 0.78 mg, no less than 0.8 mg, no less than 0.83 mg, no less than 0.85 mg, no less than 0.87 mg, no less than 0.9 mg, no less than 0.95 mg, no less than 1 mg, no less than 1.1 mg, no less than 1.2 mg, no less than 1.3 mg, no less than 1.4 mg, no less than 1.5 mg, no less than 1.6 mg, no less than 1.7 mg, no less than 1.8 mg, no less than 1.9 mg, no less than 2 mg, no less than 2.1 mg, no less than 2.2 mg, no less than 2.3 mg, no less than 2.4 mg, no less than 2.5 mg, no less than 2.6 mg, no less than 2.7 mg, no less than 2.8 mg, no less than 2.9 mg, no less than 3 mg, no less than 3.1 mg, no less than 3.2 mg, no less than 3.3 mg, no less than 3.4 mg, no less than 3.5 mg, no less than 3.6 mg, no less than 3.7 mg, no less than 3.8 mg, no less than 3.9 mg, no less than 4 mg, no less than 4.1 mg, no less than 4.2 mg, no less than 4.3 mg, no less than 4.4 mg, no less than 4.5 mg, no less than 4.6 mg, no less than 4.7 mg, no less than 4.8 mg, no less than 4.9 mg, no less than 5 mg, no less than 5.1 mg, no less than 5.2 mg, no less than 5.3 mg, no less than 5.4 mg, no less than 5.5 mg, no less than 5.6 mg, no less than 5.7 mg, no less than 5.8 mg, or no less than 5.9 mg.

In other embodiments, the amount of 5HT receptor agonist, or pharmaceutically acceptable salt, solvate, metabolite, derivative, or prodrug thereof in the pharmaceutical composition corresponds to no more than 0.005 mg, no more than 0.01 mg, no more than 0.02 mg, no more than 0.03 mg, no more than 0.04 mg, no more than 0.05 mg, no more than 0.06 mg, no more than 0.07 mg, no more than 0.08 mg, no more than 0.09 mg, no more than 0.1 mg, no more than 0.11 mg, no more than 0.12 mg, no more than 0.15 mg, no more than 0.17 mg, no more than 0.2 mg, no more than 0.23 mg, no more than 0.25 mg, no more than 0.28 mg, no more than 0.3 mg, no more than 0.33 mg, no more than 0.35 mg, no more than 0.37 mg, no more than 0.4 mg, no more than 0.43 mg, no more than 0.45 mg, no more than 0.47 mg, no more than 0.5 mg, no more than 0.53 mg, no more than 0.55 mg, no more than 0.57 mg, no more than 0.6 mg, no more than 0.63 mg, no more than 0.65 mg, no more than 0.67 mg, no more than 0.7 mg, no more than 0.73 mg, no more than 0.75 mg, no more than 0.78 mg, no more than 0.8 mg, no more than 0.83 mg, no more than 0.85 mg, no more than 0.87 mg, no more than 0.9 mg, no more than 0.95 mg, no more than 1 mg, no more than 1.1 mg, no more than 1.2 mg, no more than 1.3 mg, no more than 1.4 mg, no more than 1.5 mg, no more than 1.6 mg, no more than 1.7 mg, no more than 1.8 mg, no more than 1.9 mg, no more than 2 mg, no more than 2.1 mg, no more than 2.2 mg, no more than 2.3 mg, no more than 2.4 mg, no more than 2.5 mg, no more than 2.6 mg, no more than 2.7 mg, no more than 2.8 mg, no more than 2.9 mg, no more than 3 mg, no more than 3.1 mg, no more than 3.2 mg, no more than 3.3 mg, no more than 3.4 mg, no more than 3.5 mg, no more than 3.6 mg, no more than 3.7 mg, no more than 3.8 mg, no more than 3.9 mg, no more than 4 mg, no more than 4.1 mg, no more than 4.2 mg, no more than 4.3 mg, no more than 4.4 mg, no more than 4.5 mg, no more than 4.6 mg, no more than 4.7 mg, no more than 4.8 mg, no more than 4.9 mg, no more than 5 mg, no more than 5.1 mg, no more than 5.2 mg, no more than 5.3 mg, no more than 5.4 mg, no more than 5.5 mg, no more than 5.6 mg, no more than 5.7 mg, no more than 5.8 mg, no more than 5.9 mg, or no more than 6 mg.

In some embodiments, the amount of 5HT receptor agonist, or pharmaceutically acceptable salt, solvate, metabolite, derivative, or prodrug thereof used in a pharmaceutical composition is about 0.001 mg/kg to about 50 mg/kg. In some embodiments, the amount of 5HT receptor agonist, or pharmaceutically acceptable salt, solvate, metabolite, derivative, or prodrug thereof used in a pharmaceutical composition is about 0.005 mg/kg to about 10 mg/kg. In some embodiments, the amount of 5HT receptor agonist, or pharmaceutically acceptable salt, solvate, metabolite, derivative, or prodrug thereof used in a pharmaceutical composition is about 0.01 mg/kg to about 5 mg/kg. In some embodiments, the amount of 5HT receptor agonist, or pharmaceutically acceptable salt, solvate, metabolite, derivative, or prodrug thereof used in a pharmaceutical composition is about 0.05 mg/kg to about 1 mg/kg. In other embodiments, the amount of 5HT receptor agonist, or pharmaceutically acceptable salt, solvate, metabolite, derivative, or prodrug thereof in the pharmaceutical composition corresponds to about 0.001 mg/kg, about 0.005 mg/kg, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.11 mg/kg, about 0.12 mg/kg, about 0.15 mg/kg, about 0.17 mg/kg, about 0.2 mg/kg, about 0.23 mg/kg, about 0.25 mg/kg, about 0.28 mg/kg, about 0.3 mg/kg, about 0.33 mg/kg, about 0.35 mg/kg, about 0.37 mg/kg, about 0.4 mg/kg, about 0.43 mg/kg, about 0.45 mg/kg, about 0.47 mg/kg, about 0.5 mg/kg, about 0.53 mg/kg, about 0.55 mg/kg, about 0.57 mg/kg, about 0.6 mg/kg, about 0.63 mg/kg, about 0.65 mg/kg, about 0.67 mg/kg, about 0.7 mg/kg, about 0.73 mg/kg, about 0.75 mg/kg, about 0.78 mg/kg, about 0.8 mg/kg, about 0.83 mg/kg, about 0.85 mg/kg, about 0.87 mg/kg, about 0.9 mg/kg, about 0.95 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4 mg/kg, about 4.1 mg/kg, about 4.2 mg/kg, about 4.3 mg/kg, about 4.4 mg/kg, about 4.5 mg/kg, about 4.6 mg/kg, about 4.7 mg/kg, about 4.8 mg/kg, about 4.9 mg/kg, about 5 mg/kg, about 6 mg/kg, about 7 mg/kg, about 8 mg/kg, about 9 mg/kg, or about 10 mg/kg.

In other embodiments, the amount of 5HT receptor agonist, or pharmaceutically acceptable salt, solvate, metabolite, derivative, or prodrug thereof in the pharmaceutical composition corresponds to no less than 0.001 mg/kg, no less than 0.005 mg/kg, no less than 0.01 mg/kg, no less than 0.02 mg/kg, no less than 0.03 mg/kg, no less than 0.04 mg/kg, no less than 0.05 mg/kg, no less than 0.06 mg/kg, no less than 0.07 mg/kg, no less than 0.08 mg/kg, no less than 0.09 mg/kg, no less than 0.1 mg/kg, no less than 0.11 mg/kg, no less than 0.12 mg/kg, no less than 0.15 mg/kg, no less than 0.17 mg/kg, no less than 0.2 mg/kg, no less than 0.23 mg/kg, no less than 0.25 mg/kg, no less than 0.28 mg/kg, no less than 0.3 mg/kg, no less than 0.33 mg/kg, no less than 0.35 mg/kg, no less than 0.37 mg/kg, no less than 0.4 mg/kg, no less than 0.43 mg/kg, no less than 0.45 mg/kg, no less than 0.47 mg/kg, no less than 0.5 mg/kg, no less than 0.53 mg/kg, no less than 0.55 mg/kg, no less than 0.57 mg/kg, no less than 0.6 mg/kg, no less than 0.63 mg/kg, no less than 0.65 mg/kg, no less than 0.67 mg/kg, no less than 0.7 mg/kg, no less than 0.73 mg/kg, no less than 0.75 mg/kg, no less than 0.78 mg/kg, no less than 0.8 mg/kg, no less than 0.83 mg/kg, no less than 0.85 mg/kg, no less than 0.87 mg/kg, no less than 0.9 mg/kg, no less than 0.95 mg/kg, no less than 1 mg/kg, no less than 1.1 mg/kg, no less than 1.2 mg/kg, no less than 1.3 mg/kg, no less than 1.4 mg/kg, no less than 1.5 mg/kg, no less than 1.6 mg/kg, no less than 1.7 mg/kg, no less than 1.8 mg/kg, no less than 1.9 mg/kg, no less than 2 mg/kg, no less than 2.1 mg/kg, no less than 2.2 mg/kg, no less than 2.3 mg/kg, no less than 2.4 mg/kg, no less than 2.5 mg/kg, no less than 2.6 mg/kg, no less than 2.7 mg/kg, no less than 2.8 mg/kg, no less than 2.9 mg/kg, no less than 3 mg/kg, no less than 3.1 mg/kg, no less than 3.2 mg/kg, no less than 3.3 mg/kg, no less than 3.4 mg/kg, no less than 3.5 mg/kg, no less than 3.6 mg/kg, no less than 3.7 mg/kg, no less than 3.8 mg/kg, no less than 3.9 mg/kg, no less than 4 mg/kg, no less than 4.1 mg/kg, no less than 4.2 mg/kg, no less than 4.3 mg/kg, no less than 4.4 mg/kg, no less than 4.5 mg/kg, no less than 4.6 mg/kg, no less than 4.7 mg/kg, no less than 4.8 mg/kg, no less than 4.9 mg/kg, or no less than 5 mg/kg.

In other embodiments, the amount of 5HT receptor agonist, or pharmaceutically acceptable salt, solvate, metabolite, derivative, or prodrug thereof in the pharmaceutical composition corresponds to no more than 0.005 mg/kg, no more than 0.01 mg/kg, no more than 0.02 mg/kg, no more than 0.03 mg/kg, no more than 0.04 mg/kg, no more than 0.05 mg/kg, no more than 0.06 mg/kg, no more than 0.07 mg/kg, no more than 0.08 mg/kg, no more than 0.09 mg/kg, no more than 0.1 mg/kg, no more than 0.11 mg/kg, no more than 0.12 mg/kg, no more than 0.15 mg/kg, no more than 0.17 mg/kg, no more than 0.2 mg/kg, no more than 0.23 mg/kg, no more than 0.25 mg/kg, no more than 0.28 mg/kg, no more than 0.3 mg/kg, no more than 0.33 mg/kg, no more than 0.35 mg/kg, no more than 0.37 mg/kg, no more than 0.4 mg/kg, no more than 0.43 mg/kg, no more than 0.45 mg/kg, no more than 0.47 mg/kg, no more than 0.5 mg/kg, no more than 0.53 mg/kg, no more than 0.55 mg/kg, no more than 0.57 mg/kg, no more than 0.6 mg/kg, no more than 0.63 mg/kg, no more than 0.65 mg/kg, no more than 0.67 mg/kg, no more than 0.7 mg/kg, no more than 0.73 mg/kg, no more than 0.75 mg/kg, no more than 0.78 mg/kg, no more than 0.8 mg/kg, no more than 0.83 mg/kg, no more than 0.85 mg/kg, no more than 0.87 mg/kg, no more than 0.9 mg/kg, no more than 0.95 mg/kg, no more than 1 mg/kg, no more than 1.1 mg/kg, no more than 1.2 mg/kg, no more than 1.3 mg/kg, no more than 1.4 mg/kg, no more than 1.5 mg/kg, no more than 1.6 mg/kg, no more than 1.7 mg/kg, no more than 1.8 mg/kg, no more than 1.9 mg/kg, no more than 2 mg/kg, no more than 2.1 mg/kg, no more than 2.2 mg/kg, no more than 2.3 mg/kg, no more than 2.4 mg/kg, no more than 2.5 mg/kg, no more than 2.6 mg/kg, no more than 2.7 mg/kg, no more than 2.8 mg/kg, no more than 2.9 mg/kg, no more than 3 mg/kg, no more than 3.1 mg/kg, no more than 3.2 mg/kg, no more than 3.3 mg/kg, no more than 3.4 mg/kg, no more than 3.5 mg/kg, no more than 3.6 mg/kg, no more than 3.7 mg/kg, no more than 3.8 mg/kg, no more than 3.9 mg/kg, no more than 4 mg/kg, no more than 4.1 mg/kg, no more than 4.2 mg/kg, no more than 4.3 mg/kg, no more than 4.4 mg/kg, no more than 4.5 mg/kg, no more than 4.6 mg/kg, no more than 4.7 mg/kg, no more than 4.8 mg/kg, no more than 4.9 mg/kg, no more than 5 mg/kg, no more than 6 mg/kg, no more than 7 mg/kg, no more than 8 mg/kg, no more than 9 mg/kg, or no more than 10 mg/kg.

In some embodiments, the amount of 5-HT receptor agonist, or pharmaceutically acceptable salt, solvate, metabolite, derivative, or prodrugs thereof used in a pharmaceutical composition is about 1 mg/ml to about 30 mg/ml. In some embodiments, the amount of 5-HT receptor agonist, or pharmaceutically acceptable salt, solvate, metabolite, derivative, or prodrug, or prodrugs thereof used in a pharmaceutical composition is about 1 mg/ml, about 1.1 mg/ml, about 1.2 mg/ml, about 1.3 mg/ml, about 1.4 mg/ml, about 1.5 mg/ml, about 1.6 mg/ml, about 1.7 mg/ml, about 1.8 mg/ml, about 1.9 mg/ml, about 2 mg/ml, about 2.1 mg/ml, about 2.2 mg/ml, about 2.3 mg/ml, about 2.4 mg/ml, about 2.5 mg/ml, about 2.6 mg/ml, about 2.7 mg/ml, about 2.8 mg/ml, about 2.9 mg/ml, about 3 mg/ml, about 3.1 mg/ml, about 3.2 mg/ml, about 3.3 mg/ml, about 3.4 mg/ml, about 3.5 mg/ml, about 3.6 mg/ml, about 3.7 mg/ml, about 3.8 mg/ml, about 3.9 mg/ml, about 4 mg/ml, about 4.1 mg/ml, about 4.2 mg/ml, about 4.3 mg/ml, about 4.4 mg/ml, about 4.5 mg/ml, about 4.6 mg/ml, about 4.7 mg/ml, about 4.8 mg/ml, about 4.9 mg/ml, about 5 mg/ml, about 5.1 mg/ml, about 5.2 mg/ml, about 5.3 mg/ml, about 5.4 mg/ml, about 5.5 mg/ml, about 5.6 mg/ml, about 5.7 mg/ml, about 5.8 mg/ml, about 5.9 mg/ml, about 6 mg/ml, about 6.1 mg/ml, about 6.2 mg/ml, about 6.3 mg/ml, about 6.4 mg/ml, about 6.5 mg/ml, about 6.6 mg/ml, about 6.7 mg/ml, about 6.8 mg/ml, about 6.9 mg/ml, about 7 mg/ml, about 7.1 mg/ml, about 7.2 mg/ml, about 7.3 mg/ml, about 7.4 mg/ml, about 7.5 mg/ml, about 7.6 mg/ml, about 7.7 mg/ml, about 7.8 mg/ml, about 7.9 mg/ml, about 8 mg/ml, about 8.1 mg/ml, about 8.2 mg/ml, about 8.3 mg/ml, about 8.4 mg/ml, about 8.5 mg/ml, about 8.6 mg/ml, about 8.7 mg/ml, about 8.8 mg/ml, about 8.9 mg/ml, about 9 mg/ml, about 9.1 mg/ml, about 9.2 mg/ml, about 9.3 mg/ml, about 9.4 mg/ml, about 9.5 mg/ml, about 9.6 mg/ml, about 9.7 mg/ml, about 9.8 mg/ml, about 9.9 mg/ml, about 10 mg/ml, about 10.1 mg/ml, about 10.2 mg/ml, about 10.3 mg/ml, about 10.4 mg/ml, about 10.5 mg/ml, about 10.6 mg/ml, about 10.7 mg/ml, about 10.8 mg/ml, about 10.9 mg/ml, about 11 mg/ml, about 11.1 mg/ml, about 11.2 mg/ml, about 11.3 mg/ml, about 11.4 mg/ml, about 11.5 mg/ml, about 11.6 mg/ml, about 11.7 mg/ml, about 11.8 mg/ml, about 11.9 mg/ml, about 12 mg/ml, about 12.1 mg/ml, about 12.2 mg/ml, about 12.3 mg/ml, about 12.4 mg/ml, about 12.5 mg/ml, about 12.6 mg/ml, about 12.7 mg/ml, about 12.8 mg/ml, about 12.9 mg/ml, about 13 mg/ml, about 13.1 mg/ml, about 13.2 mg/ml, about 13.3 mg/ml, about 13.4 mg/ml, about 13.5 mg/ml, about 13.6 mg/ml, about 13.7 mg/ml, about 13.8 mg/ml, about 13.9 mg/ml, about 14 mg/ml, about 14.1 mg/ml, about 14.2 mg/ml, about 14.3 mg/ml, about 14.4 mg/ml, about 14.5 mg/ml, about 14.6 mg/ml, about 14.7 mg/ml, about 14.8 mg/ml, about 14.9 mg/ml, about 15 mg/ml, about 15.5 mg/ml, about 16 mg/ml, about 16.5 mg/ml, about 17 mg/ml, about 17.5 mg/ml, about 18 mg/ml, about 18.5 mg/ml, about 19 mg/ml, about 19.5 mg/ml, about 20 mg/ml, about 21 mg/ml, about 22 mg/ml, about 23 mg/ml, about 24 mg/ml, about 25 mg/ml, about 27.5 mg/ml, about 30 mg/ml.

In some embodiments, the amount of a 5-HT receptor agonist or a pharmaceutically acceptable salt, solvate, metabolite, derivative, or prodrug thereof in the pharmaceutical composition corresponds to about 0.8 mg/ml to about 24 mg/ml of the 5-HT receptor agonist or a pharmaceutically acceptable salt, solvate, metabolite, derivative, or prodrug thereof. In other embodiments, the amount of 5-HT receptor agonist, or pharmaceutically acceptable salt, solvate, metabolite, derivative, or prodrugs thereof in the pharmaceutical composition corresponds to about 0.8 mg/ml, about 0.9 mg/ml, about 1 mg/ml, about 1.1 mg/ml, about 1.2 mg/ml, about 1.3 mg/ml, about 1.4 mg/ml, about 1.5 mg/ml, about 1.6 mg/ml, about 1.7 mg/ml, about 1.8 mg/ml, about 1.9 mg/ml, about 2 mg/ml, about 2.1 mg/ml, about 2.2 mg/ml, about 2.3 mg/ml, about 2.4 mg/ml, about 2.5 mg/ml, about 2.6 mg/ml, about 2.7 mg/ml, about 2.8 mg/ml, about 2.9 mg/ml, about 3 mg/ml, about 3.1 mg/ml, about 3.2 mg/ml, about 3.3 mg/ml, about 3.4 mg/ml, about 3.5 mg/ml, about 3.6 mg/ml, about 3.7 mg/ml, about 3.8 mg/ml, about 3.9 mg/ml, about 4 mg/ml, about 4.1 mg/ml, about 4.2 mg/ml, about 4.3 mg/ml, about 4.4 mg/ml, about 4.5 mg/ml, about 4.6 mg/ml, about 4.7 mg/ml, about 4.8 mg/ml, about 4.9 mg/ml, about 5 mg/ml, about 5.1 mg/ml, about 5.2 mg/ml, about 5.3 mg/ml, about 5.4 mg/ml, about 5.5 mg/ml, about 5.6 mg/ml, about 5.7 mg/ml, about 5.8 mg/ml, about 5.9 mg/ml, about 6 mg/ml, about 6.1 mg/ml, about 6.2 mg/ml, about 6.3 mg/ml, about 6.4 mg/ml, about 6.5 mg/mi, about 6.6 mg/ml, about 6.7 mg/ml, about 6.8 mg/ml, about 6.9 mg/ml, about 7 mg/ml, about 7.1 mg/ml, about 7.2 mg/ml, about 7.3 mg/ml, about 7.4 mg/ml, about 7.5 mg/ml, about 7.6 mg/ml, about 7.7 mg/ml, about 7.8 mg/ml, about 7.9 mg/ml, about 8 mg/ml, about 8.1 mg/ml, about 8.2 mg/ml, about 8.3 mg/ml, about 8.4 mg/ml, about 8.5 mg/ml, about 8.6 mg/ml, about 8.7 mg/ml, about 8.8 mg/ml, about 8.9 mg/ml, about 9 mg/ml, about 9.1 mg/ml, about 9.2 mg/ml, about 9.3 mg/ml, about 9.4 mg/ml, about 9.5 mg/ml, about 9.6 mg/ml, about 9.7 mg/ml, about 9.8 mg/ml, about 9.9 mg/ml, about 10 mg/ml, about 10.1 mg/ml, about 10.2 mg/ml, about 10.3 mg/ml, about 10.4 mg/ml, about 10.5 mg/ml, about 10.6 mg/ml, about 10.7 mg/ml, about 10.8 mg/ml, about 10.9 mg/ml, about 11 mg/ml, about 11.1 mg/ml, about 11.2 mg/ml, about 11.3 mg/ml, about 11.4 mg/ml, about 11.5 mg/ml, about 11.6 mg/ml, about 11.7 mg/ml, about 11.8 mg/ml, about 11.9 mg/ml, about 12 mg/ml, about 12.1 mg/ml, about 12.2 mg/ml, about 12.3 mg/ml, about 12.4 mg/ml, about 12.5 mg/ml, about 12.6 mg/ml, about 12.7 mg/ml, about 12.8 mg/ml, about 12.9 mg/ml, about 13 mg/ml, about 13.1 mg/ml, about 13.2 mg/ml, about 13.3 mg/ml, about 13.4 mg/ml, about 13.5 mg/ml, about 13.6 mg/ml, about 13.7 mg/ml, about 13.8 mg/ml, about 13.9 mg/ml, about 14 mg/ml, about 14.1 mg/ml, about 14.2 mg/ml, about 14.3 mg/ml, about 14.4 mg/ml, about 14.5 mg/ml, about 14.6 mg/ml, about 14.7 mg/ml, about 14.8 mg/ml, about 14.9 mg/ml, about 15 mg/ml, about 15.5 mg/ml, about 16 mg/ml, about 16.5 mg/ml, about 17 mg/ml, about 17.5 mg/ml, about 18 mg/ml, about 18.5 mg/ml, about 19 mg/ml, about 19.5 mg/ml, about 20 mg/ml, about 21 mg/ml, about 22 mg/ml, about 23 mg/ml, or about 24 mg/ml of a 5-HT receptor agonist or a pharmaceutically acceptable salt, solvate, metabolite, derivative, or prodrug thereof.

In some embodiments, the amount of 5-HT receptor agonist, or pharmaceutically acceptable salt, solvate, metabolite, derivative, or prodrugs thereof in the pharmaceutical composition corresponds to about 1% w/w to about 50% w/w of the solids in the oral formulation. In other embodiments, the amount of the pharmaceutically acceptable salt of a 5-HT receptor agonist or a pharmaceutically acceptable salt, solvate, metabolite, derivative, or prodrug thereof correspond to about 1% w/w, about 1.1% w/w, about 1.2% w/w, about 1.3% w/w, about 1.4% w/w, about 1.5% w/w, about 1.6% w/w, about 1.7% w/w, about 1.8% w/w, about 1.9% w/w, about 2% w/w, about 2.1% w/w, about 2.2% w/w, about 2.3% w/w, about 2.4% w/w, about 2.5% w/w, about 2.6% w/w, about 2.7% w/w, about 2.8% w/w, about 2.9% w/w, about 3% w/w, about 3.1% w/w, about 3.2% w/w, about 3.3% w/w, about 3.4% w/w, about 3.5% w/w, about 3.6% w/w, about 3.7% w/w, about 3.8% w/w, about 3.9% w/w, about 4% w/w, about 4.1% w/w, about 4.2% w/w, about 4.3% w/w, about 4.4% w/w, about 4.5% w/w, about 4.6% w/w, about 4.7% w/w, about 4.8% w/w, about 4.9% w/w, about 5% w/w, about 5.1% w/w, about 5.2% w/w, about 5.3% w/w, about 5.4% w/w, about 5.5% w/w, about 5.6% w/w, about 5.7% w/w, about 5.8% w/w, about 5.9% w/w, about 6% w/w, about 6.1% w/w, about 6.2% w/w, about 6.3% w/w, about 6.4% w/w, about 6.5% w/w, about 6.6% w/w, about 6.7% w/w, about 6.8% w/w, about 6.9% w/w, about 7% w/w, about 7.1% w/w, about 7.2% w/w, about 7.3% w/w, about 7.4% w/w, about 7.5% w/w, about 7.6% w/w, about 7.7% w/w, about 7.8% w/w, about 7.9% w/w, about 8% w/w, about 8.1% w/w, about 8.2% w/w, about 8.3% w/w, about 8.4% w/w, about 8.5% w/w, about 8.6% w/w, about 8.7% w/w, about 8.8% w/w, about 8.9% w/w, about 9% w/w, about 9.1% w/w, about 9.2% w/w, about 9.3% w/w, about 9.4% w/w, about 9.5% w/w, about 9.6% w/w, about 9.7% w/w, about 9.8% w/w, about 9.9% w/w, about 10% w/w, about 10.2% w/w, about 10.4% w/w, about 10.6% w/w, about 10.8% w/w, about 11% w/w, about 11.2% w/w, about 11.4% w/w, about 11.6% w/w, about 11.8% w/w, about 12% w/w, about 12.2% w/w, about 12.4% w/w, about 12.6% w/w, about 12.8% w/w, about 13% w/w, about 13.2% w/w, about 13.4% w/w, about 13.6% w/w, about 13.8% w/w, about 14% w/w, about 14.2% w/w, about 14.4% w/w, about 14.6% w/w, about 14.8% w/w, about 15% w/w, about 15.5% w/w, about 16% w/w, about 16.5% w/w, about 17% w/w, about 17.5% w/w, about 18% w/w, about 18.5% w/w, about 19% w/w, about 19.5% w/w, about 20% w/w, about 21% w/w, about 22% w/w, about 23% w/w, about 24% w/w, about 25% w/w, about 26% w/w, about 27% w/w, about 28% w/w, about 29% w/w, about 30% w/w, about 31% w/w, about 32% w/w, about 33% w/w, about 34% w/w, about 35% w/w, about 36% w/w, about 37% w/w, about 38% w/w, about 39% w/w, about 40% w/w, about 41% w/w, about 42% w/w, about 43% w/w, about 44% w/w, about 45% w/w, about 46% w/w, about 47% w/w, about 48% w/w, about 49% w/w, or about 50% w/w of the solids in the oral liquid formulation.

### Therapeutic Uses—Disorders, Conditions and Symptoms

Provided herein are methods for managing disorders or conditions, comprising administering one or more 5-HT receptor agonists, or pharmaceutically acceptable salts, solvates, metabolites, derivatives, or prodrugs thereof.

Further provided herein are methods for treating symptoms of disorders or conditions, comprising administering one or more 5-HT receptor agonists, or pharmaceutically acceptable salts, solvates, metabolites, derivatives, or prodrugs thereof.

Provided in some embodiments herein is a method for treating or managing a mental, a behavioral, or a neuropsychiatric condition, or the symptoms thereof, comprising administering one or more 5-HT receptor agonists, or pharmaceutically acceptable salts, solvates, metabolites, derivatives, or prodrugs thereof.

In certain instances, provided herein is a method for treating or managing a mental, a behavioral, or a neuropsychiatric condition, or the symptoms thereof, in an individual in need thereof (e.g., in an individual suffering from or susceptible to the mental, a behavioral, or a neuropsychiatric condition), comprising: (a) administering to the individual a therapeutically effective amount of one or more 5-hydroxytryptamine (5-HT) receptor agonist, or a pharmaceutically acceptable salt, solvate, metabolite, derivative, or prodrug thereof; and (b) maintaining a level of an active 5-HT receptor agonist (i) at or above a minimum therapeutically effective threshold and (ii) below a hallucinogenic threshold (e.g., below a threshold that produces an adverse event (e.g., a clinically important effect (e.g., clinically important impairment of the individual), altered (e.g., visual, auditory, body, time and space) perception, altered cognition, impaired attention, drowsiness, confusion, or the like) of the active 5-HT receptor agonist in the individual (e.g., in a biological sample (e.g., serum, plasma, or whole blood) of the individual) for more than or equal to two hours (e.g., more than 2 hours, more than 12 hours, more than 1 day, more than 7 days, or more than 14 days).

In certain embodiments, provided herein is method for treating or managing a mental, a behavioral, or a neuropsychiatric condition, or the symptoms thereof, in an individual in need thereof (e.g., in an individual suffering from or susceptible to the mental, a behavioral, or a neuropsychiatric condition), comprising (a) administering to the individual a therapeutically effective amount of one or more 5-hydroxytryptamine (5-HT) receptor agonist, or a pharmaceutically acceptable salt, solvate, metabolite, derivative, or prodrug thereof at a first time; and (b) administering to the individual a therapeutically effective amount of one or more 5-hydroxytryptamine (5-HT) receptor agonist, or a pharmaceutically acceptable salt, solvate, metabolite, derivative, or prodrug thereof at a second time (e.g., wherein the second time is at least one day after the first time, at least 2 days after the first time, at least 3 days after the first time, at least 4 days after the first time, at least 7 days after the first time (e.g., with optional intervening and/or subsequent administration of a therapeutically effective amount of one or more 5-hydroxytryptamine (5-HT) receptor agonist) (e.g., wherein the second administration of a therapeutically effective amount of one or more 5-hydroxytryptamine (5-HT) receptor agonist is the same or different from the first administration of a therapeutically effective amount of one or more 5-hydroxytryptamine (5-HT) receptor agonist).

In some embodiments, the first time and the second time are within a 24 hour period (e.g., within a 4 hour period, within a 6 hour period, within an 8 hour period, within a 12 hour period, or the like). In some embodiments, the first time and the second time are on or after a 24 hour period (e.g., the first time is more than or equal to 24 hours after the second time, the first time is more than or equal to 48 hours after the second time, or the like).

In some embodiments, the method further comprises administering to the individual the therapeutically effective amount of one or more 5-HT receptor agonist, or a pharmaceutically acceptable salt, solvate, metabolite, derivative, or prodrug thereof a third time, wherein the third time is a time between the first time and the second time or after the second time.

In some embodiments, the method further comprises administering to the individual the therapeutically effective amount of one or more 5-HT receptor agonist, or a pharmaceutically acceptable salt, solvate, metabolite, derivative, or prodrug thereof a fourth time. In some embodiments, the fourth time is a time between the first and third time. In some embodiments, the fourth time is a time between the first and second time. In some embodiments, the fourth time is a time after the first time. In some embodiments, the fourth time is a time after the second time. In some embodiments, the fourth time is a time after the third time.

In some embodiments, the mental, the behavioral, or the neuropsychiatric condition is an attention condition or a cognitive (e.g., neurocognitive) condition. In some embodiments, the mental, the behavioral, or the neuropsychiatric condition (e.g., a Diagnostic and Statistical Manual of Mental Disorders (DSM-5) category or non-DSM-5 category disease or disorder) is selected from the group consisting of addiction (e.g., a weight management disorder), anxiety (e.g., post-traumatic stress disorder (PTSD), constructive impulsivity, a phobia, or fear), apathy, and depression (e.g., major depressive disorder, moderate depression, prolonged grief disorder (PGD), social anxiety, post-surgical depression, or depression from chronic pain).

In some embodiments, the symptoms of the mental, a behavioral, or a neuropsychiatric condition are physical, behavioral, emotional, mental, or a combination thereof.

Provided in some instances herein is a method for increasing motivation in an individual (e.g., in an individual suffering from or susceptible to low motivation (e.g., as a symptom of a neurocognitive or neurodevelopmental disorder), apathy, fear, phobia, constructive impulsivity, attention (e.g., or the lack thereof), cognitive conditions, or depression), comprising administering one or more 5-HT receptor agonists, or pharmaceutically acceptable salts, solvates, metabolites, derivatives, or prodrugs thereof.

In certain instances, provided herein is a method for increasing motivation in an individual (e.g., in an individual suffering from or susceptible to low motivation, anxiety, apathy, fear, phobia, constructive impulsivity, or depression), comprising: (a) administering to the individual a therapeutically effective amount of one or more 5-hydroxytryptamine (5-HT) receptor agonist, or a pharmaceutically acceptable salt, solvate, metabolite, derivative, or prodrug thereof; and maintaining a level of an active 5-HT receptor agonist (i) at or above a minimum therapeutically effective threshold and (ii) below a hallucinogenic threshold (e.g., below a threshold that produces an adverse event (e.g., a clinically important effect (e.g., clinically important impairment of the individual), altered (e.g., visual, auditory, body, time and space) perception, altered cognition, impaired attention, drowsiness, confusion, or the like) of the active 5-HT receptor agonist in the individual (e.g., in a biological sample (e.g., serum, plasma, or whole blood) of the individual) for more than or equal to two hours (e.g., more than 2 hours, more than 12 hours, more than 1 day, more than 7 days, or more than 14 days).

In some embodiments, the method comprises administering to the individual the therapeutically effective amount of one or more 5-HT receptor agonist, or a pharmaceutically acceptable salt, solvate, metabolite, derivative, or prodrug thereof, active 5-HT receptor agonist at a dose sufficient to provide a maximum plasma concentration (Cmax) Cmax of the active 5-HT receptor agonist below the hallucinogenic effective threshold of the active 5-HT receptor agonist and a minimum plasma concentration (Cₘᵢₙ) of the active 5-HT receptor agonist of at least the therapeutically effective threshold of the active 5-HT receptor agonist in the individual.

In some embodiments, the level of the active 5-HT receptor agonist is maintained in the individual (e.g., in a biological sample (e.g., serum, plasma, or whole blood) of the individual) above the therapeutically effective threshold and below the hallucinogenic effective threshold of the active 5-HT receptor agonist. In some embodiments, the level of the active 5-HT receptor agonist in the individual (e.g., in a biological sample (e.g., serum, plasma, or whole blood) of the individual) is from about 0.01 ng/mL to about 10 ng/mL. In some embodiments, the level of the active 5-HT receptor agonist in the individual (e.g., in a biological sample (e.g., serum, plasma, or whole blood) of the individual) is from about 0.1 ng/mL to about 2.0 ng/mL.

In some embodiments, wherein the therapeutically effective amount of the one or more 5-HT receptor agonist, or a pharmaceutically acceptable salt, solvate, metabolite, derivative, or prodrug thereof, is administered to the individual in need thereof in an amount and/or formulation insufficient to provide a Cₘₐₓ of the active 5-HT receptor agonist of 6 ng/mL or more in the individual. In some embodiments, the therapeutically effective amount of the one or more 5-HT receptor agonist, or a pharmaceutically acceptable salt, solvate, metabolite, derivative, or prodrug thereof, is administered to the individual in need thereof in an amount and/or formulation to provide a Cₘₐₓ of the active 5-HT receptor agonist of about 0.1 ng/mL to about 6 ng/mL (e.g., about 0.5 ng/mL to about 6 ng/mL, about 1 ng/mL to about 5.5 ng/mL, about 2 ng/mL to about 5 ng/mL, or the like) in the individual.

In some embodiments, the therapeutically effective amount of the 5HT receptor agonist or a pharmaceutically acceptable salt, solvate, metabolite, derivative, or prodrug thereof is provided to a subject in need thereof in an amount and/or formulation insufficient to provide a maximum plasma concentration (Cₘₐₓ) of (e.g. active form of the) 5HT receptor agonist or a pharmaceutically acceptable salt, solvate, metabolite, derivative, or prodrug thereof of at least 0.001 ng/mL (e.g., 0.001 ng/mL or more, 0.01 ng/mL or more, 0.1 ng/mL or more, ng/mL or more, 10 ng/mL or more, or 100 ng/mL or more). In some embodiments, the therapeutically effective amount of the 5HT receptor agonist or a pharmaceutically acceptable salt, solvate, metabolite, derivative, or prodrug thereof is provided to a subject in need thereof in an amount and/or formulation insufficient to provide a maximum plasma concentration (Cₘₐₓ) of (e.g. active form of the) 5HT receptor agonist or a pharmaceutically acceptable salt, solvate, metabolite, derivative, or prodrug thereof of at most 100 ng/mL (e.g., 100 ng/mL or less, 10 ng/mL or less, 1 ng/mL or less, 0.1 ng/mL or less, 0.01 ng/mL or less, or 0.001 ng/mL or less). In some embodiments, the therapeutically effective amount of the 5HT receptor agonist or a pharmaceutically acceptable salt, solvate, metabolite, derivative, or prodrug thereof is provided to a subject in need thereof in an amount and/or formulation insufficient to provide a maximum plasma concentration (Cₘₐₓ) of (e.g. active form of the) 5HT receptor agonist or a pharmaceutically acceptable salt, solvate, metabolite, derivative, or prodrug thereof of about 0.001 ng/mL to about 100 ng/mL (e.g., about 0.01 ng/mL to about 100 ng/mL, about 0.01 ng/mL to about 50 ng/mL, or about 0.1 ng/mL to about 50 ng/mL).

In some embodiments, the therapeutically effective amount of 5HT receptor agonist or a pharmaceutically acceptable salt, solvate, metabolite, derivative, or prodrug thereof is provided to a subject in need thereof in an amount and/or formulation insufficient to provide a maximum plasma concentration (Cₘₐₓ) of (e.g. active form of the) 5HT receptor agonist or a pharmaceutically acceptable salt, solvate, metabolite, derivative, or prodrug thereof of about 0.01 ng/mL to about 5 ng/mL. In some embodiments, the therapeutically effective amount of 5HT receptor agonist or a pharmaceutically acceptable salt, solvate, metabolite, derivative, or prodrug thereof is provided to a subject in need thereof in an amount and/or formulation insufficient to provide a maximum plasma concentration (Cₘₐₓ) of (e.g. active form of the) 5HT receptor agonist or a pharmaceutically acceptable salt, solvate, metabolite, derivative, or prodrug thereof of about 0.05 ng/mL to about 1 ng/mL.

In other embodiments, the therapeutically effective amount of 5HT receptor agonist or a pharmaceutically acceptable salt, solvate, metabolite, derivative, or prodrug thereof is provided to a subject in need thereof in an amount and/or formulation insufficient to provide a maximum plasma concentration (Cₘₐₓ) of (e.g. active form of the) 5HT receptor agonist or a pharmaceutically acceptable salt, solvate, metabolite, derivative, or prodrug thereof of about 0.001 ng/mL, about 0.005 ng/mL, about 0.01 ng/mL, about 0.02 ng/mL, about 0.03 ng/mL, about 0.04 ng/mL, about 0.05 ng/mL, about 0.06 ng/mL, about 0.07 ng/mL, about 0.08 ng/mL, about 0.09 ng/mL, about 0.1 ng/mL, about 0.11 ng/mL, about 0.12 ng/mL, about 0.15 ng/mL, about 0.17 ng/mL, about 0.2 ng/mL, about 0.23 ng/mL, about 0.25 ng/mL, about 0.28 ng/mL, about 0.3 ng/mL, about 0.33 ng/mL, about 0.35 ng/mL, about 0.37 ng/mL, about 0.4 ng/mL, about 0.43 ng/mL, about 0.45 ng/mL, about 0.47 ng/mL, about 0.5 ng/mL, about 0.53 ng/mL, about 0.55 ng/mL, about 0.57 ng/mL, about 0.6 ng/mL, about 0.63 ng/mL, about 0.65 ng/mL, about 0.67 ng/mL, about 0.7 ng/mL, about 0.73 ng/mL, about 0.75 ng/mL, about 0.78 ng/mL, about 0.8 ng/mL, about 0.83 ng/mL, about 0.85 ng/mL, about 0.87 ng/mL, about 0.9 ng/mL, about 0.95 ng/mL, about 1 ng/mL, about 1.1 ng/mL, about 1.2 ng/mL, about 1.3 ng/mL, about 1.4 ng/mL, about 1.5 ng/mL, about 1.6 ng/mL, about 1.7 ng/mL, about 1.8 ng/mL, about 1.9 ng/mL, about 2 ng/mL, about 2.1 ng/mL, about 2.2 ng/mL, about 2.3 ng/mL, about 2.4 ng/mL, about 2.5 ng/mL, about 2.6 ng/mL, about 2.7 ng/mL, about 2.8 ng/mL, about 2.9 ng/mL, about 3 ng/mL, about 3.1 ng/mL, about 3.2 ng/mL, about 3.3 ng/mL, about 3.4 ng/mL, about 3.5 ng/mL, about 3.6 ng/mL, about 3.7 ng/mL, about 3.8 ng/mL, about 3.9 ng/mL, about 4 ng/mL, about 4.1 ng/mL, about 4.2 ng/mL, about 4.3 ng/mL, about 4.4 ng/mL, about 4.5 ng/mL, about 4.6 ng/mL, about 4.7 ng/mL, about 4.8 ng/mL, about 4.9 ng/mL, about 5 ng/mL, about 5.1 ng/mL, about 5.2 ng/mL, about 5.3 ng/mL, about 5.4 ng/mL, about 5.5 ng/mL, about 5.6 ng/mL, about 5.7 ng/mL, about 5.8 ng/mL, about 5.9 ng/mL, or about 6 ng/mL.

In other embodiments, the therapeutically effective amount of 5HT receptor agonist or a pharmaceutically acceptable salt, solvate, metabolite, derivative, or prodrug thereof is provided to a subject in need thereof in an amount and/or formulation insufficient to provide a maximum plasma concentration (Cₘₐₓ) of (e.g. active form of the) 5HT receptor agonist or a pharmaceutically acceptable salt, solvate, metabolite, derivative, or prodrug thereof of no less than 0.001 ng/mL, no less than 0.005 ng/mL, no less than 0.01 ng/mL, no less than 0.02 ng/mL, no less than 0.03 ng/mL, no less than 0.04 ng/mL, no less than 0.05 ng/mL, no less than 0.06 ng/mL, no less than 0.07 ng/mL, no less than 0.08 ng/mL, no less than 0.09 ng/mL, no less than 0.1 ng/mL, no less than 0.11 ng/mL, no less than 0.12 ng/mL, no less than 0.15 ng/mL, no less than 0.17 ng/mL, no less than 0.2 ng/mL, no less than 0.23 ng/mL, no less than 0.25 ng/mL, no less than 0.28 ng/mL, no less than 0.3 ng/mL, no less than 0.33 ng/mL, no less than 0.35 ng/mL, no less than 0.37 ng/mL, no less than 0.4 ng/mL, no less than 0.43 ng/mL, no less than 0.45 ng/mL, no less than 0.47 ng/mL, no less than 0.5 ng/mL, no less than 0.53 ng/mL, no less than 0.55 ng/mL, no less than 0.57 ng/mL, no less than 0.6 ng/mL, no less than 0.63 ng/mL, no less than 0.65 ng/mL, no less than 0.67 ng/mL, no less than 0.7 ng/mL, no less than 0.73 ng/mL, no less than 0.75 ng/mL, no less than 0.78 ng/mL, no less than 0.8 ng/mL, no less than 0.83 ng/mL, no less than 0.85 ng/mL, no less than 0.87 ng/mL, no less than 0.9 ng/mL, no less than 0.95 ng/mL, no less than 1 ng/mL, no less than 1.1 ng/mL, no less than 1.2 ng/mL, no less than 1.3 ng/mL, no less than 1.4 ng/mL, no less than 1.5 ng/mL, no less than 1.6 ng/mL, no less than 1.7 ng/mL, no less than 1.8 ng/mL, no less than 1.9 ng/mL, no less than 2 ng/mL, no less than 2.1 ng/mL, no less than 2.2 ng/mL, no less than 2.3 ng/mL, no less than 2.4 ng/mL, no less than 2.5 ng/mL, no less than 2.6 ng/mL, no less than 2.7 ng/mL, no less than 2.8 ng/mL, no less than 2.9 ng/mL, no less than 3 ng/mL, no less than 3.1 ng/mL, no less than 3.2 ng/mL, no less than 3.3 ng/mL, no less than 3.4 ng/mL, no less than 3.5 ng/mL, no less than 3.6 ng/mL, no less than 3.7 ng/mL, no less than 3.8 ng/mL, no less than 3.9 ng/mL, no less than 4 ng/mL, no less than 4.1 ng/mL, no less than 4.2 ng/mL, no less than 4.3 ng/mL, no less than 4.4 ng/mL, no less than 4.5 ng/mL, no less than 4.6 ng/mL, no less than 4.7 ng/mL, no less than 4.8 ng/mL, no less than 4.9 ng/mL, no less than 5 ng/mL, no less than 5.1 ng/mL, no less than 5.2 ng/mL, no less than 5.3 ng/mL, no less than 5.4 ng/mL, no less than 5.5 ng/mL, no less than 5.6 ng/mL, no less than 5.7 ng/mL, no less than 5.8 ng/mL, or no less than 5.9 ng/mL.

In other embodiments, the therapeutically effective amount of 5HT receptor agonist or a pharmaceutically acceptable salt, solvate, metabolite, derivative, or prodrug thereof is provided to a subject in need thereof in an amount and/or formulation insufficient to provide a maximum plasma concentration (Cₘₐₓ) of (e.g. active form of the) 5HT receptor agonist or a pharmaceutically acceptable salt, solvate, metabolite, derivative, or prodrug thereof of no more than 0.005 ng/mL, no more than 0.01 ng/mL, no more than 0.02 ng/mL, no more than 0.03 ng/mL, no more than 0.04 ng/mL, no more than 0.05 ng/mL, no more than 0.06 ng/mL, no more than 0.07 ng/mL, no more than 0.08 ng/mL, no more than 0.09 ng/mL, no more than 0.1 ng/mL, no more than 0.11 ng/mL, no more than 0.12 ng/mL, no more than 0.15 ng/mL, no more than 0.17 ng/mL, no more than 0.2 ng/mL, no more than 0.23 ng/mL, no more than 0.25 ng/mL, no more than 0.28 ng/mL, no more than 0.3 ng/mL, no more than 0.33 ng/mL, no more than 0.35 ng/mL, no more than 0.37 ng/mL, no more than 0.4 ng/mL, no more than 0.43 ng/mL, no more than 0.45 ng/mL, no more than 0.47 ng/mL, no more than 0.5 ng/mL, no more than 0.53 ng/mL, no more than 0.55 ng/mL, no more than 0.57 ng/mL, no more than 0.6 ng/mL, no more than 0.63 ng/mL, no more than 0.65 ng/mL, no more than 0.67 ng/mL, no more than 0.7 ng/mL, no more than 0.73 ng/mL, no more than 0.75 ng/mL, no more than 0.78 ng/mL, no more than 0.8 ng/mL, no more than 0.83 ng/mL, no more than 0.85 ng/mL, no more than 0.87 ng/mL, no more than 0.9 ng/mL, no more than 0.95 ng/mL, no more than 1 ng/mL, no more than 1.1 ng/mL, no more than 1.2 ng/mL, no more than 1.3 ng/mL, no more than 1.4 ng/mL, no more than 1.5 ng/mL, no more than 1.6 ng/mL, no more than 1.7 ng/mL, no more than 1.8 ng/mL, no more than 1.9 ng/mL, no more than 2 ng/mL, no more than 2.1 ng/mL, no more than 2.2 ng/mL, no more than 2.3 ng/mL, no more than 2.4 ng/mL, no more than 2.5 ng/mL, no more than 2.6 ng/mL, no more than 2.7 ng/mL, no more than 2.8 ng/mL, no more than 2.9 ng/mL, no more than 3 ng/mL, no more than 3.1 ng/mL, no more than 3.2 ng/mL, no more than 3.3 ng/mL, no more than 3.4 ng/mL, no more than 3.5 ng/mL, no more than 3.6 ng/mL, no more than 3.7 ng/mL, no more than 3.8 ng/mL, no more than 3.9 ng/mL, no more than 4 ng/mL, no more than 4.1 ng/mL, no more than 4.2 ng/mL, no more than 4.3 ng/mL, no more than 4.4 ng/mL, no more than 4.5 ng/mL, no more than 4.6 ng/mL, no more than 4.7 ng/mL, no more than 4.8 ng/mL, no more than 4.9 ng/mL, no more than 5 ng/mL, no more than 5.1 ng/mL, no more than 5.2 ng/mL, no more than 5.3 ng/mL, no more than 5.4 ng/mL, no more than 5.5 ng/mL, no more than 5.6 ng/mL, no more than 5.7 ng/mL, no more than 5.8 ng/mL, no more than 5.9 ng/mL, or no more than 6 ng/mL.

In some embodiments, the therapeutically effective amount of the one or more 5-HT receptor agonist, or a pharmaceutically acceptable salt, solvate, metabolite, derivative, or prodrug thereof, is administered to the individual in need thereof in an amount and/or formulation to provide a plasma concentration of the active 5-HT receptor agonist of at least 0.1 ng/mL (e.g., at least 0.2 ng/mL, at least 0.3 ng/mL, at least 0.5 ng/mL, or the like) in the individual for at least 3 hours (e.g., at least 12 hours, at least 24 hours, at least 36 hours, at least 48 hours, at least 72 hours, at least 96 hours, at least 120 hours, at least 144 hours, or the like).

In some embodiments, the level (e.g., Cₘₐₓ) of the active 5-HT receptor agonist (e.g., psilocin) in the individual is (maintained) at a level of at least about 0.001 ng/mL or more (e.g., 0.01 ng/mL or more, 0.1 ng/mL or more, 1 ng/mL or more, 10 ng/mL or more, 20 ng/mL or more, or 50 ng/mL or more). In some embodiments, the level (e.g., Cₘₐₓ) of the active 5-HT receptor agonist (e.g., psilocin) in the individual is (maintained) at a level of at least about 100 ng/mL or less (e.g., 50 ng/mL or less, 25 ng/mL or less, 15 ng/mL or less, 5 ng/mL or less, or 0.5 ng/mL or less). In some embodiments, the level (e.g., Cₘₐₓ) of the active 5-HT receptor agonist (e.g., psilocin) in the individual is (maintained) at a level of about 0.001 ng/mL to about 100 ng/mL. In some embodiments, the level (e.g., Cₘₐₓ) of the active 5-HT receptor agonist (e.g., psilocin) in the individual is (maintained) at a level of about 0.1 ng/mL to about 50 ng/mL. In some embodiments, the level (e.g., Cₘₐₓ) of the active 5-HT receptor agonist (e.g., psilocin) in the individual is (maintained) at a level of about 1 ng/mL to about 25 ng/mL. In some embodiments, the level (e.g., Cₘₐₓ) of the active 5-HT receptor agonist (e.g., psilocin) in the individual is (maintained) at a level of about 2 ng/mL to about 12 ng/mL. In some embodiments, the level (e.g., Cₘₐₓ) of the active 5-HT receptor agonist (e.g., psilocin) in the individual is (maintained) at a level of about 5 ng/mL to about 24 ng/mL. In some embodiments, the level (e.g., Cₘₐₓ) is measured after a dose of psilocybin (e.g., a dose provided herein) is administered to the individual. In some embodiments, the level (e.g., Cₘₐₓ) of the active 5-HT receptor agonist (e.g., psilocin) is a sub-hallucinogenic level of the active 5-HT receptor agonist (e.g., psilocin). In some embodiments, the level (e.g., Cₘₐₓ) of the active 5-HT receptor agonist (e.g., psilocin) is measured after a dose of at least 1 mg or more (e.g., 5 mg or more, 10 mg or more, 15 mg or more, or 20 mg or more) of the 5-HT receptor agonist (e.g., psilocybin) is administered to the individual. In some embodiments, the dose of the 5-HT receptor agonist (e.g., psilocybin) is a sub-hallucinogenic dose of the 5-HT receptor agonist (e.g., psilocybin) or the active 5-HT receptor agonist (e.g., psilocin) In some embodiments, psilocybin is administered orally. In some embodiments, is administered intravenously.

In some instances, a dose, pharmacokinetic parameter, or the like may change, such as, when using different formulations (e.g., as provided herein). In some instances, a dose, pharmacokinetic parameter, or the like may change, such as, when using different formulations (e.g., as provided herein), but the dose, pharmacokinetic parameter, or the like is generically sub-hallucinogenic. In some instances, a dose, pharmacokinetic parameter, or the like may change, such as, when using different formulations (e.g., as provided herein), but the dose, pharmacokinetic parameter, or the like is generically therapeutically effective and sub-hallucinogenic.

In some embodiments, the therapeutically effective amount of the one or more 5-HT receptor agonist, or a pharmaceutically acceptable salt, solvate, metabolite, derivative, or prodrug thereof, is administered to the individual in need thereof over an extended period of time (e.g., daily for a week, every other day for a week, two times a week, once a week, bi-weekly, or the like).

In some embodiments, the therapeutically effective amount of the one or more 5-HT receptor agonist, or a pharmaceutically acceptable salt, solvate, metabolite, derivative, or prodrug thereof, is administered to the individual in need thereof as a controlled release formulation (e.g., that releases the active 5-HT receptor agonist (e.g., at a Cₘₐₓ below the hallucinogenic effective threshold and a Cₘᵢₙ of at least the therapeutically effective threshold)). In some embodiments, the controlled release formulation comprises an extended release component (e.g., that releases the active 5-HT receptor agonist (e.g., at a Cₘₐₓ below the hallucinogenic effective threshold and a Cₘᵢₙ of at least the therapeutically effective threshold)). In some embodiments, the controlled release formulation comprises an extended release component (e.g., that releases the active 5-HT receptor agonist (e.g., at a Cₘₐₓ below the hallucinogenic effective threshold and a Cₘᵢₙ of at least the therapeutically effective threshold)) and an immediate release component (e.g., that releases the active 5-HT receptor agonist (e.g., at a Cₘₐₓ below the hallucinogenic effective threshold and a Cₘᵢₙ of at least the therapeutically effective threshold)).

In some embodiments, the controlled release formulation is as described herein. In some embodiments, the immediate release formulation is as described herein.

In some embodiments, the immediate release component is an immediate-release coating (e.g., providing immediate release of the active 5-HT receptor agonist). In some embodiments, the immediate-release coating surrounds the controlled release component (e.g., wherein the immediate-release coating and the controlled release component each release the active 5-HT receptor agonist).

In some embodiments, the controlled release formulation is an oral formulation, a dermal formulation, a buccal formulation, a nasal formulation, or an inhalation formulation. In some embodiments, the oral formulation is in a solid form or a liquid form.

In some embodiments, the controlled release formulation releases the active 5-HT receptor agonist in the individual in need thereof for a period of at least two hours. In some embodiments, the controlled release formulation releases the active 5-HT receptor agonist in the individual in need thereof for a period of at least one day. In some embodiments, the controlled release formulation is released in the individual in need thereof for a period of two hours to one week.

In some embodiments, the one or more 5-HT receptor agonist, or the pharmaceutically acceptable salt, solvate, metabolite, derivative, or prodrug thereof, is a hallucinogenic compound (e.g., wherein the hallucinogenic compound produces a hallucinogenic effect (e.g., an adverse event, a clinically important effect (e.g., clinically important impairment of the individual, altered (e.g., visual, auditory, body, time and space) perception, altered cognition, impaired attention, drowsiness, and/or confusion)) in the individual in need thereof at or above the hallucinogenic threshold (e.g., at or above a Cₘₐₓ above the hallucinogenic effective threshold)).

In some embodiments, the one or more 5-HT receptor agonist, or a pharmaceutically acceptable salt, solvate, metabolite, derivative, or prodrug thereof is a 5HT_{2A} and/or 5HT_{2C} receptor agonist. In some embodiments, the one or more 5-HT receptor agonist, or a pharmaceutically acceptable salt, solvate, metabolite, derivative, or prodrug thereof is a 5HT_{2C} receptor agonist. In some embodiments, the one or more 5-HT receptor agonist, or a pharmaceutically acceptable salt, solvate, metabolite, derivative, or prodrug thereof is a 5HT_{2C} receptor agonist. In some embodiments, the one or more 5-HT receptor agonist, or a pharmaceutically acceptable salt, solvate, metabolite, derivative, or prodrug thereof is a 5HT_{2A} and 5HT_{2C} receptor agonist.

In some embodiments, the one or more 5-HT receptor agonist is selected from the group consisting of LSD, dimethyltryptamine (DMT), psilocybin, and psilocin, or a pharmaceutically acceptable salt, solvate, metabolite, derivative, or prodrug thereof. In some embodiments, the one or more 5-HT receptor agonist is psilocybin or psilocin, or a pharmaceutically acceptable salt, solvate, metabolite, derivative, or prodrug thereof. In some embodiments, the active 5-HT receptor agonist is psilocin. In some embodiments, the active 5-HT receptor agonist is psilocybin.

In some embodiments, the disorders or conditions are neurological disorders or conditions. In some embodiments, the disorders or conditions are neurocognitive disorders or conditions. In some embodiments, the disorders or conditions are neurodegenerative disorders or conditions. In some embodiments, the symptoms of the neurological condition are physical, behavioral, emotional, mental, or a combination thereof.

In some embodiments, the mental, the behavioral, or the neuropsychiatric condition is depression or a depression disorder (e.g., prolonged grief disorder (PGD), clinical depression, post-surgical depression, depression from chronic pain). In some embodiments, the mental, the behavioral, or the neuropsychiatric condition is prolonged grief disorder (PGD). In some embodiments, the mental, the behavioral, or the neuropsychiatric condition is social anxiety. In some embodiments, the mental, the behavioral, or the neuropsychiatric condition is post-surgical depression. In some embodiments, the mental, the behavioral, or the neuropsychiatric condition is depression from chronic pain.

In some embodiments, the mental, the behavioral, or the neuropsychiatric condition is related to COVID-19 (e.g., a mental condition (e.g., anxiety, depression, or the like) that developed after and/or from the COVID-19 pandemic).

In some embodiments, the disorder is a body weight management disorder. In some embodiments, the individual administered the 5-HT receptor agonist (described herein) maintains a certain (e.g., desired) body weight. In some embodiments, the individual administered the 5-HT receptor agonist (described herein) reduces weight gain. In some embodiments, the individual administered the 5-HT receptor agonist (described herein) increases weight loss (e.g., through reducing food cravings). In some embodiments, the individual administered the 5-HT receptor agonist (described herein) diminishes food addition. In some embodiments, the individual administered the 5-HT receptor agonist (described herein) decreases impulsive eating. In some embodiments, the individual administered the 5-HT receptor agonist (described herein) has increased metabolism. In some embodiments, the individual administered the 5-HT receptor agonist (described herein) chooses less caloric foods.

Provided herein are methods (e.g., as described herein (e.g., administering a therapeutically effective amount of a 5-HT receptor agonist (e.g., psilocybin))) for managing or treating disorders, conditions or symptoms including but not limited to addiction disorders, such as but not limited to alcohol abuse, substance abuse, smoking, or obesity. Provided herein are methods for managing or treating disorders, conditions or symptoms including but not limited to eating disorders and auditory disorders. Provided herein are methods for managing or treating disorders, conditions or symptoms including but not limited to pain, such as but not limited to chronic pain. Provided herein are methods for managing or treating disorders, conditions or symptoms including but not limited to depression, bipolar disorder, post-traumatic stress disorder (PTSD), panic disorder, phobia, schizophrenia, psychopathy, or antisocial personality disorder. Provided herein are methods for managing or treating disorders, conditions or symptoms including but not limited to impulse disorders, such as but not limited to attention deficit hyperactivity disorder (ADHD), Tourette's syndrome or autism. Provided herein are methods for managing or treating disorders, conditions or symptoms including but not limited to compulsive disorder, such as but not limited to obsessive compulsive disorder (OCD), gambling, or aberrant sexual behavior. Provided herein are methods for managing or treating disorders, conditions or symptoms including but not limited to personality disorders, such as but not limited to conduct disorder, antisocial personality, or aggressive behavior.

Provided herein are methods (e.g., as described herein (e.g., administering a therapeutically effective amount of a 5-HT receptor agonist (e.g., psilocybin))) for managing or treating, by way of non-limiting examples:

Neurodevelopmental Disorders, such as but not limited to attention-deficit/hyperactivity disorder (ADHD), autism spectrum disorder, learning disorders and the like.

Schizophrenia Spectrum and other Psychotic Disorders, such as including but not limited to detachment from reality, delusions, hallucinations, and disorganized thinking and speech.

Bipolar and Related Disorders (e.g., which may involve episodes of mania (periods of excessive excitement, activity, and energy) alternating with periods of depression).

Depressive Disorders (e.g., which may involve feelings of extreme sadness, reduced interest in previously enjoyable activities, including but not limited to depression, severe depression, major depressive disorder (MDD), premenstrual dysphoric disorder (PMDD) and the like).

Anxiety Disorders (e.g., which may involve worrying excessively about potential bad things or situations. Examples include generalized anxiety disorder (GAD), panic disorder and phobias (irrational fears of specific things) and the like).

Obsessive-Compulsive and Related Disorders (e.g., which may involve repeated, unwanted urges, thoughts, or images (obsessions) and feeling driven to taking repeated actions in response to them (compulsions). Non-limiting examples include obsessive-compulsive disorder (OCD), hoarding disorder, extreme nail biting, and hair-pulling disorder (trichotillomania)).

Trauma, stress-related, stress-induced, and/or Stressor-Related Disorders (e.g., which may develop during or after stressful or traumatic life events. Non-limiting examples include posttraumatic stress disorder (PTSD) and acute stress disorder).

Dissociative Disorders (e.g., wherein the sense of self is may be disrupted, such as but not limited to dissociative identity disorder, dissociative amnesia and the like).

Somatic Symptom and Related Disorders (e.g., which may involve distressing and incapacitating physical symptoms with no clear medical cause. Non-limiting examples include illness anxiety disorder, somatic symptom disorder (hypochondriasis), factitious disorder and the like).

Feeding and Eating Disorders (e.g., which may involve disturbances related to eating, such as but not limited to anorexia nervosa, bulimia nervosa, and binge eating disorder).

Elimination Disorders (e.g., which may involve inappropriate elimination (release) of urine or stool by accident or deliberately, such as but not limited to bedwetting (enuresis)).

Sleep-Wake Disorders (e.g., which may involve severe sleep disorders, including but not limited to insomnia disorder, nightmare disorder, sleep apnea, and restless legs syndrome).

Disruptive, Impulse-Control, and Conduct Disorders (e.g., which may involve difficulty with emotional and/or behavioral self-control, such as but not limited to kleptomania (repeated stealing), pyromania, and intermittent explosive disorder).

Substance Related Disorders (e.g., which may involve problems associated with excessive use of substances such as alcohol (alcohol dependence, alcoholism), tobacco products, drugs, opioids (for example, cocaine, oxycodone, morphine and the like), recreational drugs, hallucinogens and the like).

Addictive Disorders (e.g., which may involve problems associated with excessive use of particular behaviors or fixations, such as but not limited to gambling disorder).

Neurocognitive Disorders (e.g., which may affect the ability to think and reason, such as but not limited to traumatic brain injury (TBI), Alzheimer's disease and the like).

Personality Disorders (e.g., which may involve enduring patterns of emotional instability and unhealthy behaviors that disrupt daily living and relationships. Examples include but are not limited to borderline, antisocial, and narcissistic personality disorders).

Gender Dysphoria (e.g., which may involve distress caused by a person's desire to be a different gender).

Sexual Dysfunctions (e.g., such as but not limited to premature ejaculation, erectile disorder, and female orgasmic disorder).

Paraphilic Disorders (sexual perversion, sexual deviation) (e.g., which may involve sexual interest in atypical objects, situations, fantasies, behaviors, or individuals. Examples include but are not limited to sexual sadism disorder, voyeuristic disorder, and pedophilic disorder).

Further examples of the disorders, conditions and symptoms which may be managed or treated (e.g., as described herein (e.g., administering a therapeutically effective amount of a 5-HT receptor agonist (e.g., psilocybin))) include by way of non-limiting examples Fragile X syndrome, Downs's syndrome, migraine headache, cluster headache, psychiatric disorders, neurodevelopmental disorders, attention-deficit/hyperactivity disorder (ADHD), autism spectrum disorder, learning disorders, schizophrenia spectrum, psychotic disorders, bipolar disorders, depression, severe depression, major depressive disorder (MDD), premenstrual dysphoric disorder (PMDD), suicidality, mood related disorders, panic disorder, panic attack, phobias, agoraphobia, selective mutism, obsessive-compulsive disorder (OCD), hoarding disorder, hair-pulling disorder (trichotillomania), excoriation (skin-picking) disorder, substance-/medication-induced obsessive-compulsive disorder, trauma related disorders, traumatic brain injury (TBI), posttraumatic stress disorder (PTSD), acute stress disorder, dissociative disorders, dissociative identity disorder, dissociative amnesia, anxiety, anxiety disorders, generalized anxiety disorder (GAD), social anxiety disorder, separation anxiety disorder, illness anxiety disorders, somatic disorders and diseases, somatic symptom disorder (hypochondriasis), factitious disorder, feeding disorders, eating disorders, anorexia, anorexia nervosa, bulimia nervosa, binge eating disorder, elimination disorders, enuresis, sleep disorders, insomnia, nightmare disorder, sleep apnea, central sleep apnea, narcolepsy, obstructive sleep apnea, hypopnea, and sleep-related hypoventilation, restless legs syndrome, jet lag, sexual dysfunction, premature ejaculation, erectile disorder, female orgasmic disorder, gender identity disorder, gender dysphoria, disruptive disorders, impulse-control disorders, conduct disorders, disruptive conduct disorders, impulse-control disorders, oppositional defiant disorder (ODD), aggression, kleptomania, pyromania, addictive disorders, substance dependence, substance abuse, alcoholism, drug addiction, opioid addiction, cocaine addiction, gambling addiction, tobacco dependence, food addiction, other forms of addiction to substances and behaviors, obesity, cognitive disorders, memory related disorders, learning related disorders, neurocognitive disorders, Alzheimer's disease, personality disorders, narcissistic personality disorders, Asperger syndrome, Tourette syndrome, Huntington's disease, Parkinson's disease, Lewy body disease, amyotrophic lateral sclerosis (ALS), Friedreich's ataxia, muscular atrophy, prion disease, dementia, vascular dementia, dementia/neurocognitive issues due to infection, dementia due to substance abuse or exposure to toxins, frontotemporal degeneration, mood disorders, delirium, aphasia, apraxia, agnosia, concussion, amnesia, anterograde amnesia, retrograde amnesia, body dysmorphic disorder, reactive attachment disorder, Fragile X syndrome, Down syndrome, migraines, migraine headache, cluster headache, cardiovascular disease, inflammatory conditions, fibromyalgia and pain.

Provided herein are methods for managing disorders or conditions, or treating symptoms of disorders or conditions, comprising administering one or more 5-HT receptor agonists, or pharmaceutically acceptable salts, solvates, metabolites, derivatives, or prodrugs thereof. In some embodiments, the (e.g., one or more) 5-HT receptor agonist is a 5-HT2 receptor agonist. In some embodiments, the 5-HT2 receptor agonist is a 5-HT_{2A} receptor agonist, a 5-HT_{2B} receptor agonist and/or a 5-HT_{2C} receptor agonist. In some embodiments, the (e.g., one or more) 5-HT receptor agonist is psilocin or psilocybin or a pharmaceutically acceptable salt, solvate, metabolite, derivative, or prodrug thereof. In some embodiments, the therapeutically effective amount of the 5-HT receptor agonist or a pharmaceutically acceptable salt, solvate, metabolite, derivative, or prodrug thereof is provided to the subject in need thereof in an amount insufficient to provide a hallucinogenic experience

In some embodiments, the therapeutically effective amount of 5-HT receptor agonist or a pharmaceutically acceptable salt, solvate, metabolite, derivative, or prodrug thereof is provided to a subject in need thereof in an amount and/or formulation insufficient to provide a maximum plasma concentration (Cₘₐₓ) of (e.g., active form of the) 5-HT receptor agonist or a pharmaceutically acceptable salt, solvate, metabolite, derivative, or prodrug thereof of 6 ng/mL or more. In some embodiments, the therapeutically effective amount of 5-HT receptor agonist or a pharmaceutically acceptable salt, solvate, metabolite, derivative, or prodrug thereof is provided to a subject in need thereof in an amount and/or formulation to provide a maximum plasma concentration (Cₘₐₓ) of (e.g., active form of the) 5-HT receptor agonist or a pharmaceutically acceptable salt, solvate, metabolite, derivative, or prodrug thereof of about 0.1 ng/mL or more and less than 6 ng/mL (e.g., at least 0.5 ng/mL and less than 6 ng/mL, about 1 ng/mL to about 5.5 ng/mL, about 2 ng/mL to about 5 ng/mL, or the like). In some embodiments, the therapeutically effective amount of 5-HT receptor agonist or a pharmaceutically acceptable salt, solvate, metabolite, derivative, or prodrug thereof is provided to a subject in need thereof in an amount and/or formulation to provide a plasma concentration of (e.g., active form of the) 5-HT receptor agonist or a pharmaceutically acceptable salt, solvate, metabolite, derivative, or prodrug thereof of at least 0.1 ng/mL (e.g., at least 0.2 ng/mL, at least 0.3 ng/mL, at least 0.5 ng/mL, or the like) after at least 6 hours (e.g., at least 12 hours, at least 24 hours, at least 36 hours, at least 48 hours, at least 72 hours, at least 96 hours, at least 120 hours, at least 144 hours, or the like).

In some embodiments, the pharmaceutical composition is an oral formulation, a buccal formulation, a nasal formulation, or an inhalation formulation. In some embodiments, the pharmaceutical composition is in a form selected from a spray, aerosol, mist, nebulae, ointment, cream, gel, paste, salve, solution, suspension, tincture, patch, and atomized vapor.

### Therapeutic Regimens

In some embodiments, any pharmaceutical composition or formulation or 5-HT receptor agonist agent disclosed herein is administered for therapeutic application. In some embodiments, the pharmaceutical composition or formation or 5-HT receptor agonist agent is administered once per day, twice per day, three times per day or more. In certain embodiments, the pharmaceutical composition or formulation or 5-HT receptor agonist agent is administered daily, every day, every alternate day, five days a week, once a week, every other week, two weeks per month, three weeks per month, once a month, twice a month, three times per month, or more. In some embodiments, the pharmaceutical composition or formulation or 5-HT receptor agonist agent is administered for at least 1 month, 2 months, 3 months, 4 months, 5 months, 6 months, 7 months, 8 months, 9 months, 10 months, 11 months, 12 months, 18 months, 2 years, 3 years, or more.

In some embodiments, one or more pharmaceutical compositions are administered simultaneously, sequentially, or at an interval period of time. In some embodiments, one or more pharmaceutical compositions are administered simultaneously. In some cases, one or more pharmaceutical compositions are administered sequentially. In additional cases, one or more pharmaceutical compositions are administered at an interval period of time (e.g., the first administration of a first pharmaceutical composition is on day one followed by an interval of at least 1, 2, 3, 4, 5, or more days prior to the administration of at least a second pharmaceutical composition).

In some embodiments, two or more different pharmaceutical compositions are co-administered. In some instances, the two or more different pharmaceutical compositions are co-administered simultaneously. In some cases, the two or more different pharmaceutical compositions are co-administered sequentially without a gap of time between administrations. In other cases, the two or more different pharmaceutical compositions are co-administered sequentially with a gap of about 0.5 hour, 1 hour, 2 hour, 3 hour, 12 hours, 1 day, 2 days, or more between administrations.

In some embodiments, the amount of a given agent that corresponds to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages is altered depending on a number of variables, not limited to the activity of the compound used, the disease or condition to be treated, the mode of administration, the requirements of the individual subject, the severity of the disease or condition being treated, and the judgment of the practitioner.

### EXAMPLES

The examples are provided for illustrative purposes only and not to limit the scope of the claims provided herein.

### Example 1: Psilocybin Dose and Efficacy Studies (e.g., Immediate Release Study)

### Dose Finding Experiments

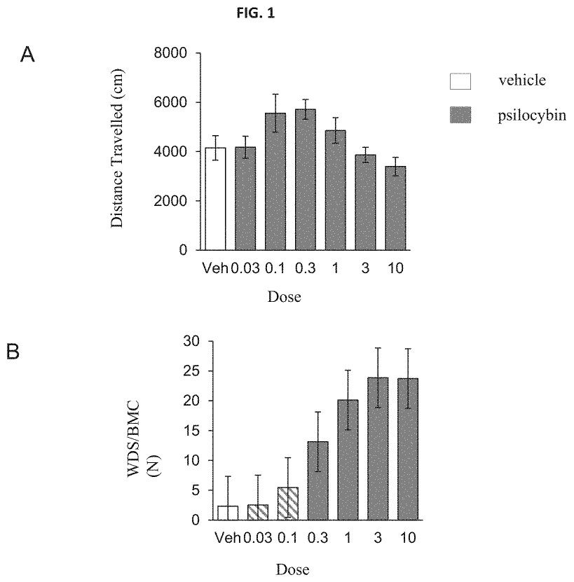

Psilocybin was tested at a dose range of 0.03-10.0 mg/kg administered subcutaneously (S.C.) Behavioral syndrome including wet dog shakes (WDS) and back muscle contractions (BMC) are characteristic of 5-HT_{2A} receptor activity and potentially indicative of psychomotor signs. Psilocybin dosed 0.03-0.1 mg/kg S.C. produced no signs of behavioral syndrome, whereas doses of 0.3 mg/kg I.P and higher produced significant signs of behavior syndrome (e.g., FIG. 1B (WDS/BMC)). These results suggest that a dose range of 0.03-0.1 mg/kg S.C. is preferred for examining precognitive or motivational-enhancing effects of psilocybin. There is a parallel between behaviors such as WDS in rats and hallucination in humans (Behavioral Neurobiology of Psychedelic Drugs, Halberstadt, Adam, Vollenweider, Franz X., Nichols, David E. (Eds.), Springer, 2018, p 161).

Compared with stimulant drugs, low doses of psilocybin (light bar) produce an increase in motor activity distance travelled at low doses (FIG. 1A) and high doses (dark bar) showing slightly decreased distanced travelled. Stimulant drugs cause far larger increases in distance travelled with escalating doses, reflective of a direct motor stimulant property not seen with psilocybin.

### Efficacy Studies

General: Experiments were conducted to test psilocybin doses in the range of 0.01-0.2 mg/kg psilocybin administered S.C. across progressive ratio (PR) and 5-choice serial reaction time task (5CSRTT) to examine the effect of psilocybin on endophenotypes of motivation and attention. In each study, a population of out-bred Long Evans rats was first tested as a group for response to the PR and 5CSRTT. Each group was then divided into subgroups by tertiles according to performance, and then exposed to various dose levels immediately prior to re-testing at various psilocybin dose levels. As explained below, rats in the lowest tertile sub-group displayed different responses than rats in the highest tertile.

The PR test is used to answer how willing the test subject is to work for food (i.e. test of motivation). A single 45 mg food pellet (i.e. reinforcer) is made available to the test animal based on lever press response. To obtain each successive pellet, the animal must make increasingly more lever presses. Typically, a progression of 2, 4, 6, 9, 12, 15, 20, 25, 32, 40, 50, 62, 77, 95, 118, etc. is used, derived from the equation:

ratio=[5×e^{(0.2×reinforcer #)}−5]

Hungry test animals do not find 45 mg food pellets sufficient and, therefore, there is a drive to repeatedly lever press to obtain multiple food pellets. At some point, the animal gives up as the motor demands to obtain a single pellet are not deemed worthwhile (i.e. animal reaches “break point” defined as animal's failure to earn a food pellet in 20 minutes).

Study A: Psilocybin was administered to rats S.C. at doses of 0.05, 0.1 and 0.2 mg/kg. None of these doses were observed to cause obvious changes in the number of lever presses or break points (i.e. rewards earned) when studied across entire study population of 36 rats.

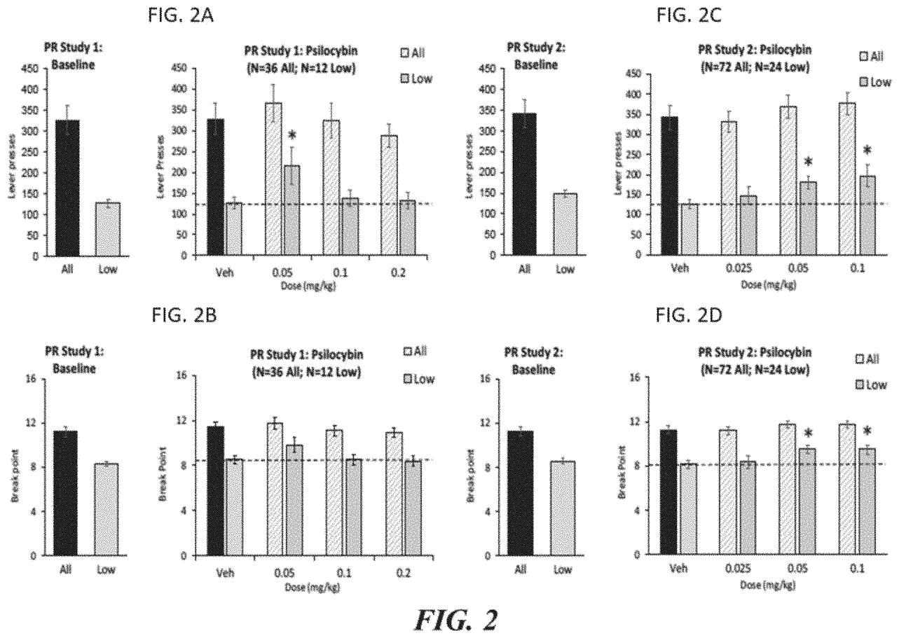

Rats were then sub-grouped into tertiles based on the number of lever presses for food at baseline, so that within the test population of 36 animals, 12 animals were identified as low responders, characterized by low motivation and potentially corresponding to low motivation endophenotype representative of clinical depression. Twelve animals were identified as high responders. There was a statistically significant difference between the high and low responders. Psilocybin doses of 0.05-0.2 mg/kg administered S.C. were observed to produce no effect on response in high responder subgroup (FIG. 2A and FIG. 2B). However, 0.05 mg/kg psilocybin was observed to produce an increase in food responding in the low responder subgroup, whereas the dose of 0.1 mg/kg did not produce an increase in food responding. At the 0.05 mg/kg dose, there was a significant increase in number of lever presses and break point (P<0.05 vs. vehicle, paired T-test), and a trend to increased session duration (Veh: 7.8+1.1 min, psilocybin 0.05 mg/kg: 10.8+1.9 min; P=0.07 vs. vehicle, paired T-test). This represents a motivational enhancing effect of psilocybin at the 0.05 mg/kg dose in the cohort corresponding to a low motivation endophenotype representative of clinical depression.

Study B: Based on Study 1 outcome, a follow-up PR study was conducted utilizing a larger cohort size (72 rats total), and incorporating a lower dose of psilocybin (0.025 mg/kg SC) (FIG. 2C and FIG. 2D). Similar to Study 1, assessed over all 72 rats, there was no main effect of treatment on number of lever presses (F3,210=1.9, P=0.1; NS), or session duration (F3,210=1.2, NS). A main effect of treatment was recorded for break point (F3,210=3.3; P=0.02), however no treatment group was significantly different to vehicle. Separation of rats based on the lowest average break point recorded over the 7 days prior to testing identified a “low responder” subgroup of N=24 rats. Analysis confined to this “low responder” cohort revealed significant effects of treatment on lever press (F3,69=4.9, P<0.01) and break point (F3,69=5.6; P<0.01), reflecting increases in both measures following psilocybin 0.05-0.1 mg/kg pretreatment relative to vehicle. There was no treatment effect on session duration (F3,69=2.4; P=0.07, NS), although there was trend for this to be increased at the 0.1 mg/kg dose (Veh: 15.1+2.0 min; Psilo 0.1 mg/kg: 18.7+2.5 min). In addition, no significant effect was observed in rats treated with the lowest dose of LSD tested (e.g., 0.025 mg/kg), showing that there is a lower bound (e.g., a therapeutically effective threshold below the hallucinogenic threshold) in that it shows a low dose that does not have beneficial effects.

The 5CSRTT involves evaluating test subject response to a brief visual stimulus (Higgins, Guy & Silenieks, Leonardo. (2017). Rodent Test of Attention and Impulsivity: The 5-Choice Serial Reaction Time Task: The 5-Choice Serial Reaction Time Task. 10.1002/cpph.27). Animals are trained to make a nose-poke response to a stimulus location in order to collect a food reward. The task allows the experimenter to measure animal performance in multiple domains including:
Attention
Impulsivity
Perseveration
Speed of response

A strength of the test is its flexible configuration to challenge test subjects:
Standard test conditions (0.75 s stimulus duration (SD), 5 s inter-trial interval (ITI), 100 trials)
Multiple short stimulus duration (mSD) (0.03-1 s SD)
Fixed long ITI (5 s vs. 10 s ITI)
Multiple ultrashort ITI (2-5 s ITI)
Extended 250 trials

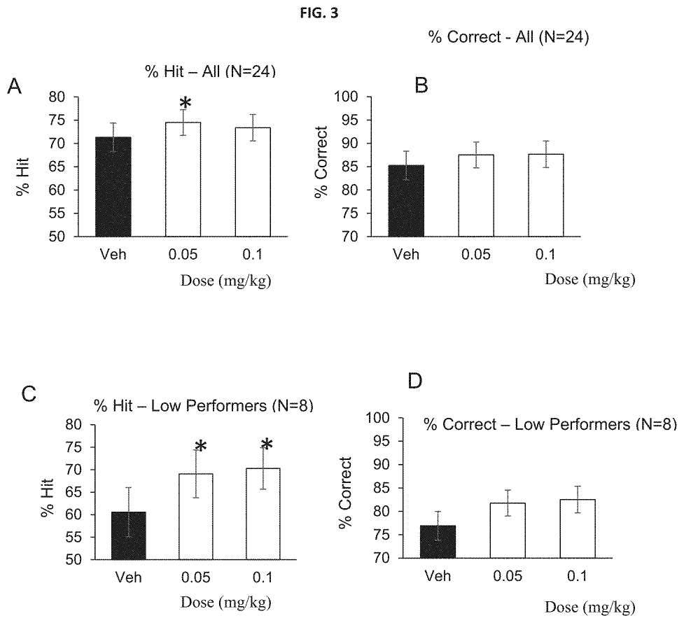

Psilocybin dose of 0.05 mg/kg administered S.C. was observed to produce an increased (p=0.05, t-test) pro-cognitive effect (measured as % hit, calculated as # correct/(# correct+# incorrect+# omissions)*100) when studied across a study population of 24 rats in a 5CSRTT with standard conditions, i.e. 0.75 s SD, SS ITI, 100 trials. Asterisk (*) indicates statistical significance vs. vehicle. (FIG. 3A). There was a slight non-significant increase in procognitive effect (measured as % correct, calculated as # correct/(# correct+# incorrect)*100) (FIG. 3B). For the low performers, psilocybin dose of both 0.05 mg/kg and 0.1 mg/kg administered S.C. was observed to produce an increased (p=0.05, t-test) pro-cognitive effect (measured as % hit, calculated as # correct/(# correct+# incorrect+# omissions)*100) (FIG. 3C), while a slight non-significant increase in pro-cognitive effect measured as % correct (calculated as # correct/(# correct+# incorrect)*100) was observed (FIG. 3D). There was no effect on performance, e.g. no effect on response speed, or number of trials completed. There was no effect on premature or perseverative responses in this experiment.

When this population is segmented into tertiles according to performance based on accuracy (% correct, calculated as # correct/(# correct+# incorrect)*100), the lowest performing tertile (N=8) are considered to be low attentive and potentially representative of a low attentive endophenotype of depression (FIG. 6A). Low attentive rats also score poorly on % hit (FIG. 6B) and have a slower response speed (FIG. 6D). Similar to the PR test, the effect of 0.05 and 0.1 mg/kg psilocybin on accuracy (% correct and % hit) in the 5CSRTT was observed to be strongly evident in the low attentive subgroup compared with vehicle (FIG. 6C and FIG. 6E). Asterisk (*) indicates statistical significance vs. vehicle. Psilocybin 0.05 mg/kg also increased response speed in the low attentive cohort compared with vehicle (FIG. 6D).

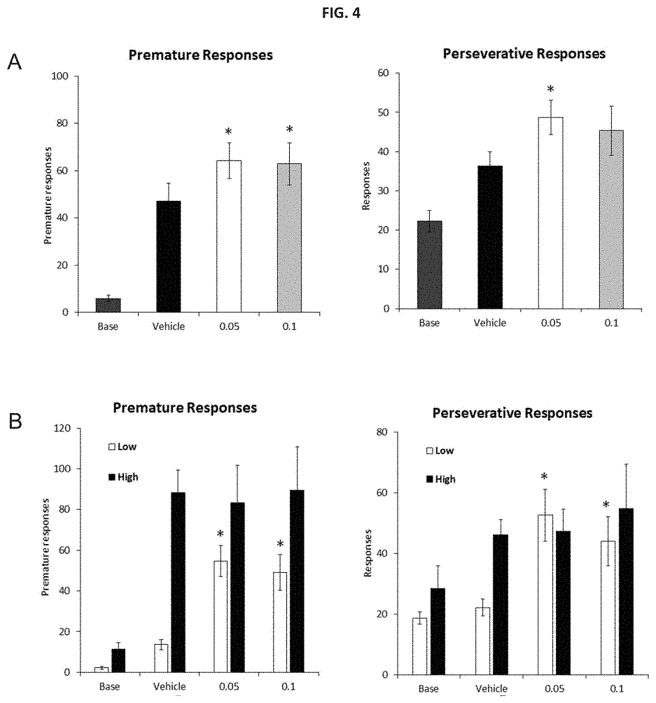

Using a longer duration between stimulus and reward, the 5CSRTT study measures premature (PREM) and perseverant (PSV) responses. PREM/PSV responses were increased by increasing the ITI from 5 s (baseline) to 10 s (test condition). A psilocybin dose of 0.05 mg/kg administered S.C. was observed to produce an increase (p=0.05, t-test) increase in PREM and PSV responses under a 10 s ITI when studied across a study population of 24 rats (FIG. 4A). Low responders (N=8) were observed to improve significantly from psilocybin administration at both 0.05 and 0.1 mg/kg doses (FIG. 4B, p<0.01, t-test), with marked increases in premature responses and perseverative responses in rats in that sub-group compared with vehicle. Both PREM and PSV behaviors are examples of executive cognitive function, likely involving areas of the prefrontal cortex, a brain region rich in 5-HT_{2A} receptors.

At doses that did not produce behavioral effects in animals indicative of hallucination in humans, improved results were seen on low performing animals on measures of motivation, attention, accuracy, speed of response, perseveration, and cognitive engagement. The improvement in the low performing animals indicates utility of non-hallucinogenic doses of psilocybin in treatment of behavioral and cognitive disorders involving these behaviors, including but not limited to depression, anxiety, apathy and low motivation, attention disorders, disorders of executive function and cognitive engagement, obsessive compulsive disorder, and neurocognitive disorders. At these same doses, no detrimental effects of psilocybin were noted on performance, e.g., there was no evidence of reduced motivation, impaired motor control, or impaired attention or response speed. The positive effects of the low doses of psilocybin appear most evident in the low performer subgroups based on four tests: one PR (motivation) and two 5CSRTT (attention) studies were conducted, using psilocybin at 0.05-0.2 mg/kg and 0.025-0.1 mg/kg (PR) and 0.05-0.1 mg/kg S.C. (5CSRTT). Significant improvements were noted in the low performing animals on:
Number of lever presses and increased break point in PR test (0.05 mg/kg and 0.1 mg/kg)
% Correct and % Hit in 5CSRTT (0.05 and 0.1 mg/kg)
Increased speed of responding in 5CSRTT (0.05 mg/kg)
PREM/PSV in 5CSRTT 10 s ITI (0.05 and 0.1 mg/kg)

### Example 2: Psilocybin and Psilocin Pharmacokinetics (PK)

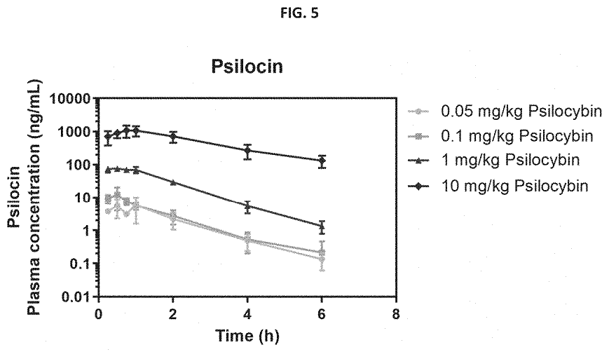

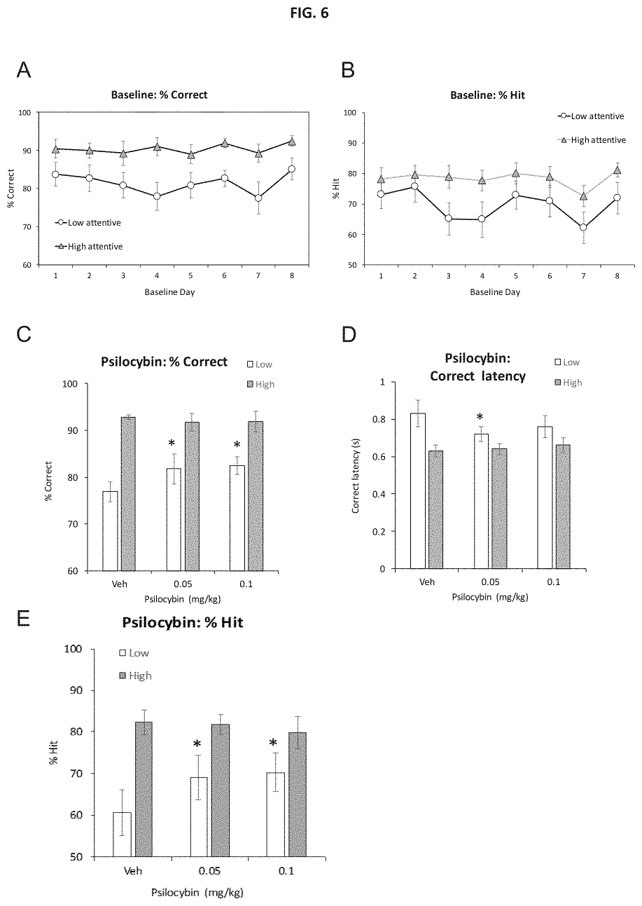

Psilocybin doses of 0.05, 0.1, 1, 10 mg/kg were used to evaluate psilocybin and psilocin PK in rats. For the doses of psilocybin that positively effect behaviors in the low performers in the PR and 5CSRTT, e.g., 0.05-0.1 mg/kg, the corresponding Cm. of psilocin was ˜7±2 ng/ml at 30 minutes for 0.05 mg/kg, and at a dose of 0.1 mg/kg, the Cm. of psilocin was determined to be ˜12±3 ng/ml at 30 minutes (FIG. 5).

Details of the plasma concentration studies are shown in Tables 1-8 (psilocybin) and 9-16 (psilocin). Values in italics are below the lower level of quantitation (BLQ, <1 ng/mL) but were included in calculations. Values in bold and underlined are considered to be outliers and were omitted from calculations. Measured dosing solution concentrations were 0.0460, 0.0967, 0.948 and 9.65 mg/mL for 0.05, 0.1, 1 and 10 mg/mL respectively.

TABLE 1
Plasma concentrations of psilocybin following
0.05 mg/kg s.c. administration

| | Plasma concentration | |
|---|---|---|
| Experimental time | (ng/mL) | |
| (h) | Mean | SD |
| 0.25 | 6.20 | 7.27 |
| 0.5 | 12.5 | 16.9 |
| 0.75 | 1.07 | 0.348 |
| 1 | 19.2 | 26.3 |
| 2 | 0.594 | — |
| 4 | 1.95 | 0.508 |
| 6 | 2.12 | — |

TABLE 2
Plasma concentration of psilocybin following
0.1 mg/kg s.c. administration

| | Plasma | |
|---|---|---|
| | concentration | |
| Experimental time | (ng/mL) | |
| (h) | Mean | SD |
| 0.25 | 21.7 | 27.4 |
| 0.5 | 28.9 | 45.0 |
| 0.75 | 2.96 | 1.32 |
| 1 | 2.19 | 0.605 |
| 2 | 1.76 | |
| 4 | 2.55 | |
| 6 | 0.921 | |

TABLE 3
Plasma concentration of psilocybin following
1.0 mg/kg s.c. administration

| | Plasma | |
|---|---|---|
| | concentration | |
| Experimental time | (ng/mL) | |
| (h) | Mean | SD |
| 0.25 | 75.2 | 39.8 |
| 0.5 | 47.3 | 24.4 |
| 0.75 | 35.8 | 13.2 |
| 1 | 35.1 | 30.4 |
| 2 | 2.19 | 1.26 |
| 4 | 1.12 | — |
| 6 | 0.929 | — |

TABLE 4
Plasma concentration of psilocybin following
10 mg/kg s.c. administration

| | Plasma | |
|---|---|---|
| | concentration | |
| | (ng/mL) | |
| Experimental time (h) | Mean | SD |
| 0.25 | 1030 | 832 |
| 0.5 | 1544 | 1898 |
| 0.75 | 344 | 132 |
| 1 | 269 | 93.4 |
| 2 | 101 | 172 |
| 4 | 1.80 | 0.985 |
| 6 | 1.32 | 1.02 |

TABLE 5
Plasma PK parameters summary for psilocybin following 0.05 mg/kg s.c.
administration.

| Parameter | Mean | SD |
|---|---|---|
| tₘₐₓ (h) | 0.600 | 0.379 |
| Cₘₐₓ (ng/mL) | 18.9 | 20.9 |
| Cₘₐₓ/Dose (kg*ng/mL/mg) | 377 | 419 |
| Apparent t_{1/2} (h) | — | — |
| AUC₀₋ₜₗₐₛₜ (h*ng/mL) | 10.7 | 7.08 |
| AUC_{0-inf} (h*ng/mL) | — | — |
| AUC_{0-inf}/Dose (h*kg*ng/mL/mg) | — | — |
| MRT_{0-inf} (h) | — | — |

Tₘₐₓ = time at which maximum concentration is observed
Cₘₐₓ = maximum observed concentration
Apparent t_{1/2} = apparent terminal half-life
AUC₀₋ₜₗₐₛₜ = Area Under the Concentration vs time curve from time 0 to the time of the last measurable concentration
AUC_{0-inf} = Area Under the Concentration vs time curve from time to infinity
MRT_{0-inf} = Mean Residence Time from time zero to infinity

TABLE 6
Plasma PK parameters summary for psilocybin following
0.1 mg/kg s.c. administration.

| Parameter | Mean | SD |
|---|---|---|
| tₘₐₓ (h) | 0.321 | 0.122 |
| Cₘₐₓ (ng/mL) | 31.4 | 39.9 |
| Cₘₐₓ/Dose (kg*ng/mL/mg) | 314 | 399 |
| Apparent t_{1/2} (h) | — | — |
| AUC₀₋ₜₗₐₛₜ (h*ng/mL) | 11.4 | 11.9 |
| AUC_{0-inf} (h*ng/mL) | — | — |
| AUC_{0-inf}/Dose (h*kg*ng/mL/mg) | — | — |
| MRT_{0-inf} (h) | — | — |

TABLE 7
Plasma PK parameters summary for psilocybin following 1.0 mg/kg s.c.
administration.

| Parameter | Mean | SD |
|---|---|---|
| tₘₐₓ (h) | 0.357 | 0.283 |
| Cₘₐₓ (ng/mL) | 78.3 | 39.8 |
| Cₘₐₓ/Dose (kg*ng/mL/mg) | 78.3 | 39.8 |
| Apparent t_{1/2} (h) | 0.445 | 0.236 |
| AUC₀₋ₜₗₐₛₜ(h*ng/mL) | 53.1 | 23.7 |
| AUC_{0-inf} (h*ng/mL) | 47.9 | 17.6 |
| AUC_{0-inf}/Dose (h*kg*ng/mL/mg) | 47.9 | 17.6 |
| MRT_{0-inf} (h) | 0.746 | 0.186 |

TABLE 8
Plasma PK parameters summary for psilocybin
following 10 mg/kg s.c. administration

| Parameter | Mean | SD |
|---|---|---|
| tₘₐₓ (h) | 0.500 | 0.289 |
| Cₘₐₓ (ng/mL) | 3595 | 4663 |
| Cₘₐₓ/Dose (kg*ng/mL/mg) | 359 | 466 |
| Apparent t_{1/2} (h) | 0.566 | 0.189 |
| AUC₀₋ₜₗₐₛₜ(h*ng/mL) | 1707 | 2338 |
| AUC_{0-inf} (h*ng/mL) | 2156 | 2704 |
| AUC_{0-inf}/Dose (h*kg*ng/mL/mg) | 216 | 270 |
| MRT_{0-inf} (h) | 0.716 | 0.175 |

TABLE 9
Plasma concentrations of psilocin following 0.05 mg/kg s.c.
administration of psilocybin.

| | Plasma concentration | |
|---|---|---|
| Experimental time | (ng/mL) | |
| (h) | Mean | SD |
| 0.25 | 3.76 | 0.560 |
| 0.5 | 5.68 | 3.38 |
| 0.75 | 3.11 | 0.245 |
| 1 | 5.91 | 4.31 |
| 2 | 2.23 | 1.18 |
| 4 | 0.490 | 0.253 |
| 6 | 0.136 | 0.0749 |

TABLE 10
Plasma concentrations of psilocin following 0.1 mg/kg s.c.
administration of psilocybin.

| | Plasma concentration | |
|---|---|---|
| Experimental time | (ng/mL) | |
| (h) | Mean | SD |
| 0.25 | 9.35 | 2.79 |
| 0.5 | 12.3 | 8.29 |
| 0.75 | 7.73 | 1.87 |
| 1 | 5.70 | 1.52 |
| 2 | 2.78 | — |
| 4 | 0.535 | — |
| 6 | 0.215 | — |

TABLE 11
Plasma concentrations of psilocin following 1 mg/kg s.c.
administration of psilocybin.

| | Plasma concentration | |
|---|---|---|
| Experimental time | (ng/mL) | |
| (h) | Mean | SD |
| 0.25 | 74.5 | 16.7 |
| 0.5 | 76.6 | 11.1 |
| 0.75 | 70.3 | 7.05 |
| 1 | 69.9 | 15.5 |
| 2 | 29.7 | 4.76 |
| 4 | 5.50 | 2.21 |
| 6 | 1.34 | 0.551 |

TABLE 12
Plasma concentrations of psilocin following 10 mg/kg s.c.
administration of psilocybin.

| | Plasma concentration | |
|---|---|---|
| Experimental time | (ng/mL) | |
| (h) | Mean | SD |
| 0.25 | 704 | 327 |
| 0.5 | 898 | 266 |
| 0.75 | 1081 | 437 |
| 1 | 1067 | 367 |
| 2 | 713 | 255 |
| 4 | 269 | 126 |
| 6 | 132 | 53.3 |

TABLE 13
Plasma PK parameters summary for psilocin following 0.05 mg/kg
s.c. administration of psilocybin.

| Parameter | Mean | SD |
|---|---|---|
| tₘₐₓ (h) | 0.600 | 0.379 |
| Cₘₐₓ (ng/mL) | 7.14 | 4.02 |
| Apparent t_{1/2} (h) | 1.00 | 0.227 |
| AUC₀₋ₜₗₐₛₜ (h*ng/mL) | 10.1 | 3.96 |
| AUC_{0-inf} (h*ng/mL) | 10.3 | 3.92 |
| MRT_{0-inf} (h) | 1.73 | 0.177 |

TABLE 14
Plasma PK parameters summary for psilocin following 0.1 mg/kg
s.c. administration of psilocybin.

| Parameter | Mean | SD |
|---|---|---|
| tₘₐₓ (h) | 0.536 | 0.267 |
| Cₘₐₓ (ng/mL) | 11.7 | 6.81 |
| Apparent t_{1/2} (h) | 0.918 | 0.22 |
| AUC₀₋ₜₗₐₛₜ (h*ng/mL) | 12.5 | 6.42 |
| AUC_{0-inf} (h*ng/mL) | 15.7 | 5.56 |
| MRT_{0-inf} (h) | 1.42 | 0.337 |

TABLE 15
Plasma PK parameters summary for psilocin following 1 mg/kg
s.c. administration of psilocybin.

| Parameter | Mean | SD |
|---|---|---|
| tₘₐₓ (h) | 0.571 | 0.313 |
| Cₘₐₓ (ng/mL) | 83.3 | 14.4 |
| Apparent t_{1/2} (h) | 0.878 | 0.0762 |
| AUC₀₋ₜₗₐₛₜ (h*ng/mL) | 117 | 45.2 |
| AUC_{0-inf} (h*ng/mL) | 143 | 20.4 |
| MRT_{0-inf} (h) | 1.53 | 0.110 |

TABLE 16
Plasma PK parameters summary for psilocin following 10 mg/kg
s.c. administration of psilocybin.

| Parameter | Mean | SD |
|---|---|---|
| tₘₐₓ (h) | 0.714 | 0.267 |
| Cₘₐₓ (ng/mL) | 1106 | 434 |
| Apparent t_{1/2} (h) | 1.65 | 0.325 |
| AUC₀₋ₜₗₐₛₜ (h*ng/mL) | 2280 | 1524 |
| AUC_{0-inf} (h*ng/mL) | 3376 | 1056 |
| MRT_{0-inf} (h) | 2.66 | 0.395 |

### Example 3. Treatment of Low-Motivation in Rats with Extended Release of Psilocybin General

The 5-choice serial reaction time task (5-CSRTT) was used to measure attentional performance in rodents. The test design involves training animals to respond to a brief visual stimulus presented unpredictably in one of five locations. Once trained to stable performance levels, the effects of experimental manipulations on response speed and choice accuracy were measured and each are related to attentional performance. The test was used to examine aspects of response control with responding prior to stimulus onset, i.e., a premature response, becoming a measure of motor impulsivity. The test is adaptable to task modification. Variations to stimulus duration (SD, 0.03-1 s duration) and frequency of stimulus presentation (2-10 s), amongst others have become commonly used to tax performance.

### Pre-Training

72 male Long Evans rats (source: Charles River, St. Constant, QE) were trained to asymptotic performance in the 5-CSRTT over a period of approximately 2 months. The rats were trained to final stimulus duration (SD) of 0.75 s, 5 s inter-trial interval (ITI), 5 s limited hold (LH), 100 trials per session. The rats were singly housed and food restricted to ˜20 g food/day throughout the study.

While the target performance levels under these conditions was in the range of >80% accuracy, <20% omissions, individual rats show different performance levels based on attentional accuracy or measures of impulsive action. The selection phase involves selecting the “worst” 40 rats based on both attentional performance (lowest performance based on accuracy measures of % correct/% hit) and/or impulsive action (lowest levels of premature responses noted at an extended 10 s ITI schedule and also baseline testing).

A dose of psilocybin, which is free from perceptual and behavioral side effects, was dosed in rats (N=24) continuously over a period of 12 days using an Alzet minipump implanted in rats of approximately 380-390 g body weight. The pump was primed to deliver psilocybin at 5 mg/kg/day pump concentration. The target plasma concentration was 10 ng/ml psilocin with actual plasma concentration 9.3+1.3 ng/ml over the 12 day study period.

There were no changes in motor activity (distance travelled, rearing counts), or induction of overt stereotypies, including cardinal signs of 5-HT_{2A} receptor activation (Wet Dog Shakes and Back Muscle Contractions (WDS, BMD). At the completion of 12 days testing the rats were euthanized and cardiac and brain tissue were collected and snap frozen. Histological analysis was performed for the samples.

### Test Study

During the training period, 72 rats were trained on the 5-CSRTT, and the 40 worst performers. The 40 animals with the “worst” attentional performance (lowest performance based on accuracy measures of % correct, omissions and response speed) and impulsive action (PREM responses) were selected for the experiment. These 40 poor performing rats were allocated into one of two cohorts, with N=16 rats implanted with a vehicle (saline, Group A) minipumps and N=24 implanted with minipumps primed with psilocybin.

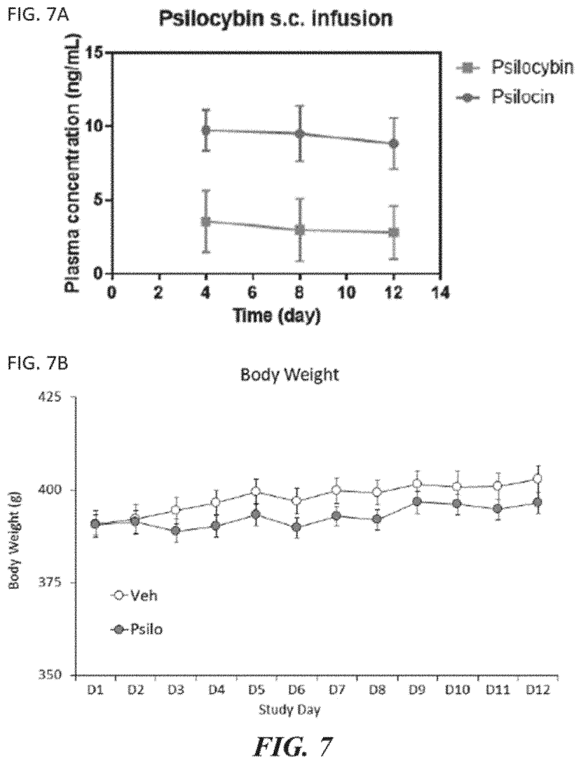

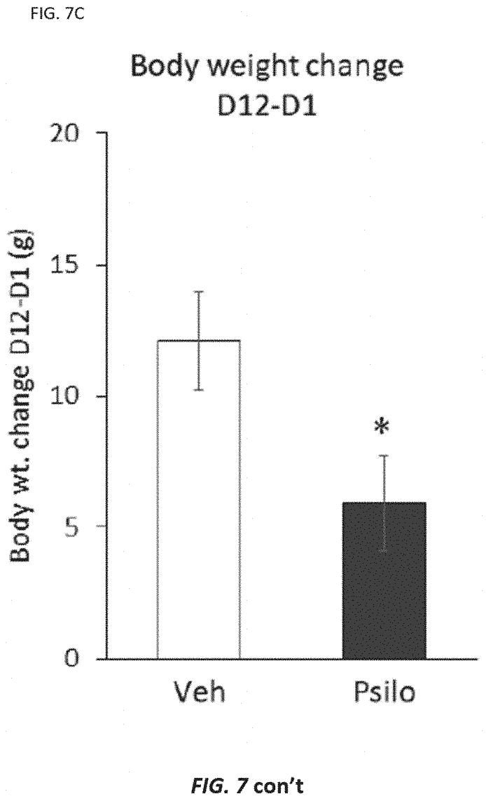

Blood was collected from rats on days 4, 8 and 12 to measure levels of psilocybin and psilocin in Group B rats. Blood was collected via saphenous bleed at a standard timepoint which will not interfere with 5-choice testing. Psilocin blood levels were in the range of 8-10 ng/ml over days 4-12, while psilocybin blood levels were in the range of 2-5 ng/ml over days 4-12 (FIG. 7A), corresponding to 10 ng/mL psilocin. Desired target exposure of psilocin was achieved for the duration of the study. The present observations recorded for psilocybin/psilocin are occurring at exposure levels of psilocin ˜10 ng/ml (e.g., FIG. 5), likely resulting in a low level of CNS 5-HT_{2A} receptor occupancy, and which is insufficient to elicit cardinal signs of 5-HT_{2A} receptor activation such as WDS/BMC in rats (e.g., FIG. 1B). Furthermore, all rats appeared healthy over the study course, with both placebo and psilocybin rats gaining body weight over the 12 day study course (FIG. 7B).

Table 17. Summary of the weight gain for both the placebo and psilocybin rats over the course of the study.

As shown in Table 17, and further illustrated in FIG. 7C, both placebo and psilocybin treated rats were healthy and gained weight. However, the psilocybin treated animals gained weight to a lesser amount that the placebo treated animals, indicating that psilocybin is effective in the management of weight gain.

TABLE 17

| | | Body Weight | | |
|---|---|---|---|---|
| | | | | Day 1 vs. Day |
| Treatment | | Day 1 | Day 12 | 12 |
| Vehicle | Average | 395.0 | 419.4 | 24.4 |
| | SEM | 9.7 | 8.9 | 5.0 |
| Psilocybin | Average | 396.8 | 417.3 | 20.6 |
| (4 mg/mL) | SEM | 7.8 | 7.0 | 3.3 |
| Psilocybin | Average | 403.5 | 422.8 | 19.3 |
| (6 mg/mL) | SEM | 7.3 | 8.9 | 2.3 |
| Psilocybin | Average | 397.8 | 422.2 | 24.4 |
| (8 mg/mL) | SEM | 9.8 | 8.4 | 5.1 |

For 12 days following pump implantation surgery, rats were assessed for performance in the 5-CSRTT under (A) standard 5-CSRTT protocol (5 s ITI, 0.75 s SD, 100 trials) on days 1-4, 6-8, 10-11 (i.e. 9 days total run under this condition), and (B) the more difficult conditions of 10 s ITI, 0.3 s SD, 100 trials. The standard conditions were tested on days 1-4, 6-8, 10-11, and the more difficult conditions were tested on post-implant days 5, 9 and 12 (i.e. 3 days total run under this condition).

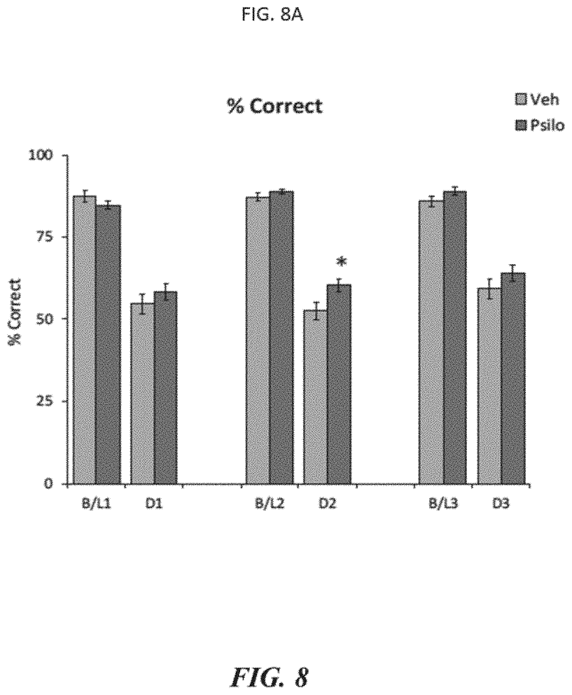

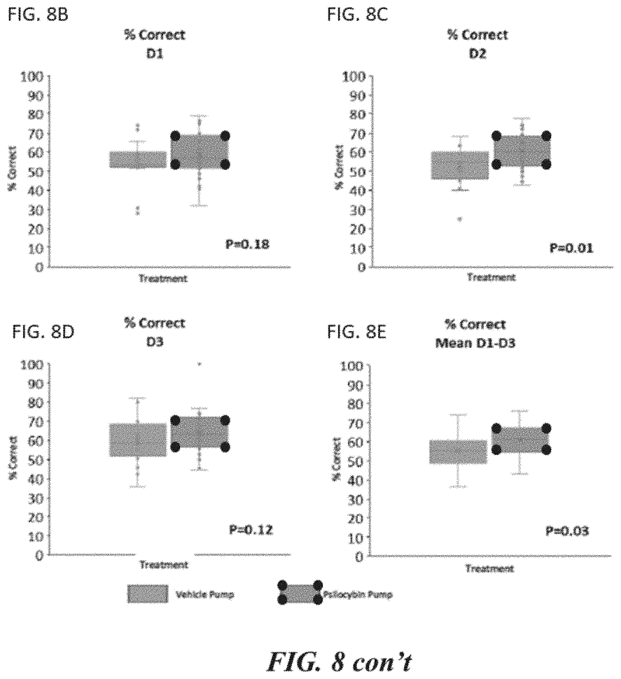

Under the more challenging conditions imposed on test days 5, 9, and 12 (D1, D2 and D3 of FIG. 8A), the psilocybin treated animals performed better than vehicle treated animals on % correct responses. The result was significant on D2 (P<0.05). In FIG. 8A, B/L refers to ‘baseline’ conditions of 5 s ITI, 0.75 SD (calculated by taking the mean of the days immediately prior to and immediately following the test day). For example, B/L1 is the mean of performance on the days before and after D1 (since D1 is test day 5, B/L is mean of days 4 and 6). Comparison of the mean 10 s ITI values revealed a significant (P=0.01) increase in % correct in the psilocybin treated group on Challenge Day 2 (p=0.01) and the mean performance on Days 1-3 was also significantly improved vs. vehicle (p=0.03) (FIG. 8B-FIG. 8E).

### Example 4. Treatment of Low-Motivation in Rats with a Combination of Immediate and Extended Release of Psilocybin

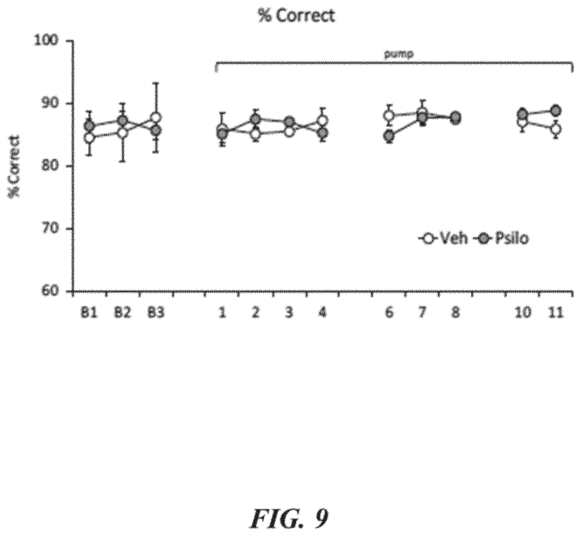

The results in the minipump study did not show that psilocybin caused an increase in correct attempts during the standard test conditions over a period of time (FIG. 9). However, as shown in FIG. 6 (e.g., FIG. 6A and FIG. 6C), acutely treated animals showed an improvement in % correct during standard test conditions. Furthermore, the minipump (e.g., ER-dosing) did cause an improvement in % correct over time. So, a combination of immediate release dosing as well as a controlled release dosing has a preferred effect on attention and cognition over time.

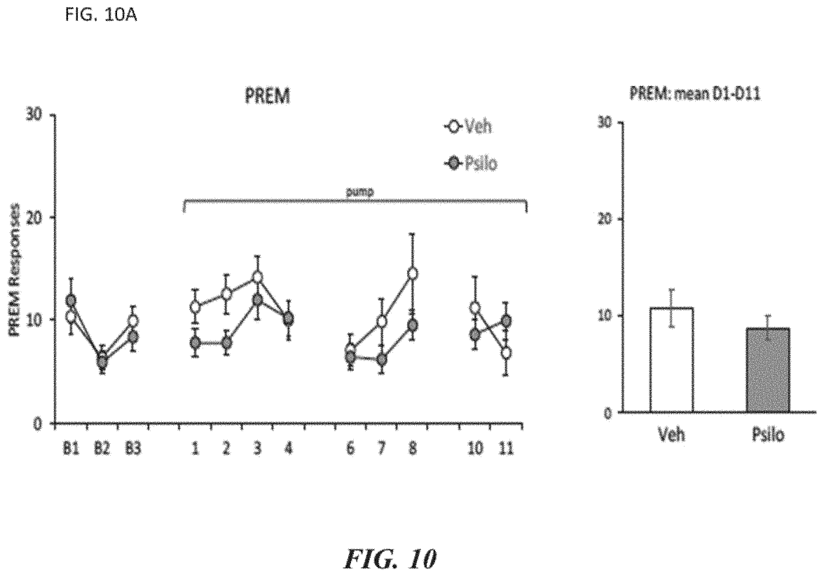

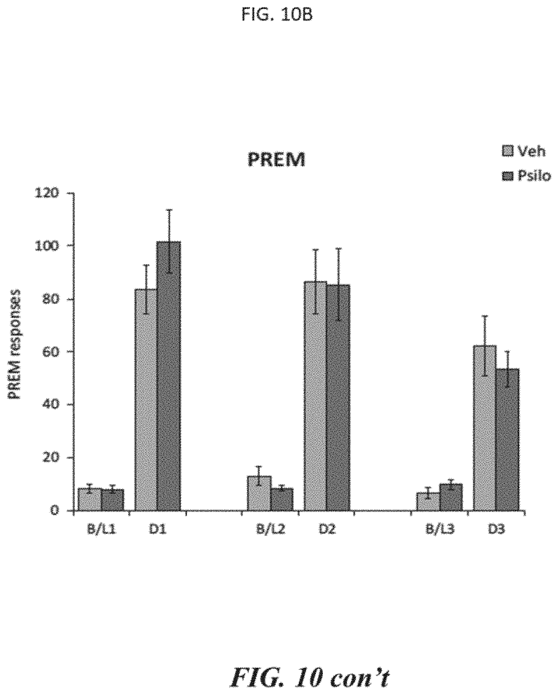

Furthermore, under the minipump (ER dosing) conditions, an increase in premature behaviors under standard conditions over the course of the study period (FIG. 10A) was not observed, nor under the more taxing conditions (10 s ITI, 0.3 s SD) (FIG. 10B). However, in the acute dosing study, a robust increase in premature attempts under the more taxing 10 s ITI, 0.3 s SD conditions was observed (FIG. 4B). So, in some embodiments, an immediate release component to any treatment that seeks to improve motivation, e.g., depression and anxiety, is preferred.

Table 18. An exemplary summary for an extended release and immediate release formulation of psilocybin or psilocin provided herein. For example, each and any one of Regimens 1, 2, or 3 can be combined (e.g., as an immediate and extended release combination) with Regimen 4, to produce a combination of immediate and extended release psilocybin or psilocin formulation.

TABLE 18

| | | | Plasma | Dose |
|---|---|---|---|---|
| Regimen | Task | Dose | [psilocin] | Regimen |
| 1 | Increase in breakpoint in food | 0.05-0.1 | ~5-12 | Acute |
| | responding under PR schedule | mg/kg | ng/mL | |
| | (low performer subgroup) | | | |
| 2 | Improvement in attention and | 0.05-0.1 | ~5-12 | Acute |
| | response speed in 5-choice test | mg/kg | ng/mL | |
| | (low performer subgroup) | | | |
| 3 | Increase in PREM/PSV | 0.05-0.1 | ~5-12 | Acute |
| | response in 10 s ITI 5-choice | mg/kg | ng/mL | |
| | test (low impulsive subgroup) | | | |
| 4 | Improvement in attention in | mini- | ~10 | Chronic |
| | 10 s ITI 5-choice test | pump | ng/mL | |
| 5 | Induction of overt 5-HT2A | ≥0.3 | ≥25 | Acute |
| | behaviors of WDS/BMC- | mg/kg | ng/mL | |
| | possible indices of | | | |
| | hallucinogenic property | | | |

PR = progressive ratio;
PREM/PSV = premature and perseverant responses, indicative of increased motivation;
WDS/BMC = wet dog shakes & back muscle contractions

### Example M. Head Twitch

A Male C57BL/6J mice are treated with compounds as below in Table 19 and Table 20 to induce Head Twitch Responses (HTRs), measured by a trained observer, for up to 20 minutes post-administration. The cumulative frequency of HTRs are recorded in real-time. The results of this study aid in the determination of an effective dose amount for each compound.

TABLE 19
Evaluation of Psilocybin, LSD and DT test compound (4 doses each)
on Head Twitch Response in the mouse (non-GLP)

| Test System | | Mouse head twitch behavior | | |
|---|---|---|---|---|
| # of Animals | | 130 male C57BL/6J mice (13 × n = 10) | | |
| Dosing Regimen | | See below | | |
| | Dose Level/ | Pre-treatment | | Number of |
| Treatment | dose volume | time | Number of mice | Animals |
| Vehicle | 10 mL/kg | 0 min | 10 | 130 mice |
| Psilocybin | 0.1 mg/kg | 0 min | 10 | total |
| Psilocybin | 0.3 mg/kg | 0 min | 10 | |
| Psilocybin | 1 mg/kg | 0 min | 10 | |
| Psilocybin | 3 mg/kg | 0 min | 10 | |
| LSD | 0.01 mg/kg | 0 min | 10 | |
| LSD | 0.03 mg/kg | 0 min | 10 | |
| LSD | 0.1 mg/kg | 0 min | 10 | |
| LSD | 0.3 mg/kg | 0 min | 10 | |
| DT test | 1 mg/kg | 0 min | 10 | |
| compound | | | | |
| DT test | 3 mg/kg | 0 min | 10 | |
| compound | | | | |
| DT test | 10 mg/kg | 0 min | 10 | |
| compound | | | | |
| DT test | 30 mg/kg | 0 min | 10 | |
| compound | | | | |

TABLE 20
Test Compound(s) and Formulation

| Test substance(s) | DMT101 |
|---|---|
| Vehicle | Saline |
| Vehicle (including preparation | Dissolve in saline |
| instructions, if required) | |
| Salt/base ratio | MW (free base) = 340; |
| | MW (salt) = 455 |
| Preparation instructions (e.g. wet grind, | No special steps |
| sonicate, stir overnight, pH etc.) | |
| Stability/storage instructions (e.g. protect | Prepare fresh solutions for |
| from light, pH, store in the fridge/freezer | immediate use |
| etc.) | |
| DT test compound | Lisuride |

### Example 6. Social Defeat

CD-1 males undergo aggression screening (mild) in order to score aggressive behavior toward C57BL/6J mice to ensure the defeat of the intruder during chronic social defeat (CSD) procedure. The C57BL/6J male mice are subjected to 10 days of CSD procedure (moderate) followed by the 1st social preference test (SP; mild) to assess CSD effect on social avoidance, the primary behavioral endpoint in the CSD model. Following baseline SP recordings, all mice undergo drug administration with either vehicle, ketamine, or psilocybin. Next, all mice groups are submitted 24 hours later to the 2nd SP test (mild) to assess acute drug effect on social avoidance. Four study groups (CTRL+vehicle, CSD+vehicle, CSD+psilocybin 0.1 mg/kg, and CSD+psilocybin 0.3 mg/kg) receive sub-chronic administration of vehicle or psilocybin for 3 more days. Again, 24 hours after last administration the 3rd SP test is performed (CTRL+vehicle, CSD+vehicle, CSD+ketamine, CSD+psilocybin 0.1 mg/kg, CSD+psilocybin 0.3 mg/kg). Table 21 illustrates an evaluation of antidepressant efficacy of psilocybin in the CSD model as set forth above. Brain tissue (mPFCx, hippocampus) is collected as presented below in Table 22. Body weight and animal welfare are monitored daily.

TABLE 21
Evaluation of antidepressant efficacy of psilocybin in CSD model

| | |
|---|---|
| Test System | chronic social defeat (CSD) induced depression-like behavior in |
| | C57BL/6J male mice |
| # of Animals | 76 male C57BL/6J (n = 10-14 per group) + 80 male CD1 mice + 6 male |
| | C57BL/6J “screener” mice |
| Dosing Regimen | Acute and sub-chronic administration of test drugs (s.c.) |

| | | | Drug administration/Dose-level | | | | # of |
|---|---|---|---|---|---|---|---|
| Group | Agent | Route | D1 | D2 | D3 | D4 | Animals |
| 1 | Vehicle | s.c. | NaCl 0.9% | NaCl 0.9% | NaCl 0.9% | NaCl 0.9% | 10 |
| 2 | Vehicle | s.c. | NaCl 0.9% | NaCl 0.9% | NaCl 0.9% | NaCl 0.9% | 10 |
| 3 | Ketamine | s.c. | Ketamine | NaCl 0.9% | NaCl 0.9% | NaCl 0.9% | 14 |
| | | | 10 mg/kg | | | | |
| 4 | Psilocybin | s.c. | Psilocybin | Psilocybin | Psilocybin | Psilocybin | 14 |
| | | | 0.1 mg/kg | 0.1 mg/kg | 0.1 mg/kg | 0.1 mg/kg | |
| 5 | Psilocybin | s.c. | Psilocybin | Psilocybin | Psilocybin | Psilocybin | 14 |
| | | | 0.3 mg/kg | 0.3 mg/kg | 0.3 mg/kg | 0.3 mg/kg | |
| 6 | Psilocybin | s.c. | Psilocybin | NaCl 0.9% | NaCl 0.9% | NaCl 0.9% | 14 |
| | | | 1.0 mg/kg | | | | |

TABLE 22

| | | First Tissue | Second Tissue |
|---|---|---|---|
| | | Collection | Collection |
| Group 1 | CTRL + Vehicle | 5 | 5 |
| Group 2 | CSD + Vehicle | 5 | 5 |
| Group 3 | CSD + Ketamine | 0 | 0 |
| Group 4 | CSD + Psilocybin dose | 0 | 14 |
| | 1 | | |
| Group 5 | CSD + Psilocybin dose | 0 | 14 |
| | 2 | | |
| Group 6 | CSD + Psilocybin dose | 14 | 0 |
| | 3 | | |

### Example 7. A Randomized, Double-Blind, Single Ascending Dose Study to Identify a Safe and Non-Psychedelic Dose of Psilocybin

A single ascending dose study is conducted that employs a randomized, double-blind, placebo-controlled design with healthy adult male and female subjects. All subjects are participating in an outpatient medical Screening Visit (Visit 1), a 3-day (2-night) inpatient Treatment Phase (Visit 2), and an outpatient Follow-Up Visit (Visit 3). For each subject, the inpatient Treatment Phase begins on the day prior to the first study drug administration (Day −1) and ends on Day 2. A medical Screening Visit will be completed within 28 days of admission to the clinical site for the Treatment Phase.

For the Treatment Phase, up to a maximum of 80 subjects are enrolled and randomized into a maximum of 10 cohorts. Ascending doses of psilocybin are evaluated in separate cohorts of 8 subjects each; within each cohort, 6 subjects each receive a single oral dose of psilocybin while 2 subjects each receive a single oral dose of matching placebo. The first cohort receives 0.5 mg of psilocybin or matching placebo; thereafter, dose levels are not fixed and are determined by the Drug Safety Review Committee upon completion of each dosing level. The dose levels include 0.5, 1, 1.5, 2, 2.5, 3, 3.5, 4, 4.5, up to a maximum dose of 5 mg. Within the planned dose range, a dose lower than the planned dose may be tested, a dose level may be repeated, or smaller dosing increments may be applied (e.g., 0.25 mg), depending on emerging safety, tolerability, and/or other relevant data, such as available pharmacodynamic data.

Within each cohort, safety and pharmacodynamic data are collected for up to 24 hours post dose. Safety monitoring includes assessments of adverse events (AEs), vital signs, electrocardiograms (ECGs), clinical laboratory tests, physical examinations, concomitant medications, and Columbia-Suicide Severity Rating Scale (C-SSRS) results. Blood samples for pharmacokinetic assessments are collected prior to and following administration of each dose of study drug. In addition, on Day −1, a sample of whole blood for potential retrospective pharmacogenetic (PGx) analysis are collected. Pharmacodynamic measures include the following assessments: the Alertness/Drowsiness visual analog scale (VAS), the Agitation/Relaxation VAS, the Hallucinations VAS, the Any Effects VAS, the Bowdle VAS, the Bond-Lader VAS, the 5 Dimensions of Altered States of Consciousness (5D-ASC) Scale, and the State-Trait Anxiety Inventory (STAI). Cognitive and psychomotor assessments are included to evaluate the impact of psilocybin on cognition (i.e., the 5-choice reaction time task [RTI], rapid visual information processing task [RVP], and spatial working memory task [SWM]). Pupil diameter are measured as an objective physiological pharmacodynamic assessment. In addition, subjects are encouraged to report any effects not being captured by the scales. These effects are documented and recorded as AEs at the discretion of the investigator.

Subjects are discharged on Day 2 following completion of all study specific procedures, on the condition that there are no medical reasons requiring a longer stay at the clinical site. In addition, subjects receive an emergency phone number to contact the clinical site for any concerns that may arise (available 24 hours per day/7 days a week).

Following completion of dosing in each cohort, available blinded safety and pharmacodynamic data are reviewed by the DSRC prior to initiating the next cohort. Proceeding to the next cohort takes place only after the safety and pharmacodynamic data is reviewed (through 24 hours postdose). The dose escalation process and any decision to stop the study are guided by specific stopping rules.

In some instances, the goal of the study is to identify a safe dose that does not elicit psychoactive effects. In some instances, the threshold dose (TD) for psilocybin is defined as the dose immediately preceding the dose at which dose escalation is stopped due to the reporting of neuropsychiatric AEs and/or the collective pattern of responses on the pharmacodynamic measures, concludes that a dose exceeding a non-psychoactive dose has been reached. In some instances, the study concludes once a TD is identified (to a maximum dose of 5 mg).

A Follow-Up Visit is performed 7±2 days after study drug administration and includes the end of study procedures.

### Criteria for Evaluation:

### Safety Endpoints:

AEs (incidence by system organ class [SOC] and preferred term [PT], severity, and relationship to study drug), serious adverse events (SAEs), and AEs leading to discontinuation; Vital signs (pulse rate, blood pressure, respiratory rate, and tympanic temperature); 12-Lead ECG; Clinical laboratory tests (clinical chemistry, hematology, and urinalysis); Physical examination findings; C-SSRS; Concomitant medications.

### Pharmacodynamic Endpoints:

The pharmacodynamic endpoints evaluated include the scores over time and derived endpoints, including maximum/minimum score (E_{max/min}), change from baseline to maximum/minimum effect (CFB_{max/min}), time to Eₘₐₓ (TEₘₐₓ), time to Eₘᵢₙ (TEₘᵢₙ), and time-averaged area under the effect curve (TA_AUE) of the following measures, where applicable: Alertness/Drowsiness VAS; Agitation/Relaxation VAS; Any Effects VAS; Hallucinations VAS; Bowdle VAS (internal and external perceptions); Bond-Lader VAS.

The 5D-ASC Scale scores are clustered into 5 dimensions as follows: oceanic boundlessness (OB), anxious ego dissolution (AED), visionary restructuralization (VR), auditory alterations (AA), and reduction of vigilance (RV). The endpoints for the 5D-ASC scale are total score at the 6-hour time point for each subscale. The STAI are clustered into state and trait anxiety subscales. The endpoints for the STAI scale are change from baseline at the 2- and 6-hour time points for each subscale. The impact of psilocybin on cognition are evaluated using 3 cognitive tasks that assess reaction time, attention, and spatial working memory, as follows: RTI, RVP, SWM.

The endpoints for the cognitive measures are scores at each time point and change from baseline to maximum or minimum effect (CFB_{max/min}; i.e., post-use-pre-use) depending on measure. Pupillometry are used as an objective physiological pharmacodynamic measure. The endpoints include maximum pupil dilation (MPD) and time to MPD (T_{MPD}).

### Pharmacokinetic Endpoints:

The following pharmacokinetic parameters are derived, where data allows: Cₘₐₓ: maximum observed plasma concentration; Tₘₐₓ: time to maximum observed plasma concentration; AUCₗₐₛₜ: area under the plasma concentration vs. time curve, from time 0 to the last measurable concentration; AUC_{INF}: area under the plasma concentration vs. time curve, extrapolated to infinity; CL/F: apparent total systemic clearance; V_{d}/F: apparent volume of distribution; T_{1/2}: terminal elimination half-life.

### Statistical Methods (Data Analysis):

The analysis populations are defined as follows: Randomized Population: All subjects who are assigned a randomization number; Safety Population: All subjects who receive 1 dose of study drug; Pharmacokinetic Population: All subjects who receive 1 dose of psilocybin and provide sufficient bioanalytical assessments to calculate reliable estimates of the pharmacokinetic parameters. Inclusion of subjects with missing data or protocol violations that may affect pharmacokinetics are considered by the pharmacokineticist on a case-by-case basis.

Safety:

At the completion of each cohort, an interim blinded listing of safety data (AEs, vital signs, and 12-lead ECG) are generated to assist the DSRC in determining dose escalation. Data are summarized by time point, as appropriate.

For the final report, descriptive statistics are used for baseline characteristics and demographic data. AEs, SAEs, and AEs leading to discontinuation are summarized by Medical Dictionary for Regulatory Activities (MedDRA) SOC and PT, severity, and relationship to study drug. All other safety data are summarized by dose level using descriptive statistics, and also listed by subject and visit (e.g., clinical laboratory data, vital signs, ECG, physical examination results, concomitant medications, and C-SSRS results). No inferential analyses are undertaken.

Pharmacodynamics:

At the completion of each cohort, an interim blinded listing of pharmacodynamic data (i.e., subjective effects VAS and cognitive measures) are generated for each subject to assist the DSRC in determining dose escalation. Data are summarized by timepoint, as appropriate. Scores overtime for select scales are shown graphically.

For the final report, pharmacodynamic data are summarized for the Safety Population by subject, dose level, and time point using descriptive statistics. Derived endpoints are summarized by dose level using descriptive statistics, and are listed by subject. No inferential analyses are undertaken.

Pharmacokinetics:

The pharmacokinetic analysis are performed using noncompartmental methods. Plasma concentrations of psilocybin and psilocin are summarized using descriptive statistics by dose level and time point, and are listed by subject. Derived pharmacokinetic parameters are listed by subject and summarized by dose level.

Dose proportionality are analyzed using a power model, with regression of the natural log-transformed Cmax, AUClast, and AUCINF in order to obtain 90% confidence intervals for the slope. Dose proportionality area concluded if the 90% confidence intervals for the slope lie entirely within 0.8 and 1.25.

Sample Size Calculation:

A formal sample size calculation was not completed. Sample size was determined based on similar studies of this type.

While preferred embodiments have been shown and described herein, it will be obvious to those skilled in the art that such embodiments are provided by way of example only. Numerous variations, changes, and substitutions will now occur to those skilled in the art without departing from this disclosure. It should be understood that various alternatives to the embodiments described herein might be employed in practicing current disclosure.

1-39. (canceled)

40. A method for improving symptoms of a cognitive or neuropsychiatric disorder, in an individual in need thereof, comprising:
a. administering to the individual a therapeutically effective amount of psilocybin or psilocin, or a pharmaceutically acceptable salt thereof, and
b. maintaining a plasma concentration of an active form of psilocybin or psilocin (i) at or above a minimum therapeutically effective threshold in the individual and (ii) below a hallucinogenic threshold in the individual for more than or equal to two hours.

41. The method of claim 40, wherein the active form of the psilocybin or psilocin is maintained below a plasma concentration in the individual of about 10 ng/mL.

42. The method of claim 40, wherein the active form of the psilocybin or psilocin is maintained above a plasma concentration of about 2 ng/mL in the individual for 2 hours or more.

43. The method of claim 40, wherein the active form of the psilocybin or psilocin is maintained above a plasma concentration of about 1 ng/mL in the individual for 2 hours or more.

44. The method of claim 40, wherein the active form of the psilocybin or psilocin is maintained above a plasma concentration of about 0.1 ng/mL in the individual for 6 hours or more.

45. The method of claim 40, wherein the active form of the psilocybin or psilocin is maintained above a plasma concentration of about 0.5 ng/mL in the individual for 6 hours or more.

46. The method of claim 40, wherein the cognitive or neuropsychiatric disorder is an anxiety, attention, or depression disorder.

47. The method of claim 46, wherein the depression disorder is major depressive disorder.

48. The method of claim 46, wherein the anxiety disorder is generalized anxiety disorder.

49. The method of claim 40, wherein the improvement of the symptoms of the cognitive or neuropsychiatric disorder ameliorates low motivation and cognitive engagement.

50. The method of claim 40, wherein the method comprises administering a therapeutically effective amount of psilocybin or psilocin, or a pharmaceutically acceptable salt thereof, at a first time.

51. The method of claim 50, wherein the method comprises administering to the individual a therapeutically effective amount of psilocybin or psilocin, or a pharmaceutically acceptable salt thereof, at a second time.

52. The method of claim 51, wherein the second time is more than or equal to 24 hours after the first time.

53. The method of claim 51, wherein the second time is more than or equal to 48 hours after the first time.

54. The method of claim 40, wherein the plasma concentration of the active form of psilocybin or psilocin is maintained (i) at or above a minimum therapeutically effective threshold in the individual and (ii) below a hallucinogenic threshold in the individual for more than or equal to 12 hours.

55. The method of claim 40, wherein the plasma concentration of the active form of psilocybin or psilocin is maintained (i) at or above a minimum therapeutically effective threshold in the individual and (ii) below a hallucinogenic threshold in the individual for more than or equal to 24 hours.

56. The method of claim 40, wherein the therapeutically effective amount of the psilocybin or psilocin, or a pharmaceutically acceptable salt thereof, is administered to the individual in need thereof as a controlled release formulation.

57. The method of claim 56, wherein the controlled release formulation comprises an extended release component that releases the active form of the psilocybin or psilocin at an amount below the hallucinogenic effective threshold and an amount of at least the therapeutically effective threshold in the individual.

58. The method of claim 57, wherein the controlled release formulation comprises an immediate release component that releases the active form of the psilocybin or psilocin at an amount below the hallucinogenic effective threshold and an amount of at least the therapeutically effective threshold in the individual.

59. The method of claim 40, wherein the therapeutically effective amount of psilocybin or psilocin, or a pharmaceutically acceptable salt thereof is administered orally.
